SEQUENCE LISTING (64 sequences)

SEQ ID NO: 1 - protein, 1051 aa, synthetic construct
MAIKETEQIPGPRPLPVVGNLFDMDLEHGLECLIRLADDFGPLFQITINGEKQIFATSQALVDELCDESRFHKAVMGGLEKLRMLASDGLFTAYHGERGWGIAHRILVPAFGPLRIRNMFEEMNDVAQQLCLKWARQGSSTSINITDDFTRLTLDTIALCTMNFRLNSFYNNETMHPFVKSMLYVLRESDIQSMLPGIANCIRVKARSRMSKHIQLMRNMARGIIQERRDQAEPVDDLLNTLLNGRDPVTGEGMSDDLIINNVITFLIAGHETTSGLLSFTFYYLLQNPHILERAQNEVDEVTGGERITVQHLGRLTYIDAILKESLRLMPTAPAFTVTPKKPEVLGGAWAIDAGQAVNVLLPVCLRDRSVFGPDADEFRPERMLEENFSKLPPNSWKPFGNGERSCIGRAFAWQEAQLVVAMVLQTFDLVPDDPSYKLRIKETLTIKPDGFRVRATLRRGQSATGLSQGSMSASGATSSVASPGPPAATGAQSNPAGGQRISFFYGSNSGTCKALAHRLASSLMGRGFTEQKLAALDTVVGNLPTDQPVIIVTTSYDGRPTDDAEEFVRWLESKRPVLQGVSYAVFGCGHHDWAKTFYRIPILIDDLMHKAGATRLTALGTANAAVSDLFSDLELWEETNLLPALREAFPPSNSSDVESSEPHQLQICVSKPRRVDMHRGLVEAKVTAVRTLTSPDSPEKRHVEFHVQGDTTWRPGDHVNILPVNPLSTVSRVLAYFQLAPDHSITVNSFNTQGLPSATPVSATELFSSFVELSQPATRKNLKALAMAAESKTDEQELIRLHDSYDALVRDKRVSVLDILERFPSISLPIGIFISMLPPLRLRTYSLSMAPSFKPSHGSLTFSVINEPAWSGNGQYLGVGSNYLASLTPGSLLYLSPRPAKDAFHLPADQFNTPIIMICAGSGLAPFMGFIQERMTWLKQGRPLAKGLLFFGCRGPHLDDLYYEELSEFEDAGVVEVHRAYSRAPDDVRAKGCRHVQHRLVTEAEAVRDHWGRNAIVYVCGSSNMARGVQTVLEEILGTLPPERYVAEIF

SEQ ID NO: 2 - protein, 1064 aa, synthetic construct
MARDAERIPGPTPLPVVGNLFDIDLEHVLQSVIGLANKYGPLFQITINGEKQIFATSQALVDELCDESRFHKAVASGLENLRMLAHDGLFTAYHGERGWGIAHRILVPAFGPLRIQSMFDDMGDLAQQLCLKWARQGASNSINITDDFTRLTLDTIALCTMDFRLNSFYNNDTMHPFVESMLYVLREADVQSALPGIANSVRIMAHRRMLKNIEAMRTIARDIIHDRRKKENPADDLLNTLLNGRDPVTGEGMSDESIIDNVITFLVAGHETTSGLLSFTFYYLVQHPDILKKAQKEVDETVGQAQISVQHLAELPYIDAILKESLRMMPTAPGFTVTPKKAETLGGKWLLNAGQPINVLLPACLRDRSIFGPNADEFSPGRMLAENFSKLPPNSWKPFGNGERSCIGRAFAWQEAQLVVAMILQNFDLVPDDPSYTLRIKETLTIKPDGFRVRATLRHRQTATGLFQHTLSARNDTSLASSSAHFIKKSEDQAPAGGRPICFFYGSNSGTCKALAHRLASDLMPYGFTDQKLAVLDTAVDNLPRDQPVIILTTTYDGQPTDDAKKFVAWLESGKVPALQGISYAVFGCGHHDWTQTFYRIPTLIDELMHKAGATRLAPRGTANAAVSDLFSDLEAWEETSLLPALRETFLLSSSSDLEPLNLHQLQISLSKPRRIDLHKDLMEARVTTVRILTNPDTPEKRHIEFRFQGDTTLRPGDHVNVLPVNPPSTVLRVLAQFNLAPDYSITINSFNTLGLPQATPVSASELFSAYVELSQPATRNNLRILAATAQSDEDKQELIHLQDSYDSLVRDKRVSVLDLLEQFPSVSLPIAAFISMLPALRLRTYSLSLAPSFKPSHGSLTFSVVNEPARNGNRRYLGVGSNYLASLTPGSILYLSPRPAKEAFHLPVDQSRIPIIMICAGSGLAPFLSFIQDRMIWQQQDKPLARALLFFGCGGRFLDDLYHEELSEFEAAGVVDVRRAYSKVLDYDMARGCKYVQDRLVAEANAIRHLWAQDATIYVCGSADMAKGVEGVLEKLLGMLPRERYVTEIYQMQTRDNVSEWLI

SEQ ID NO: 3 - protein, 1051 aa, synthetic construct
MAKDAERIPGPKPLPVVGNLFDIDPEHSLESIVAFAEKFGPLFQITINGEKQIFATSQALVDELCDELRFHKAVVTGLEILRLLAHDGLFTAYHGERGWGIAHRILVPAFGPLRIRNMLDDMSDVAQQLCLKWARQGGSTSINITEDFTRLTLDTIALCTMGFRLNSFYNNETMHPFVQSMLYVLREADIQANLPGIANSIRVSAQRRMHKNIEAMRTMARGIIQERRKNKNPVDDILNTLLNGRDPVTGEGMSDDSIIDNVITFLIAGHETTSGLLSFTFYFLIQHPHILKKAQEEVDETVGLAQISAQHLAELPYIDAILKESLRLMPTAPGFTVTPKKTEVLGGRWMINAGQPVNVLLPACLRDQSVFGPDADEFRPERMLAENFSKLPPNSWKPFGNGERGCIGRAFAWQEAQLVVAMILQTFDLVPDDPSYQLRIKETLTIKPDGFRIRATLRRGQTATGLSRRSMLVARDGSSEESSNHPAEARGDHAPARGQPVSFFYGSNSGTCKALAHQLASNMMSRGYTTQKLAPLDNAVDNLPRDQPVIILTTTYDGQPTDNAKKFVAWLETGNVLSLQGISYAVFGCGHHDWTQTFYRIPILIDDLMYKAGATRLAPRGAANAAVSDLFSDLEAWEETSLLPALRENFLPSNSTDFDPLNPHQIQLSLSKPRRVDLHKGLIEAKVTAVRVLTSPDTPEKRHLEFCFQGDLSLRPGDHLNILPVNPPSTVSRVLAQFNLAPDYNITVNSFNTLGLPQATPVSASELFSSYVELCQPATRNNLKSLIAATQSDTVKQELNRLYDSYEFIVRDKRVSVLDLLEQFPSISLPIAAFISMLPALRVRTYSLSMAPAFKPSHSSLTFSVINEPAWRGSGQHLGVASNYLASLTSGSIFYFSPRPAKETFHLPKDPSRTPIIMICAGSGLAPFLSFIQDRMVLKQQNKPLAKAFLFFGCRGRSLDDLYHEELSEYEAAGVVEVRRAYSKTPEFDIAKGCRYVQHRLVTEGQAILSLWAQNAIIYVCGSTSMAKGAEAVLQNMLGPLPKERYVTEIF

SEQ ID NO: 4 - protein, 1049 aa, synthetic construct
MAKDMESIPGPKPLPVVGNLFDIDLENGLQSIIKMAHEFGPLFQITINGQKQIFATSQALVDELCDETRFHKAVMGGIQKLRMLAKDGLFTAYHGERGWGIAHRILMPAFGPLRIRDMFEDMSDVAQQLCFKWARQGSSTSINICDDFTRLTLDTIALCTMGFRLNSYYNSNALHPFIESMLYVLKEAELQSTLPGVANCMRVKAQRRMSKHIDAMRSMARNLIEERRAKPEPVDDLLNTLLNGRDPITGEGMSDDLIISNIITFLIAGHETTSGLLSFTFYYLLQNQDVLERARNEVDEVTGVGPITVQHLAKLPYIDAIMKESLRLMPTAPAFTVTPQKPEVLGGKWMINTGDSVNLLLPVCLRDETVFGPDAGEFRPNRMLEENFSKLPPNSWKPFGNGERGCIGRAFAWQEAQLVVALVLRTFDLAAEDPYYKLRIKETLTIKPDGFRIRATLRHGKSATALSQNNISVGAAASPASSTYLAGNENGRDAAGGQPVSFFYGSNSGTCKALTHRLASTMMTRGFTDQNIAPLDSAVDNLPRDQPTIIITTTYDGQPTDDAKKFVAWLESGNSPSLQGVSYAVFGCGHQDWTKTFYRIPILIDALMYKAGATRLATRGAANAAISDLFSDLEVWEETNLLPGLRESFNPPNNSNFVPLEPHQLQISINKPTRVGMHRDLIEAKVTAIRTLTSPGAPEKRHLEFCIPGETTLRPGDHLNILPVNPPSTVSRALARFNLAPDHSITFESSNALDLPQATPVSAAELFSSYLELSQPATRNNLKELASTTPSDGEKQELLHLYDSYDSLIRAKRASVLDLLEQFTSVTLPITTFISMLPALRVRTYSLSMAPSFKPLHYSLTFSVINEPAWNGNGRYLGVASNYLASLNLGSILYISPRPAKDAFHLPTDQSSKPIIMICAGSGLAPFRSFIQDRMLWQQQDKTLAKALLFFGCRSPQLDDLYHDELSQFEAAGVVEVRRAYSKVPNHYLAKGCRYVQHRLLTETETIQDMWAQDAIIYVCGSGNLAKGVKAVLESMLGTLSERYITEIF

SEQ ID NO: 5 - protein, 1051 aa, synthetic construct
MAKDAERIPGPKPLPVVGNLLDIDPEHGLQSIIAFADKYGPLFQITINGEKQIFATSQALVDELCDESRFHKAVVTGLEVLRLLAHDGLFTAYHGERGWGIAHRILVPAFGPLRIRNMLDDMSDVAQQLCLKWARQGGSTSINITEDFTRLTLDTIALCTMGFRLNSFYNNETMHPFVQSMLYVLKEADVQANLPGIANSIRVSAQRRMHKNIEAMRTMARGIIQERRKNKNPVDDILNTLLNGRDPVTGEGMSDDSIIDNVITFLIAGHETTSGLLSFTFYFLIQHPHILKKAQEEVDETVGLAQISAQHLAELPYIDAILKESLRLMPTAPGFAVTPKKTEVLGGKWMINAGQPVNVLLPACLRDQSVFGPDADEFHPERMLAENFSKLPPNSWKPFGNGERGCIGRAFAWQEAQLVVAMILQTFDLVPDDPSYQLRIKETLTIKPDGFRIRALLRRGQTATGLSRRSMLVARDGSSGESSNHLAEARGDHAPARGQPVSFFYGSNSGTCKALAHQLASNMMSRGYTTQKLAPLDNAVGNLPRDQPVIILTTTYDGQPTDDAKKFVAWLETGNVPSLQGISYAVFGCGHHDWTQTFYRIPILIDDLMHKAGATRLAPRGAANAAVSDLFSDLEAWEETSLLPALRENFLPSNSTDFDPLNPHQIQLSLSKPRRVDLHKGLIEAKVTAVRVLTSPDTPEKRHLEFCFQGDTSLRPGDHLNILPVNPPSTVSRVLAQFNLAPDYNITVNSFNTLGLPQATPVSASELFSSYVELCQPATRNNLKALIAATQSDPDKQELNRLYDSYEFIVRDKRVSVLDLLEQFPSISLPIAAFISMLPALRVRTYSLSMAPSFKPSHSSLTFSVINEPAWRGSGQHLGVASNYLASLTSGSIFYFSPRPAKESFHLPKDPSNTPIIMICAGSGLAPFLSFIQDRMVLKQQYKPLAKAFLFFGCRGRSLDDLYHEELSEFEAAGVVEIRRAYSKTPDFDIAKGCRYVQHRLVTEGQAILSLWSQNATIYVCGSTNMAKGVEAVLQNMLGPLPKERYVTEIF

SEQ ID NO: 6 - protein, 1050 aa, synthetic construct
MAKDMDCIPGPKPLPVVGNLFDLDLDNALQSIIRMADEFGPLFQITINGQKQIFATSQALVDELCDETRFHKAVMGGVEKLRMLAQDGLFTAHHGERGWGIAHRILMPAFGPLRIRDMFEDMSDVAHQLCFKWARQGSSASINIAEDFTRLTLDTIALCTMSFRLNSYYNSETMHPFVQSMLYVLKEADLQATLPGVANCVRVKAQRRMSKHIQAMRNIAGDIIKGRRDKPEPVDDLLNTLLNGRDPVTGEGMSDELIISNIITFLVAGHETTSGLLSFTFYYLLQHPHVLEQARNEVDEVVGVGPITVQHLAKLPYIDAVMKESLRLMPTAPAFTVTPKKPEVVGGKWMVNTGQSVHVLLPVCLRDEAVFGPDAGEFRPTRMLEENFSKLPPNSWKPFGNGERGCIGRAFAWQEAQLVVASVLQTFDLVAEDPYYKLRIKETLTIKPDGFRVRATLRRGQSATALSQHNMSAGATASPGSSTHLAGDENGQDTAGGQPISFFYGSNSGTCKALAHRLASTMMTRGFTDQHLAQLDSAVDNLPRDQPTIIVTTTYDGQPTDDAKKFLAWLESGNVPSLHGVSYAVFGCGHQDWTKTFYRIPILIDDLMHKAGATRLTTRGTANAAVSDLFSDLEVWEETNLLPALREKFYLCNSSDFEPLDPHQLQISISKPARVGMHRDLVEGKVTAIRTLTSPGVPEKRHVEFQIPSEMALRPGDHVNILPVNPPCSVLRALARFSLASDHSITFESSNALDLPQATPVSAAELFSSYLELSQPATRINLKSLASATPSDDDKKELLHFHDSYDSLIRDKRVSVLDLLEHFTSITLPIATFISMLPVLRVRTYSLSMAPSFKPLHCSLTFSVVNEPAWSGNGRYLGVGSNYLASLTPGSILYVSPRPAKDAFHLPTDQSSNPIIMICAGSGLAPFRSFIQDRMAWLQQGKPLAKALLFFGCRGPHLDDLYHDELSEFESAGVVEVRRAYSKVPNHYLAKGCRYAQHRLLTETETIQDMWAHNATLYLCGSANLAKGVKAVLENMLGTLSEERYITEIF

SEQ ID NO: 7 - protein, 1050 aa, synthetic construct
MAKDMDCIPGPKPLPVVGNLFDLDLDNALQSIIKMADEFGPLFQITVNRQKHIFATSQALVDELCDETRFHKAVIGGVEKLRMLAHDGLFTAHHGERGWGIAHRILMPAFGPLRIRDMFEDMSDVAHQLCFKWARQGSSTSINISEDFTRLTLDTIALCTMSFRLNSYYNSDTMHPFVQSMLYVLKEADLQSSLPEVANCVRVKAQRSMSKHIEAMRSIAGDIIKGRRDKPEPVNDLLNTLLNGRDPVTGEGMSDELIISNIITFLIAGHETTSGLLSFTFYYLLQHPQVLEQARNEVDEVVGVGPITVQHLAKLPYIDAIMKESLRLMPTAPSFTVTPKKPEVLGGKWMINPGQSVHVLLPVCLRDEAVFGPDAGEFRPNRMLEENFSKLPPNSWKPFGNGERGCIGRAFAWQEAQLVVASVLQTFDLVAEDPNYKLRVKETLTIKPDGFRVRATLRHGRSATALSQHNMSAGATSSPGSSAHPAGNKNAQDAAGGQSISFFYGSNSGTCKALAHRLASTMMTRGFTDQHLAPLDSAVDNLPKDQPTIIVTTTYEGQPTDDAKKFLAWLESGIVPSLHGVSYAVFGCGHQDWTKTFYRIPILIDDLMHKAGATRLTTRGEANAAVSDLFSDLEVWEETNLLPALREKFDASNSGEFESLDLQQLQISISKPTRVGMHRDLIEGKVTAIRTLTSPGVPEKRHVEFQITSDTTLRPGDHVNILPVNPPSTVLRALARFNLASDHIITFESSNALDLPQATPVSAAELFGSYLELSQPATRNNLKSLASTTPSDEDKQELLRFHDSYDSLIRDKRVSVLDLLEHFTSITLPIATFISMLPVLRVRTYSLSMAPSFKPLHCSLTFSVVNEPAWSGNGRYLGVGSNYLASLTPGSILYVSPRPAKEAFHLPADQSSKPIIMICAGSGLAPFRSFIQDRMAWLQQGKPLAKALLFFGCRGPQLDDLYHDELSEFESAGVVEVRRAYSKVPNHYPGKGCRYVQHRLFAETETIQDMWAHNATLYLCGSATLAKGVKATLENMLGTLSEERYITEIF

SEQ ID NO: 8 - protein, 1056 aa, synthetic construct
MARDAERIPGPKPLPVVGNLFDIDLECVLQSVIELANEYGPLFQITINGEKQIFATSRALVDELCDESRFHKAVVSGLENLRMLAHDGLFTAYHGERGWGIAHRILVPAFGPLRIQNMFDDMGDVAQQLCLKWARQGSSTSINITDDFTRLTLDTIALCTMDFRLNSFYNNETMHPFVQSMLYVLREADLQSSLPGIANSVRVKAQRRMSKNIEAMRSLARDIIHDRRKKANPADDILNTLLNGRDPVTGEGMSDDSIIDNVITFLVAGHETTSGLLSFTFYYLVQHPHILQKAQKEVDETVGRAQISVQHLAELPYIDAILKESLRLMPTAPGFTVSPKKTEVLGGKWMINAGQPINVLLPACLRDQSVFGPNADEFCPERMLAENFSKLPPNSWKPFGNGERSCIGRAFAWQEAQLVVAMILQNFDLVPDDPSYKLRIKETLTIKPDGFRVRARLRHGQTATGLFQRTMSARADARLGSSSPAYLPNKSEGHAPAEGRPISFFYGSNSGTCKALAHRLASNMMSYGFTDQKLAPLDTAVDNMPRDQPVIILTTTYDGQPTDDAKKFVAWLESGKVPALQGISYAVFGCGHHDWTQTFYRIPNLIDELMHKAGATRLAPRGTANAAVSDLFSDLEAWEETSLLPGLREASLLSSSSSSCRDSPNPHQLQISLSKPRRIDLHKGLIEARVTAVRILTSPDTPEKRHVEFCFQGDATLRPGDHLNVLPVNPPSTVLRVLAQFNLAPDYSITVNSFNTLGLPQATPVSASELFSSYVELSQPATRNNLRILAATAQSDADKQELVHLQDSYDSLVRDKRVSVLDLLEQFPSISLPIAAYISMLPALRVRTYSLSMAPSFKPSHGSLTFSVINEPARNGNGQYLGVGSNYLASLTPGSILYLSPRPAKEPFHLPRDQSRTPIIMICAGSGIAPFLSFIQDRMVWQQQQEKPPLAKALLFFGCRGRSLDDLYHDELSEFEAAGVVEVRRAYSRIPHHDIARGCKYVQHRLVAEAKAIRYLWAQDATIYVCGSGEMARGVETVLENMLGGLPRERYVTEIF

SEQ ID NO: 9 - protein, 1051 aa, synthetic construct
MAIKETEYIPGPRPLPVVGNLFDMDLEHGLESLIGLADEFGPLFQITINGEKQIFATSQALVDELCDESRFHKAVIAGLEKLRMLANDGLFTAYHGERGWGIAHRILMPAFGPLRIRSMFEEMSDVAQQLCLKWARHGSSTSINITDDFTRLTLDTIAPCTMDFRLNSFYNTETMHPFVKSMLYVLKESDIQSMLPGIANRIRVKARGRMSKHIQLMRDMARGIIQERRAQAEPVDDLLNTLLNGRDPVTGEGMSDDLIINNVITFLIAGHETTSGLLSFTFYYLLQTPRILERAQNEVDEVIGSERITVQHLGRLPYIDAILKESLRLMPTAPAFTVTPKKPEVLGGTWAIDVGQAVSVLLPACLRDRSVFGPDADEFRPERMLEENFSKLPPNSWKPFGNGERSCIGRAFAWQEAKLVVAMLLQTFTLVPDDPSYKLRIKETLTIKPEGFRVRATLRRGQSATGLSQCSMLATGATGAVASPGRPAAPGAHPNPGGGQRISFLYGSNSGTCKALAHRLASSLMGRGFTEQTLAALDTVVGNLPTNQPVIIVTTSYDGRPTDDAEEFFRWLESKRPVLQGVFYAVFGCGHHDWAKTFHRIPILIDELMHSAGATRLTALGTANAAVSDLFSDLELWEETNLLPALREAFPPSSSSDVESSDPPQVHISVSKPRRVDMHRGLVEAKVTAVRTLTSPDSPEKRHVEFHIQGDTTWRPGDHVNILPVNPPSTVSRVLDYFQLAPDHSITVNSFNTQGLPSATPVSAVELFSSFVELSQPATRKNLKALAMAAESKTEEQELIRLHDSYDALVRDKRVSVLDILERFPSISLPISTYISMLPPLRLRTYSLSAAPSFKPSHGSLTFSVINEPAWSGNGQYLGVGSNYLASLTPGSLLYLSPRPAKDAFHLPADLSTTPIIMICAGSGLAPFMGFIQERMTWLKQGRPLAKALLFFGCRGPHIDDLYYEQLSEFEDAGVVEVHRAYSRAPDDVQAKGCRYVQHRVVTEAVAVRDHWGQNAIVYVCGSSNMARGVRTMLEGILGTLPQERYIAEIF

SEQ ID NO: 10 - protein, 1050 aa, synthetic construct
MAKDMECIPGPKPLPVVGNLFDIDLEHGLQSIVEMADEFGPLFQITINGKKQIFATSQALVDELCDETRFHKAVIAGIQKLRMLANDGLFTAYHGERGWGIAHRILMPAFGPLRIRDMFDDMSDVAQQLCFKWARQGSSTSINISDDFTRLTLETIALCTMGFRLNSFYNSESMHPFVKSMLYVLKEADLQSTLPGIANSMRVKAQRHMSKNIEAMRSMAGDIIKERRDKPESVDDLLNTLLNGRDPVTGEGMSDDLIISNIITFLIAGHETTSGLLSFTFYYLLQHPLVLEAAQNEVDEIAGVDPITVQHLAKLPYIDAIMKESLRLMPTAPGFTVTPKKPEVLGGKWRINPGQSVNLLLPVCLRDQTVFGPDAGEFRPERMLEENFSKLPPNSWKPFGNGERGCIGRVFAWQEAQLVVAMVLQTFDLVADDPSYELRIKETLTIKPDGFRVRAMLRRGQSATGLSQRRMSAGAAASLASSTHLTGNGNGQEAAGGQPVSFFYGSNSGTCKALTHRLASTMMARSFTDQKLAPLDSAVNNLPRDQPVIIITTTYDGQPTDDAKKFVAWLESGNVPSLQGVFYAVFGCGHQDWTKTFYRIPILIDNLMHNAGATRLATRGAANAAVGDLFSDLEAWEETSLLPALRETFLLSSSSDIEPLDLHQLQISLCKPTRVEMHQGLVEAKVTAIRKLSSTGTPEKRHIEFHILGEATLRPGDHLNVLPVNPPSAVSRVLARFHLAPDHGITIKSFNTLGLPHATPVSAAELFSSYLELSQPATRNNLKALALSAPSDGDKRDLLRFYDSYDSLIRDKRVSVLDLLEQFPTVPLPIAAFISMLPALRVRTYSLSTAPSFKPSHGSFTFSVVNEPAWNGNGRYLGVGSNYLASLTLGSILYISPRPAKEAFHLPTDQYSTPIIMACAGSGLAPFRSFIQERMFWLQQGKPLAKALLFFGCRGPHLDDLYDEELSEFESAGVVKVRRAYSKAPDHYLAKGCRYVQHRLAAETEAIQDLWARNATIYVCGSGDLAKGVKAVFENMLGTLSEERYVTEIF

SEQ ID NO: 11 - protein, 1051 aa, synthetic construct
MAKDTEQIPGPKPLPVVGNLFDIDLEHGLESIIEIANNFGPLFQITINGEKQIFATSQALVDELCDETRFHKAVVAGVQNLRMLANDGLFTAYHGERGWGIAHRILMPAFGPLRIRNMFEDMSDIAQQLCYKWARQGSSISINITDDFTRLTLDTIALCTMGFRLNSFYNNETMHPFVQFMLHVLREADVQAMLPGIIKSIRIKAQQRMSKNIEAMRSIARQIIQERRDKGEPADDLLNTLLNGRDPVTGEGMSDDSIIDNVITFLIAGHETTSGLLSFTFYYLLQNPHILEQARNEVDDVVGVDQITVQHLAKLPYIDAILKETLRLMPTAPAFAVTPKKPEVLGGKWTINTGQPVNVLLPVCLRDQAVFGPDAGEFRPERMLEENFSKLPPNSWKPFGNGERSCIGRAFAWQEAQLVVAMVLQTFDLVSDDPSYKLRIKETLTIKPDGFRVRATLRRGQSATGLSRRSMLVSADGSSAASSTHLAGNCKGQEPAGGQPVSFFYGSNSGTCKALAHRLASNMMARGFTSKRLAPLDSAVDTLPKDQPVIILTTTYDGQPTDDAKKFVAWLESGKVPALQGVSYAVFGCGHQDWTKTFYRVPILIDDLMYKAGATRLAARGAANAAISDLFSDLEEWEETSLLPALRETFLPSSSSGVELLNPHQLQISLSKPRRVGMHQGFVEAKVTAVRILTSPGTPQKRHVEFRIQGETTLRPGDHLNILPVNPPGTVSRVLAHFDLAPDYSITINSFNTLGVPHATPVLAAELFSSYVELSQPATRNNLKVLTATAQSDADKQELICFHDSYNSLVRDKRVSVLDLLEQFPSISLPIAAFISMLPALRVRTYSLSMAPSFKPSHSSLTFSIVNEPAWSGNGQYLGVGSNYLASLTLGSILYISPRPAKDDFHLPIDQSSTPIIMICAGSGLAPFLSFIQDRMVRLQQGKPLAKALLFFGCRGPHLDDLYHEELSEFESAGVVEVHRAYSKAPDHDLAKGCRYVQHRLVTEAEAIQDLWARNAKVYVCGSGNLAKGVQTALKNMLGALPQERYVTEIF

SEQ ID NO: 12 - protein, 780 aa, synthetic construct
MAETELKETARGTCPVAHGGQSSVGGCPVHRLAEDFDPFQDAYMADPAQFVRWAREQVPIFYAPKLNYWVVTRYDTIKQIFRDPVTFSPSNVLQSFAQPSAEVRQVLERYGYAFNRTLVNEDEPMHLERRRVLMEPFASEHLAEHEPMVRELVRRAVNRFIDTGRADLVDQMIWEVPFTVALHFLGVDDDDREKMRRFAIAHTVNAFGRPSPEEQLAVAETVGQFWQFCGEVLEKMRRTADGPGWMRYSIRQQKLYPDVVTDSYLHSMMQAIIVAAHETTVFATTNALKTLLEHETVWREICADPSLIPAAAEECLRYNGPVAGWRRRTTREVEVEGVRLPVGANILMVVASANHDSAHFDDPEFFDIGRSNASEHLNFGYGAHQCLGRNLGRMEMQIMIEELSRRLPHMRLAEQRFDYLHNVSFRAPRHLWVEWDPAQNPERRDPDILRLRQPVRIGPPRAKDVVRTMEVAAVERPSEDIVVLHLTRPDRRPLPRWSPGAHIDIECGEPDRSRQYSLCSDPENRDAWRVAVQRDPASRGGSRWIHEEVRPGMLLRVRGPRNSFRLDEHAPRYLFLAGGIGITPIMTMAARAKELGTDYELHYSVRSRTSLIFVDELRQIHGDRLHVYVSEEGVRNDLAALIRRASAGTQIYACGPQRMLDTLERLIENRPEVTLRVEHFFGEPSHLDPAKERPFQVVLRNSGLTVEVPADKTLLEVLRAYNIEVQSDCEEGLCGTCEVSVVEGEVDHRDSVLTRAERRENRRMMCCCSRAKTERLVLDL

SEQ ID NO: 13 - protein, 767 aa, synthetic construct
MAHGDETPLRSPTGCPISPRAAAFRPFERPYQEDPAAALAWARAEEPVFYAPDIGYWVVTRHEDVKAVFRDNITFSPRIALEKITPPTEEVQAILKRYGYNLQRTMVNEDEPDHMERRRLLMADFLPERLARHEPMVRRLTRAHMDRFIDRGHADLVAEIFRDVPLKVALHFLGVAEEDADELTRFAAAHTLNTWGRPTPEEQREIAENVGRFWQKANAILDRMIADPSGEGWMYETVRQHFRHPDIVPESYMRSMMMAILVAAHETTTLATANAFRLLLERRDAWEELCANPGLIPSAVEECLRMGGSIIAWRRIATREAEVGGVTIPEGGRLLLVMASANRDESHWENPEELDIFRDNAVEHLSFGYGAHQCMGKNIARMEMRVFLDEFTRRLPHMRLKPGQSFEGLCNIAFRGPKSLLVEWDPAENPERREPALRERAVSFPIGAPVKENILRRVVIVEAREEAEGVKSFLLADPRGRPLPGWSAGAHVDLVSGGFRRKYSLCSAPGEEGRWRIAILREAEGRGGSRHFCDALSEGAEVQVAGPRNHFRLDESAARYRLIAGGIGITPILAMADRLKVLGRDYTLHYCGPSRARMAFLDRVLADHGPRAQLHVSDEGTRLDLAGEFAAVTEGEQVYACGPARMLDALRELARGWPEGALHFEYFTTGAATLDPAREHAFEAVLADSGLVVEVAADETLYAALRRAGIDIQSDCGEGLCGSCEARVVEGEIDHRDRVLSRAERARGDRMMTCCSRAKGRRIVLGL

SEQ ID NO: 14 - protein, 770 aa, synthetic construct
MATSSTAPCPHGVAPNGCPISADAAAFRPFDGPYQLDPAEALRRAREREPVFYAPDIGYWVVTRYEDVKAVFRDNNLFSPAIALEKMTPPTAEAQAILRKYNYNLQRTMVNEDEPQHMERRRLLLDDFLPERLARHEPMVRRLCRDYMDRFIDRGHADLVNDIFYEIPLTVALHFLGVPDEGAERLRQFAVAHTLNTWGRPPPEEQIEIAENVGRFWQTANEILDDMIANPDGEGWMYETVRQHFKHPDIVPESYMRSMMMAILAAAHETTSNATANAFWTLLTHRDAWEEICENPALIPSAVEECLRVAGSIIAWRRIATDDAELGGVKIPKGGKLLIVQASANRDAAHWENPDQVDIYRDNAVEHLTFGYGAHQCMGKNIGRMEMRVFLEEFTRRLPHMRLVPGQTFENLRNISFRGPARLLVEWDPAQNPERHDPSILDRAMSFPIGAPVRDDTLRRVVVQEVRDEATGVRSYRLADPRGKALPKWTPGSHVDLVAGGFRRKYSLCGDAQDSSTYQIAILREEDGRGGSRHFHDTLEAGATVHIAGPRNHFRLDEGAESYVLVAGGIGITPILAMADRLRTLGRPYRLHYCGATRAAMALLERVMRDHGDRLRLHVSDEGTRLDMAAELTGVGAGTQVYACGPERMLEALQDLARGWPEGTLHYEYFSAGASGLDPSREHGFEVVLADSDMTIQVAPDETVFEALRRAGVDLQTDCGEGLCGTCEARVLEGEIDHRDRVLSRAERDRGDRMMTCCSRARGRRLVLAL

SEQ ID NO: 15 - protein, 511 aa, synthetic construct
MAILVLASLFAVLILNVLLWRWLKASACKAQRLPPGPPRLPILGNLLQLGPLPHRDLASLCDKYGPLVYLRLGNVDAITTNDPDTIREILLRQDDVFSSRPKTLAAVHLAYGCGDVALAPMGPHWKRMRRICMEHLLTTKRLESFTTQRAEEARYLIRDVFKRSETGKPINLKEVLGAFSMNNVTRMLLGKQFFGPGSLVSPKEAQEFLHITHKLFWLLGVIYLGDYLPFWRWVDPSGCEKEMRDVEKRVDEFHTKIIDEHRRAKLEDEDKNGDMDFVDVLLSLPGENGKAHMEDVEIKALIQDMIAAATDTSAVTNEWAMAEAIKQPRVMRKIQEELDNVVGSNRMVDESDLVHLNYLRCVVRETFRMHPAGPFLIPHESVRATTINGYYIPAKTRVFINTHGLGRNTKIWDDVEDFRPERHWPVEGSGRVEISHGPDFKILPFSAGKRKCPGAPLGVTMVLMALARLFHCFEWSSPGNIDTVEVYGMTMPKAKPLRAIAKPRLAAHLYT

SEQ ID NO: 16 - protein, 1050 aa, synthetic construct
MATIKEMPQPKTFGELKNLPLLNTDKPVQALMKIADELGEIFKFEAPGRVTRYLSSQRLIKEACDESRFDKNLYQALKFARDFAGDGLATSWTHEKNWKKAHNILLPSFSQQAMKGYHAMMVDIAVQLVQKWERLNADEHIEVPEDMTRLTLDTIGLCGFNYRFNSFYRDQPHPFITSMVRALDEAMNKLQRANPDDPAYDENKRQFQEDIKVMNDLVDKIIADRKASGEQSDDLLTHMLNGKDPETGEPLDDENIRYQIITFLIAGHETTSGLLSFALYFLVKNPHVLQKAAEEAARVLVDPVPSYKQVKQLKYVGMVLNEALRLWPTAPAFSLYAKEDTVLGGEYPLEKGDELMVLIPQLHRDKTIWGDDVEEFRPERFENPSAIPQHAFKPFGNGQRACIGQQFALHEATLVLGMMLKHFDFEDHTNYELDIKETLTLKPEGFVVKAKSKKIPLGGIPSPSTEQSAKKVRKKAENAHNTPLLVLYGSNMGTAEGTARDLADIAMSKGFAPQVATLDSHAGNLPREGAVLIVTASYNGHPPDNAKQFVDWLDQASADEVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSTNVVASKELQQPGSARSTRHLEIELPKEASYQEGDHLGVIPRNYEGIVNRVTARFGLDASQQIRLEAEEEKLAHLPLAKTVSVEELLQYVELQDPVTRTQLRAMAAKTVCPPHKVELEALLEKQAYKEQVLAKRLTMLELLEKYPACEMKFSEFIALLPSIRPRYYSISSSPRVDEKQASITVSVVSGEAWSGYGEYKGIASNYLAELQEGDTITCFISTPQSEFTLPKDPETPLIMVGPGTGVAPFRGFVQARKQLKEQGQSLGEAHLYFGCRSPHEDYLYQEELENAQSEGIITLHTAFSRMPNQPKTYVQHVMEQDGKKLIELLDQGAHFYICGDGSQMAPAVEATLMKSYADVHQVSEADARLWLQQLEEKGRYAKDVWAG

SEQ ID NO: 17 - protein, 1076 aa, synthetic construct
MAIKETEQIPGPRPLPVVGNLFDMDLEHGLECLIRLADDFGPLFQITINGEKQIFATSQALVDELCDESRFHKAVMGGLEKLRMLASDGLFTAYHGERGWGIAHRILVPAFGPLRIRNMFEEMNDVAQQLCLKWARQGSSTSINITDDFTRLTLDTIALCTMNFRLNSFYNNETMHPFVKSMLYVLRESDIQSMLPGIANCIRVKARSRMSKHIQLMRNMARGIIQERRDQAEPVDDLLNTLLNGRDPVTGEGMSDDLIINNVITFLIAGHETTSGLLSFTFYYLLQNPHILERAQNEVDEVTGGERITVQHLGRLTYIDAILKESLRLMPTAPAFTVTPKKPEVLGGAWAIDAGQAVNVLLPVCLRDRSVFGPDADEFRPERMLEENFSKLPPNSWKPFGNGERSCIGRAFAWQEAQLVVAMVLQTFDLVPDDPSYKLRIKETLTIKPDGFRVRATLRRGGNATDFQSELQSLGTQAKGVRSDSAVSMSSAQADKGDKKPLTILYGSNSGTCEALAYRLASDAALHGFYARKIAPMNAACNDLPKSEPVIILAASYDGSPSDNAVEFFEWLNKAEPESLKGVSYTVFGCGHRDWVATFQRIPILIDDLLEKAGADRFADRGLADAAVMDLFVELEKWTTDVVWPAISQNEGGRDDDGETRLINSLLKLEVSQPRQLRQYSQLVQATVTESRSLTTEAALGKKMHLDMKLPPGVSYHPGDHLLVLPVNPPRDVKRVLSRFRLAWDTVVRASGEQYAHIPVETSMTAHELLSSYFELSQPATPRDIRVLAAAATDQKTKHLLSNLATASYAEGIHENRVSLLDLLEEFADIPLAFGTFIALLPSLRLRTYSISSSPSWEPNHASLTFSILDEPPTPPRTKSFLGVASNYLANLTPGDLIHVATRPVKNIFQMPADVSKVPIIMVAAGAGVAPFRGFIQERASQQQNGTELAPAALFFGCRSSDDDLYHHELDYFEDSGVVQVFRAYSREEDAGSQPSTRKGYVQESLLAEKQAFLRLWNAGAKIYVCGGVRMASGVKEAVMKLVYMAEQDATSKSFTPQEWFKRFEKARYAAEIFAA

SEQ ID NO: 18 - protein, 1073 aa, synthetic construct
MAIKETEQIPGPRPLPVVGNLFDMDLEHGLECLIRLADDFGPLFQITINGEKQIFATSQALVDELCDESRFHKAVMGGLEKLRMLASDGLFTAYHGERGWGIAHRILVPAFGPLRIRNMFEEMNDVAQQLCLKWARQGSSTSINITDDFTRLTLDTIALCTMNFRLNSFYNNETMHPFVKSMLYVLRESDIQSMLPGIANCIRVKARSRMSKHIQLMRNMARGIIQERRDQAEPVDDLLNTLLNGRDPVTGEGMSDDLIINNVITFLIAGHETTSGLLSFTFYYLLQNPHILERAQNEVDEVTGGERITVQHLGRLTYIDAILKESLRLMPTAPAFTVTPKKPEVLGGAWAIDAGQAVNVLLPVCLRDRSVFGPDADEFRPERMLEENFSKLPPNSWKPFGNGERSCIGRAFAWQEAQLVVAMVLQTFDLVPDDPSYKLRIKETLTIKPDGFRVRATLRRGGNATDLQSELQSSGTKTKGVRTDSAVSITSAQADKKNKKPLTILFGSNSGTCEALAYRLASDAALHGFYARRIAPMNAACNNLPKSEPVIILAASYDGSPSDNAVEFFEWLNKVEPDSLKGVSYTVFGCGHRDWVATFQRIPILIDDLLEKAGADRFADRGLADAAVMDLFVELEKWTTDVVWPAISQTEGGGDGGESRLINSLLKVEVSQPRQLRQYSQLVQATVTETRSLTTESAIGKKMHLGIQLPPGVSYHPGDHLLVLPVNPPRDVKRVLSRFHLAWDTVVRASGDESVHIPVETSMTAHELLSSYFELSQPATPRDIRVLAAAATEQKTRHLLSSLATASYAEGIHENRVSLLDLLEEFPDIPLAFGTFIALLPSLRLRTYSISSSPSWEPNHASLTFSVLDEPPTPPRSKSFLGVASNYLANSTPGDLIHVATRPVKSIFQMPTDASKVPVIMVAAGAGLAPFMGFVQERASQQRNGTELAPAALFFGCRSSHDDLYHHELDCFEDSGAVQVFRAYSREDAGSKPNTRKGYIQDALLAEKQAFLRLWNAGAKIYVCGGVRMASGVKEAVMKLVYMAEQDAASKSFTPQEWFKRFEKTRYAAEIFT

SEQ ID NO: 19 - protein, 1077 aa, synthetic construct
MAIKETEQIPGPRPLPVVGNLFDMDLEHGLECLIRLADDFGPLFQITINGEKQIFATSQALVDELCDESRFHKAVMGGLEKLRMLASDGLFTAYHGERGWGIAHRILVPAFGPLRIRNMFEEMNDVAQQLCLKWARQGSSTSINITDDFTRLTLDTIALCTMNFRLNSFYNNETMHPFVKSMLYVLRESDIQSMLPGIANCIRVKARSRMSKHIQLMRNMARGIIQERRDQAEPVDDLLNTLLNGRDPVTGEGMSDDLIINNVITFLIAGHETTSGLLSFTFYYLLQNPHILERAQNEVDEVTGGERITVQHLGRLTYIDAILKESLRLMPTAPAFTVTPKKPEVLGGAWAIDAGQAVNVLLPVCLRDRSVFGPDADEFRPERMLEENFSKLPPNSWKPFGNGERSCIGRAFAWQEAQLVVAMVLQTFDLVPDDPSYKLRIKETLTIKPDGFRVRATLRRGGTATDFQRELHSVGSTHDVRPSLTPSTNTLSQAGEGNKKPLTIFFGSNSGTCEALAYRLASDATLHGYYARTIAPLNAACGNLPKSEPVLILAASYDGLPSDNAEEFFDWLNKAEKDSLNGVSYAVFGCGHRDWVATFQRIPILIDDLLQRAGAERFAARGLADSAVMDLFVELEKWTADSVWPAITQTDGGGGERDDDGEGRLITSLLKVDFSQPRQLKQYSQLVEASVTESRTLTTASAIGKKVHLDIQLPSDQSYSPGDHLLVLPVNPMQTVKRVLSRFHLTWDTIVRASGNDSVRIPADTSMTAYELLSSYFELSQPATPRDIRVLAAAAEDEATKQLLTDLATSSYVEGIQEKRTSLLDLLERFPEIPMALGTYLTLLPSLRLRTYSISSSPSWNPGHATLTLSVLEEPATADCGSHFLGVASNHLAGLSRGDLIHVATRPVKGIFRMPTDLSKVPIIMIAAGSGLAPFRGFIQERAIQQRNGTELAPAALFFGCRSSDDDLYRSELDEFESAGVVRVFRAYSREDVGSRPNTRKGYVQDSMTAEKDAFAQLWNAGAKIYVCGSVRMASQVKDVVTKLVYAAETLGASKQFTPQEWFKRFEKTRYAAEIFT

SEQ ID NO: 20 - protein, 1071 aa, synthetic construct
MAIKETEQIPGPRPLPVVGNLFDMDLEHGLECLIRLADDFGPLFQITINGEKQIFATSQALVDELCDESRFHKAVMGGLEKLRMLASDGLFTAYHGERGWGIAHRILVPAFGPLRIRNMFEEMNDVAQQLCLKWARQGSSTSINITDDFTRLTLDTIALCTMNFRLNSFYNNETMHPFVKSMLYVLRESDIQSMLPGIANCIRVKARSRMSKHIQLMRNMARGIIQERRDQAEPVDDLLNTLLNGRDPVTGEGMSDDLIINNVITFLIAGHETTSGLLSFTFYYLLQNPHILERAQNEVDEVTGGERITVQHLGRLTYIDAILKESLRLMPTAPAFTVTPKKPEVLGGAWAIDAGQAVNVLLPVCLRDRSVFGPDADEFRPERMLEENFSKLPPNSWKPFGNGERSCIGRAFAWQEAQLVVAMVLQTFDLVPDDPSYKLRIKETLTIKPDGFRVRATLRSGRTPRELCGFSNPISTNIQIKNGTIAPANDGQVGNSTPVTILYGSNSGTCEALAHRLARDAPSYGYSVTTVATLDSVIGILPRAKDELVVIITCSYDGLPADNAVRFCNWLKTLDEDALGGMPFAVFACGHHDWAKTFYKVPIMIDELLARAGAHRVAQMGKANSAVSDMFSDLENWEDEHLWSSSSAAGNETVDNVARTEVKQDITITNPRARALHHNAVECIVSETHKLSESGSALKYHVEIQLPANMKYAPGDHLSVLPINPRQNVRRALARFHLAGDSVLSVARIGHMGTLGQETLSAFDVFASYVELSQPATRRNIATLLSVTPEGEGRYELAELGGAAFESQIRDMRVSVLDLLERYKAIKLCVGAFIDMLPPLRVRTYSISSSALWKPSHASLTISVLAQPALSGQGSFLGVASNFLADLVPGDAVHFTIRPCKKQFHLPDDASAHPIIMVAAGSGIAPFRGFIQDRALQRRNGVQLQPAILFFGCRGSKQDDLYRQELDEFEAEGVVSVRRAFSGEETVSISESRMYVQDRMWADRAEVIQLWNLGAKIYVCGGINMADGVREIFNEIVGPTEVDGKRGAESLNKLSEETMSARYVAEIFSQT

SEQ ID NO: 21 - protein, 1065 aa, synthetic construct
MAIKETEQIPGPRPLPVVGNLFDMDLEHGLECLIRLADDFGPLFQITINGEKQIFATSQALVDELCDESRFHKAVMGGLEKLRMLASDGLFTAYHGERGWGIAHRILVPAFGPLRIRNMFEEMNDVAQQLCLKWARQGSSTSINITDDFTRLTLDTIALCTMNFRLNSFYNNETMHPFVKSMLYVLRESDIQSMLPGIANCIRVKARSRMSKHIQLMRNMARGIIQERRDQAEPVDDLLNTLLNGRDPVTGEGMSDDLIINNVITFLIAGHETTSGLLSFTFYYLLQNPHILERAQNEVDEVTGGERITVQHLGRLTYIDAILKESLRLMPTAPAFTVTPKKPEVLGGAWAIDAGQAVNVLLPVCLRDRSVFGPDADEFRPERMLEENFSKLPPNSWKPFGNGERSCIGRAFAWQEAQLVVAMVLQTFDLVPDDPSYKLRIKETLTIKPDGFRVRATLRNSTPPSKLVPKTAYSSSKADKSGDARSHPNMSIAGPGNNRSVSIFYGSNSGTCEALSNLLAGDFAKIGFTVQPIRTLDWAPHYFTTNELIVIVTATYDGRPTDDATEFVDWLESLQGEPLKGVSYTVFGCGNHDWATTLYKIPKFIDSTLEKHGAKRLAPLGTANVALRDPLSELEKWEEENLWPALDLALMPSNSPASLESGLKVSFQQPYTQRKEFTEATVTECYELTTSMSLTRKCHMELRLPEGVTYTAGDYLAVLPLNPMSNVQRALSRFHMAWDSVLVIESTGPTLLPTATPISVADLFSTYVELAQPATSRNLKMLASAATEQSSKQALLNLANDDSATTPSTKLPSVLDLLEAYPSLPLPIETFLEMLPPLRPRTYSISSSPSFHPSHATLTWSVVHAPSWSGHGSFYGVASNHLSDLSPGAVVRVSVRRSNPAFHPPTDPKAHPIIMIASGSGLAPFRGFIQERAWQKKAGMTLAPAMLFVGCRGKDDDLYRSAMDEFVREGVVRVFRAYSRMPEQVDAGGCMYVQERVWAERREFAELWSRGAKVFVCGGSQMSEAIKEVFANIGFAAEGEKSGGVSPDEWFRGLDPGRYATEVFN

SEQ ID NO: 22 - protein, 1064 aa, synthetic construct
MAIKETEQIPGPRPLPVVGNLFDMDLEHGLECLIRLADDFGPLFQITINGEKQIFATSQALVDELCDESRFHKAVMGGLEKLRMLASDGLFTAYHGERGWGIAHRILVPAFGPLRIRNMFEEMNDVAQQLCLKWARQGSSTSINITDDFTRLTLDTIALCTMNFRLNSFYNNETMHPFVKSMLYVLRESDIQSMLPGIANCIRVKARSRMSKHIQLMRNMARGIIQERRDQAEPVDDLLNTLLNGRDPVTGEGMSDDLIINNVITFLIAGHETTSGLLSFTFYYLLQNPHILERAQNEVDEVTGGERITVQHLGRLTYIDAILKESLRLMPTAPAFTVTPKKPEVLGGAWAIDAGQAVNVLLPVCLRDRSVFGPDADEFRPERMLEENFSKLPPNSWKPFGNGERSCIGRAFAWQEAQLVVAMVLQTFDLVPDDPSYKLRIKETLTIKPDGFRVRATLRQSRAPLTFLDGSELSTKQAHTPQEGRKSIMKTPTVGQGKYKSVSIFYGSNSGTCEALSNVFANDCAKAGFLVQPIRALDSAKEDFTSNELVVIITATYDGRPTDNATEFVDWLNSLTGKPLEGVSYAVFGCGHQDWPATLYKIPNFIDEVLEQRGAKRIAPRGAVNVAISDPFSVFVNWEEETLWPAVGLSLTPSESPISLESQLKVSFQQPYTQRKEFKEATVTKLLELSSPASLTRKCHIELRLPQGMSYTTGDYLAVLPLNPASNVQRALARLNLAWDSVLIIESAGPTQLPTATPISVADLFGAYVELSSPASSRSIKRLAAAAIDESTKDSLLRLASNDSSQLGRKQYSVLDLLEDYESIPLSVEMFLEMLPPLRPRIYSISSAPDWKPSHTTLTWSVVNAPSWSGHGSFLGVASNHLYNLSPGAVVRVSVRRSNPAFRLPQDPGAYPIILIASGSGLAPFRAFLQQRALQHKTGIVLAPALLFFGCRSKDDDLYRTELDEFESSGIVRVRRAYSKTPKEIDACGCTYVQDRIWAEKEEFRTLWDQGAVVFVCGGSKMSEAVKDVFIKIARKSSSDNCLTSFTEWFKALDSRRYVAEIFN

SEQ ID NO: 23 - protein, 1064 aa, synthetic construct
MAIKETEQIPGPRPLPVVGNLFDMDLEHGLECLIRLADDFGPLFQITINGEKQIFATSQALVDELCDESRFHKAVMGGLEKLRMLASDGLFTAYHGERGWGIAHRILVPAFGPLRIRNMFEEMNDVAQQLCLKWARQGSSTSINITDDFTRLTLDTIALCTMNFRLNSFYNNETMHPFVKSMLYVLRESDIQSMLPGIANCIRVKARSRMSKHIQLMRNMARGIIQERRDQAEPVDDLLNTLLNGRDPVTGEGMSDDLIINNVITFLIAGHETTSGLLSFTFYYLLQNPHILERAQNEVDEVTGGERITVQHLGRLTYIDAILKESLRLMPTAPAFTVTPKKPEVLGGAWAIDAGQAVNVLLPVCLRDRSVFGPDADEFRPERMLEENFSKLPPNSWKPFGNGERSCIGRAFAWQEAQLVVAMVLQTFDLVPDDPSYKLRIKETLTIKPDGFRVRATLRQSRTPHTFLNGSELTPKQTHTTQEGRKAIMKNSVLGQGKYKSVSIFYGSNSGTCEALSNVFANDCAKAGLVVQPIRALDCAKEDFTSNELVVIITATYDGRPTDNATEFVDWLTSLTGKPLGGVSYAVFGCGHQDWPATLYKIPNFIDEVLEQRGAKRIAPRGAVNVATSDPFSMFEEWEEETLWPALGLSLAPPESSTSLESGLKVSFQQPYTQRKEFKEATVTKHFELSSPASLTRKCHIELRLPQGMVYTTGDYLAVLPLNPTFNVQRALARFHLAWDSVLMIESAGPTQLPTAIPISAADLFGAYVELSSPASSRNIKRLAAAAIDESTKDSLLKLASKDSSRLGSQQRSVLDLLEEYGSIPLSVEMFLEMLPPLRPRTYSISSAPDWKPSHATLTWSVVNAPSWNGHGSFLGVASNHLYNLSPGAVVRVSVRHSNPAFRLPQDPGAYPIIMIASGSGLAPFRAFLQERALQKRAGKALAPALLFFGCRSKDDDLYRDELDEFESLGIVRVSRAYSKTPKETEARGCAYVQERIQAEEEEFRKLWDQGASIFVCGGSKMSEAVKDTFINIARKGAGNDGFRSPTEWFKALDTRRYVAEIFN

SEQ ID NO: 24 - protein, 1064 aa, synthetic construct
MAIKETEQIPGPRPLPVVGNLFDMDLEHGLECLIRLADDFGPLFQITINGEKQIFATSQALVDELCDESRFHKAVMGGLEKLRMLASDGLFTAYHGERGWGIAHRILVPAFGPLRIRNMFEEMNDVAQQLCLKWARQGSSTSINITDDFTRLTLDTIALCTMNFRLNSFYNNETMHPFVKSMLYVLRESDIQSMLPGIANCIRVKARSRMSKHIQLMRNMARGIIQERRDQAEPVDDLLNTLLNGRDPVTGEGMSDDLIINNVITFLIAGHETTSGLLSFTFYYLLQNPHILERAQNEVDEVTGGERITVQHLGRLTYIDAILKESLRLMPTAPAFTVTPKKPEVLGGAWAIDAGQAVNVLLPVCLRDRSVFGPDADEFRPERMLEENFSKLPPNSWKPFGNGERSCIGRAFAWQEAQLVVAMVLQTFDLVPDDPSYKLRIKETLTIKPDGFRVRATLRQSRAPLAFLNGSELSPKQAHNTQEGRKSIIEKSILGQGKYKSVSIFYGSNSGTCEALSNMFANDCAKAGFIVQPIRALNSAKEDFTSNELVVIITATYDGRPTDNATEFVDWLNSLTGKPLEGVSYAVFGCGHQDWPATLYKIPIFVDEVLEQRGAKRIAPRGAVNVATSEPFSVFEDWEEETLWPALGLSLTPSESSTSLESGLKVSFQQPYTQRKGFKEATVTKHLELSSPASLTRKCHIELRLPQGMSYTTGDYLAVLPMNPTANVQRALARFHLAWDSVLIIESAGTTQLPTATPISVADLFGAYVELSSPASSRNIKRLAAAAIDEPTKHSLLKLANNESSRLRGKQQSVLDLLEDYESIPLSVEMFLEMLLPLRPRTYSISSAPDWKPSHATLTWSVLDAPSWSGHGSFLGVASNHLYNLSPGAVVRVSVQRSNPAFRLPQDPGAYPIIMIASGSGLAPFRAFLEERALKHRTGKALAPALLFFGCRSKDDDLYRNELDEFESSGIVRVRRAYSKTPKETDACGCTYVQDRIRAEKEEFRKLWEQGASIFVCGGSKMSEAVKDLFINIARKSAGNDGFRSSAEWFKALDSRRYVAEIFN

SEQ ID NO: 25 - protein, 1068 aa, synthetic construct
MAIKETEQIPGPRPLPVVGNLFDMDLEHGLECLIRLADDFGPLFQITINGEKQIFATSQALVDELCDESRFHKAVMGGLEKLRMLASDGLFTAYHGERGWGIAHRILVPAFGPLRIRNMFEEMNDVAQQLCLKWARQGSSTSINITDDFTRLTLDTIALCTMNFRLNSFYNNETMHPFVKSMLYVLRESDIQSMLPGIANCIRVKARSRMSKHIQLMRNMARGIIQERRDQAEPVDDLLNTLLNGRDPVTGEGMSDDLIINNVITFLIAGHETTSGLLSFTFYYLLQNPHILERAQNEVDEVTGGERITVQHLGRLTYIDAILKESLRLMPTAPAFTVTPKKPEVLGGAWAIDAGQAVNVLLPVCLRDRSVFGPDADEFRPERMLEENFSKLPPNSWKPFGNGERSCIGRAFAWQEAQLVVAMVLQTFDLVPDDPSYKLRIKETLTIKPDGFRVRATLREGWTARKVEQHLNRSSDLETSTAKAHPTPTAPTTQDSGHPITILYGSNSGTCEALAHTLAADARGHGFLPTTVDTLDSAKQNLPADQPIVIITASYEGQPTDNAGHFYNWLQNVTELKTSYAVFGCGHSDWRQSFHRIPASIDSLLEKAGSTRICKMGSADAAKGDMISDFNGWEDETLWPALQKQYTCTDSETSSPSHGQSLSVEVFSKRASHLRSDVSEARVVSAKVLSAPGVAPKRHIELQLPSDMTYRAGDYLAVLPLNPAETVHRVLARFSLPWDAMLSITSKDHTTLPTEHPVSAQDLFSAYLELSQPATKRNVTMLIEVSHEEAIKEELKKLSDDFDAEITAKRVSLLDLVERFPTIALPLSAFISSLISMRVRQYSISSSPLSDQNRATLTYAVLDEPSTSGQGRHIGVASNYLSQLKPGDIVHVAVKPSHASFHLPSSPETTPIIMIAAGAGLAPFRGYIQERASQIQSGRTLAPAHLYFGCRYPDKDDLYRDEIDSWEQIGAVVAHRCFSQAPEQSGGHKHVDGLILQDKELLSELWEGGAKVYVCGSRGLGDSVKKAFIKLAKEKAVADGNKNAEDDAKLEKWFESLRNERFATDVFT

SEQ ID NO: 26 - protein, 1070 aa, synthetic construct
MAIKETEQIPGPRPLPVVGNLFDMDLEHGLECLIRLADDFGPLFQITINGEKQIFATSQALVDELCDESRFHKAVMGGLEKLRMLASDGLFTAYHGERGWGIAHRILVPAFGPLRIRNMFEEMNDVAQQLCLKWARQGSSTSINITDDFTRLTLDTIALCTMNFRLNSFYNNETMHPFVKSMLYVLRESDIQSMLPGIANCIRVKARSRMSKHIQLMRNMARGIIQERRDQAEPVDDLLNTLLNGRDPVTGEGMSDDLIINNVITFLIAGHETTSGLLSFTFYYLLQNPHILERAQNEVDEVTGGERITVQHLGRLTYIDAILKESLRLMPTAPAFTVTPKKPEVLGGAWAIDAGQAVNVLLPVCLRDRSVFGPDADEFRPERMLEENFSKLPPNSWKPFGNGERSCIGRAFAWQEAQLVVAMVLQTFDLVPDDPSYKLRIKETLTIKPDGFRVRATLRDGLTPTSLSARLAGTPNTGQEDNSAQNGGVEESASGPGQPISVFYGSNSGTCETLARRIAADAPRHGFKVTAVDCMDVANGNIPKDQPVVFLTASYEGEPPDNARHFVSYLENLEDKKALEGVNYAVFGCGHHDWTSTFYRIPKLVDSALNENGATRLAESGFADAGGADVFVAFETWEDSTFWPALKEKYGIETANGANDEQLSLTVAVTNPRSEVLHTDVQLAQVIETRILTADGEPIKKHIEIKLPSDSSYRSGDYLAVLPINPRETVARAMRRFELPWDSHLEIEGGGHVDLPTEKSLPAFDIFGSYVELAQPATKRNILALAASTEDEADKAMLKDLADASYEKEILEKRMSVLDLLETYPSAKLSLGSFLAMLPRMRIRQYSISSSPLWKPNHVTLTFSLLNEPSKSGHGSYVGVATSYLASLQAGDKLPVSIKQSHASFHLPQDQGSTPIICVAAGTGLAPFRGFIQERAALIEAGRKVAPLVLYYGCREPGRDDLYADELAKWEKLGAVKVKHTYSRTPDKSDGNKYVQEAIWADRREVGELWENGAKVFVCGSKRLSTGVQDVAIRMTRAKGKLVGKEVSEEEAFKWWQELRNVRYATDVFE

SEQ ID NO: 27 - protein, 1069 aa, synthetic construct
MAIKETEQIPGPRPLPVVGNLFDMDLEHGLECLIRLADDFGPLFQITINGEKQIFATSQALVDELCDESRFHKAVMGGLEKLRMLASDGLFTAYHGERGWGIAHRILVPAFGPLRIRNMFEEMNDVAQQLCLKWARQGSSTSINITDDFTRLTLDTIALCTMNFRLNSFYNNETMHPFVKSMLYVLRESDIQSMLPGIANCIRVKARSRMSKHIQLMRNMARGIIQERRDQAEPVDDLLNTLLNGRDPVTGEGMSDDLIINNVITFLIAGHETTSGLLSFTFYYLLQNPHILERAQNEVDEVTGGERITVQHLGRLTYIDAILKESLRLMPTAPAFTVTPKKPEVLGGAWAIDAGQAVNVLLPVCLRDRSVFGPDADEFRPERMLEENFSKLPPNSWKPFGNGERSCIGRAFAWQEAQLVVAMVLQTFDLVPDDPSYKLRIKETLTIKPDGFRVRATLRNGISATSLERALAYATIKDLPPATGAQQTRRGTIAKGKPIAIYYGSNTGTCEALANRLASDAANHGFRADVVDTLDSVRENLPTDKPIVIVTASYEGQPTDNAAHFVNWIGTLKEKELEKVTYAVFGCGHHDWAKTFHRIPKYLDATLEERGAIRLVGMGSADAAHGDIFTDFESWEDQVLWPTLRQKYGSSEAEGEGSLEATLDVEISTPRSTILRQDVREARVEEVAVLSASGVAEKRHIEISLPSDMSYSAGDYLAILPLNPKENIRRATKYFGLSWDSMLTISSAGPTTLPTDTPISAIDVFGAYIELAQPASKRNLLALADATRDDKTKNELTRLAEETFAEEITAKRVSVIDLLERFPSVQLPLGVFLKMQPPMRVRQYSISSSPLWNLNSVTLTYSVVNQPALSGQGRYVGVASNYLSNLVPGDKLHVSVRSSHQAFHLPKDAKNVPVIMMAAGTGLAPFRGFIQERAAQLAAGRSLAPALLFYGCRSPESDLMYPELLKRWEQLGAVTLRFAFSCGPQQSEGCKYVQDRVYYDRSEIVELFDAGAKLFVCGSREVGEGIQETLIKIAKERRKEVDGKEVDDTKAREWFEGLRNERFATDVFA

SEQ ID NO: 28 - protein, 1067 aa, synthetic construct
MAIKETEQIPGPRPLPVVGNLFDMDLEHGLECLIRLADDFGPLFQITINGEKQIFATSQALVDELCDESRFHKAVMGGLEKLRMLASDGLFTAYHGERGWGIAHRILVPAFGPLRIRNMFEEMNDVAQQLCLKWARQGSSTSINITDDFTRLTLDTIALCTMNFRLNSFYNNETMHPFVKSMLYVLRESDIQSMLPGIANCIRVKARSRMSKHIQLMRNMARGIIQERRDQAEPVDDLLNTLLNGRDPVTGEGMSDDLIINNVITFLIAGHETTSGLLSFTFYYLLQNPHILERAQNEVDEVTGGERITVQHLGRLTYIDAILKESLRLMPTAPAFTVTPKKPEVLGGAWAIDAGQAVNVLLPVCLRDRSVFGPDADEFRPERMLEENFSKLPPNSWKPFGNGERSCIGRAFAWQEAQLVVAMVLQTFDLVPDDPSYKLRIKETLTIKPDGFRVRATLRNHMSATALERALASATISEPTGKTETGGAQKKAAQGKPLAIYYGSNSGTCEALANRLAGGAANHGFRVETIDILDSVKENLPADKPVAIVTASYEGQPADNAAHFVTWLDSLKASELNNVSYAVFGCGHRDWASTFLKVPKYIDAAIEERGGTRLVGMGSADASHGDIFTEFEAWEDKVFWPALKERYGTSDAVDGGPFEASLDVDVSTPRSSMLRQDVREAKVEAVEVLSAPGAPEKRHIEISLPSDMNYSAGDYLAILPLNPKENVARAMRYFGLAWDSVLTINSSGPTTLPVDTPCPASDVLGAYVELAQPATKRDIAALIEATPKEEEKKELTRLVEDVFATEITEKRVSVLDLLERFPSVQLPLGPFLKMLPPMRVRQYSISSSPLWNPDNVTLTYAVVDQPAFSGQGRFVGVASSYLSSLAPGDKLHVSVRSSHQAFHLPKDAKNVPVIMVAAGTGLAPFRGFIQERAAQLAAGRPLAPALLFYGCHSPDTDLLYSDLLKKWEGMGAVDIRPAYSRAADQSEDCKHVQDRLYHDRKDVVELWDKGAKVFVCGSREVGKGVEDACLKIAVEERRAKGKEGDEEKVGKWWEGLRNERFATDVFA

SEQ ID NO: 29 - protein, 1067 aa, synthetic construct
MAIKETEQIPGPRPLPVVGNLFDMDLEHGLECLIRLADDFGPLFQITINGEKQIFATSQALVDELCDESRFHKAVMGGLEKLRMLASDGLFTAYHGERGWGIAHRILVPAFGPLRIRNMFEEMNDVAQQLCLKWARQGSSTSINITDDFTRLTLDTIALCTMNFRLNSFYNNETMHPFVKSMLYVLRESDIQSMLPGIANCIRVKARSRMSKHIQLMRNMARGIIQERRDQAEPVDDLLNTLLNGRDPVTGEGMSDDLIINNVITFLIAGHETTSGLLSFTFYYLLQNPHILERAQNEVDEVTGGERITVQHLGRLTYIDAILKESLRLMPTAPAFTVTPKKPEVLGGAWAIDAGQAVNVLLPVCLRDRSVFGPDADEFRPERMLEENFSKLPPNSWKPFGNGERSCIGRAFAWQEAQLVVAMVLQTFDLVPDDPSYKLRIKETLTIKPDGFRVRATLRHGMTPTELEHVLAGNGATSSSTHNIKAAANSDTKAGGSGKPMAIFYGSNSGTCEALANRLASDAPGHGFSATTVGPLDQAKQNLPEDRPVVIVTASYEGQPPSNAAHFIKWMEDLNGNEMEKVSYAVFACGHHDWVETFHRIPKLVDSTLEKRGGTRLVPMGSADAATSDMFSDFEAWEDTVLWPGLKEKYKISDEESGGQKGLLVEVSTPRKTSLRQDVEEALVVAEKTLTKSGPAKKHIEIQLPSAMTYKAGDYLAILPLNPKSTVARVFRRFSLAWDSFLKIQSEGPTTLPTNVAISAFDVFSAYVELSQPATKRNILALAEATEDKATIQELERLAGDAYQAEISPKRVSVLDLLEKFPAVALPISSYLAMLPPMRVRQYSISSSPFADPSKLTLTYSLLDAPSLSGQGRHVGVATNFLSHLTAGDKLHVSVRASSEAFHLPSDAEKTPIICVAAGTGLAPFRGFIQERAAMLAAGRTLAPALLFFGCRNPEIDDLYAEEFERWEKMGAVDVRRAYSRATDKSEGCKYVQDRVYHDRADVFKVWDQGAKVFICGSREIGKAVEDVCVRLAIEKAQQNGRDVTEEMARAWFERSRNERFATDVFD

SEQ ID NO: 30 - protein, 1081 aa, synthetic construct
MAIKETEQIPGPRPLPVVGNLFDMDLEHGLECLIRLADDFGPLFQITINGEKQIFATSQALVDELCDESRFHKAVMGGLEKLRMLASDGLFTAYHGERGWGIAHRILVPAFGPLRIRNMFEEMNDVAQQLCLKWARQGSSTSINITDDFTRLTLDTIALCTMNFRLNSFYNNETMHPFVKSMLYVLRESDIQSMLPGIANCIRVKARSRMSKHIQLMRNMARGIIQERRDQAEPVDDLLNTLLNGRDPVTGEGMSDDLIINNVITFLIAGHETTSGLLSFTFYYLLQNPHILERAQNEVDEVTGGERITVQHLGRLTYIDAILKESLRLMPTAPAFTVTPKKPEVLGGAWAIDAGQAVNVLLPVCLRDRSVFGPDADEFRPERMLEENFSKLPPNSWKPFGNGERSCIGRAFAWQEAQLVVAMVLQTFDLVPDDPSYKLRIKETLTIKPDGFRVRATLRHGMSPTELEHRLMGRGLNKTRLPSPLSAQTNGHQTNGSKESNVGGIPISIYYGSNSGTCEALAQRLATDATTHGFHATTVEPLDTARENLPKDRPMVIITASYEGQPPENAAHFVAWLESLKAQELSNVPYAVFGCGHHDWVQTFHRIPKLVNTIMEERGANRVAPMGLTNAADRDMFSDFEAWEDKVLWPALTEKYDVTEARKANGSDSGLVVQISYPRTLALRQDVEEAVVVEEKTLTAPGAAQKKHMDIQLPPGMTFRAGDYLAVLPLNPREHVERVFHRFQLARDTCLTIFGDKRTALPVNQTISAYDILSAYVELAQPATKRNVLSLLEATKDAETIEKLERLASAEYQAEITQKRVTILDLLERFPSITLPIGSFLAMLPPMRVRQYSISSSPLRNPSQATLTYSILGGPSLSGQGRYLGVATSYLASLKAGDKLHVAVRPSHVAFHLPHQPEKTPLILVAAGSGIAPFRGFIQERAAMIAAGRTVAPALLFYGCREPGIDDLYRQELDAWEGMGAVTVRRAYSRAAEQSEGCRYVQHRLYHDKKDVAGLWAKGAKLFVCGTRDVGKAVEEACVRVILESAKEGGGQPELRDLDYEGAKKWFEGIRNERYATDVFD

SEQ ID NO: 31 - protein, 1076 aa, synthetic construct
MAIKETEQIPGPRPLPVVGNLFDMDLEHGLECLIRLADDFGPLFQITINGEKQIFATSQALVDELCDESRFHKAVMGGLEKLRMLASDGLFTAYHGERGWGIAHRILVPAFGPLRIRNMFEEMNDVAQQLCLKWARQGSSTSINITDDFTRLTLDTIALCTMNFRLNSFYNNETMHPFVKSMLYVLRESDIQSMLPGIANCIRVKARSRMSKHIQLMRNMARGIIQERRDQAEPVDDLLNTLLNGRDPVTGEGMSDDLIINNVITFLIAGHETTSGLLSFTFYYLLQNPHILERAQNEVDEVTGGERITVQHLGRLTYIDAILKESLRLMPTAPAFTVTPKKPEVLGGAWAIDAGQAVNVLLPVCLRDRSVFGPDADEFRPERMLEENFSKLPPNSWKPFGNGERSCIGRAFAWQEAQLVVAMVLQTFDLVPDDPSYKLRIKETLTIKPDGFRVRATLRHGLTPTQLEHRLNGTTAPTTPIENAASGLSLGGDQKANAAKGKPMAIYYGSNSGTCEALARRLATDAPSHGFNATVVDSLDAAHQNIPTDKPVVVVTASYEGEPPDNAALFCNWLQALKDKELENVNYAVFGCGHHDWSQTFHKIPKMVDATIEKHGGTRIAALGVADAAEGDMFTKFETWEDELLWPALREKYGVEDSVQDGESLQGGLSVEISTPRSSTLRQDVKEAVVIETRDLTSPEAPTKRHIEIQLPSDMTYTAGDYLAILPINPKAVVHRALRRFRLAWDAHVTINSNSTTTLPTGYPVPASDVLGAYVELGQPATKRGILALAEATKDEDAKAALQRLAGDDYVTEVNAKRRSVLDLLEQFPAVELPFATFLALLPPMRVRQYSISSSPLWHQNHLTLTYAVLDAPALSGTGQRYLGVATSYLSSLAEGDKLHVAIRPSHASFHLPVDTEKTPIMCIAAGSGLAPFRGFIQERAAMLDAGRTLAPALLFFGCRHPDQDDLYREELDRWQAMGAVDVRRAYSRDPASSEGCAHVQDRLWNDRAEVQALWDRGAKVFVCGSREVGDAVREVAIRMYVDGAKVNHGRDLSVEEATEKFDAMRNERYASDVFD

SEQ ID NO: 32 - protein, 1071 aa, synthetic construct
MAIKETEQIPGPRPLPVVGNLFDMDLEHGLECLIRLADDFGPLFQITINGEKQIFATSQALVDELCDESRFHKAVMGGLEKLRMLASDGLFTAYHGERGWGIAHRILVPAFGPLRIRNMFEEMNDVAQQLCLKWARQGSSTSINITDDFTRLTLDTIALCTMNFRLNSFYNNETMHPFVKSMLYVLRESDIQSMLPGIANCIRVKARSRMSKHIQLMRNMARGIIQERRDQAEPVDDLLNTLLNGRDPVTGEGMSDDLIINNVITFLIAGHETTSGLLSFTFYYLLQNPHILERAQNEVDEVTGGERITVQHLGRLTYIDAILKESLRLMPTAPAFTVTPKKPEVLGGAWAIDAGQAVNVLLPVCLRDRSVFGPDADEFRPERMLEENFSKLPPNSWKPFGNGERSCIGRAFAWQEAQLVVAMVLQTFDLVPDDPSYKLRIKETLTIKPDGFRVRATLRNHMTATTLERALASTTISAAEAAKSKGQTVSKGPKAQGKPMSILYGSNTGTCEALANRLASDAANHGFHAQVVDTLDSAKEKLPTDRPVVIVTASYEGNPADNAAHFVNWIGGLKSNELNDVSYAVFGCGHRDWVKTFHRIPKYLDSVIEKRGGTRIAPIGTTDAAAGDIFTDFETWEDQVFWPAMREKYGTAEGSTDGSLETSMDVEISSPRSSTLRQDVREARVEEVRTLSAPGSPEKRHIEISLPSDMTYSAGDYLAILPINPKESIARAMRYFGLPWDSILTLSASAGPTTLPTGQPIAAADMFGAYVELAQPASKRNILALIEASTDEKTKEALTNLSDSAFTTEITQKRISVLDLLERFPSVQLPLGAFLKMMPPMRVRQYSISSSPLWNSNSVTLTYSVVDQPAMSGQGRFVGVASNYLSSLAPGDKLHVSVRQSHQSFHLPKDSKNVPVIMIAAGSGLAPFRGFIQERAAQIGAGRPLAPALLFYGCHSSDTDSLYADEIARWEKMGAVSARYTFSRAPEQSEGCKYVQDRLYHDRKDVVELFDRGAKLFVCGSRAVGEGVQNTCVRIVKEQSEEVHGCDVSEKKARAWFDGLRNERFATDVFA

SEQ ID NO: 33 - protein, 1071 aa, synthetic construct
MAIKETEQIPGPRPLPVVGNLFDMDLEHGLECLIRLADDFGPLFQITINGEKQIFATSQALVDELCDESRFHKAVMGGLEKLRMLASDGLFTAYHGERGWGIAHRILVPAFGPLRIRNMFEEMNDVAQQLCLKWARQGSSTSINITDDFTRLTLDTIALCTMNFRLNSFYNNETMHPFVKSMLYVLRESDIQSMLPGIANCIRVKARSRMSKHIQLMRNMARGIIQERRDQAEPVDDLLNTLLNGRDPVTGEGMSDDLIINNVITFLIAGHETTSGLLSFTFYYLLQNPHILERAQNEVDEVTGGERITVQHLGRLTYIDAILKESLRLMPTAPAFTVTPKKPEVLGGAWAIDAGQAVNVLLPVCLRDRSVFGPDADEFRPERMLEENFSKLPPNSWKPFGNGERSCIGRAFAWQEAQLVVAMVLQTFDLVPDDPSYKLRIKETLTIKPDGFRVRATLRNHMTPTTLERALSSTSIGESAAANTGKPKPQTANNSVTQGQPIRIYYGSNTGTCEALAQRLASDASAHGFTADLVDTLDSAKESLPTDKPVIIVTASYEGQPTDNAAHYVNWLGGLKGSELEKVPFAVFGCGHRDWAKTFLKIPKFVDEIMEQRGGTRLVPMGSADAAGGDIFTEFETWEDQVLWPALREKYSSADTSGGSSYDTSLQVEISTPRSSMLRQDVKEARVEEVEILSKPGVPEKRHIEISLPSDMTYSAGDYLAILPINPKNNVTRAMRHFGLAWDSVLTISSVGPTTLPTNAPISAADVFGAYVELGQPASKRNLQALIEATEDEGAKKDLTELSDISFATEITGKRVSVLDLLERFPSVELPLGHFLKMLPPMRVRQYSISSSPLWNPNHVTLTYAVVDQPALSGQGRFVGVASSYLSSLAAGDKLHVSVRTSHQAFHLPKDTKNVPVIMIAAGTGLAPFRGFIQERAAQIAAGRSLAPALLFYGCRSPDSDALYSSLLQRWETLSAVSVRYSYSRAADQSEGCKYVQDRLYHDRKEVVELFEKGAKIFICGGREVGEGVQNICMKIMREETESMGKDVSEQRAKEWFEGLRNERFATDVFA

SEQ ID NO: 34 - protein, 1072 aa, synthetic construct
MAIKETEQIPGPRPLPVVGNLFDMDLEHGLECLIRLADDFGPLFQITINGEKQIFATSQALVDELCDESRFHKAVMGGLEKLRMLASDGLFTAYHGERGWGIAHRILVPAFGPLRIRNMFEEMNDVAQQLCLKWARQGSSTSINITDDFTRLTLDTIALCTMNFRLNSFYNNETMHPFVKSMLYVLRESDIQSMLPGIANCIRVKARSRMSKHIQLMRNMARGIIQERRDQAEPVDDLLNTLLNGRDPVTGEGMSDDLIINNVITFLIAGHETTSGLLSFTFYYLLQNPHILERAQNEVDEVTGGERITVQHLGRLTYIDAILKESLRLMPTAPAFTVTPKKPEVLGGAWAIDAGQAVNVLLPVCLRDRSVFGPDADEFRPERMLEENFSKLPPNSWKPFGNGERSCIGRAFAWQEAQLVVAMVLQTFDLVPDDPSYKLRIKETLTIKPDGFRVRATLRTGMSATTLERALGSASINAPSAGTQSAKSHAPTKTSGKKGKPLAIYYGSNTGTCEALAQRLAGDAANHGFSAETVDILDTAKEKLPTDKPVVIITASYEGQPADNAAHFVKWLEALEGSELENVSYSVFGCGHHDWSTTFHKIPKLIDNGLEKRGAKRIAPIGTADAATGDIFSDFETWEDRVFWPAVSEQYGVEADDSASTYESSINVEVSSPRSSTLRQDVREARVEETKVLTAPGAPEKRHIEISLPSDMTYNAGDYLAILPLNPKENVNRAMRYFGLAWDSMLTIQSTGPSTLPMDAPIPASDLFGAYIELAQPASRRNINSLIEATQDEETKAELTRLAGDAFPEEIISKRVSVLDLLERFPSIQLPLGPFLQMQPPMRVRQYSISSSPLWNPSHVTLTYAVLDQPAYSGHGRFVGVASNYLSTLSPGDVLHVSVRSSHKAFHLPKDGQNVPIICIAAGSGIAPFRGFIQERAAQLAAGRTLAPALLFFGCHSPTTDSLYSAELSRWESLGAVSVRHAFSRAPDQSLGCKYVQDRLWHDRKEVVELFERGAKIFTCGSKKVGEGVAETCIRIAKESVKESEGREADDRKAREWFEGLRNERFATDVFA

SEQ ID NO: 35 - protein, 1062 aa, synthetic construct
MAIKETEQIPGPRPLPVVGNLFDMDLEHGLECLIRLADDFGPLFQITINGEKQIFATSQALVDELCDESRFHKAVMGGLEKLRMLASDGLFTAYHGERGWGIAHRILVPAFGPLRIRNMFEEMNDVAQQLCLKWARQGSSTSINITDDFTRLTLDTIALCTMNFRLNSFYNNETMHPFVKSMLYVLRESDIQSMLPGIANCIRVKARSRMSKHIQLMRNMARGIIQERRDQAEPVDDLLNTLLNGRDPVTGEGMSDDLIINNVITFLIAGHETTSGLLSFTFYYLLQNPHILERAQNEVDEVTGGERITVQHLGRLTYIDAILKESLRLMPTAPAFTVTPKKPEVLGGAWAIDAGQAVNVLLPVCLRDRSVFGPDADEFRPERMLEENFSKLPPNSWKPFGNGERSCIGRAFAWQEAQLVVAMVLQTFDLVPDDPSYKLRIKETLTIKPDGFRVRATLRHGMTPTELEQHLAGKLSSEHYHAMSRKTSSNISASAGKPLAVFYGSNSGTCEALAQRIAADAPRHGFRASTVAPLDDANQKIPKDRPVVIVTASYEGQPPSNAALFVAWMESLKGKEMENVSYAVFGCGHRDWVQTFHRIPKLVDSKLGELGGQRIVPLATTDAAERDMFSDFEAWEDESLWPALQKMYGTEDSGDSNGDGINVEVSLPRKSTLRQDVEEALVVSSKTLTDSGSVKKHIEIQLPTGMTYRAGDYLAVLPFNPKKTVSRVFRRFKLSWDATLKISSDRPTTLPTDTAISAADVLAAYVELSQPATKRNIQALADATQDQAVVQKLQKLAGEDYHENITVKRISILDLLEQFPTIALPFGSYLGMLPPMRVRQYSISSSPLADPAKVTLTYSVLEQPALSGQGAHVGVATNFLAGLVPGERLHVAVRPSKSFHLPSDAENTPIVCVAAGSGMAPFRGFMQERAAMIGAGRKVAPALLFFGCRNPDKDDLYADELARWEELGAVEVRRAYSRVPEKSFGCKYVQHRLSHDREEVYKLWDRGAKIYVCGSRDVGKAVEAVCIDMVQESGSSKDVTEDKAKAWFEKQRNERYVTDVFD

SEQ ID NO: 36 - protein, 1085 aa, synthetic construct
MAIKETEQIPGPRPLPVVGNLFDMDLEHGLECLIRLADDFGPLFQITINGEKQIFATSQALVDELCDESRFHKAVMGGLEKLRMLASDGLFTAYHGERGWGIAHRILVPAFGPLRIRNMFEEMNDVAQQLCLKWARQGSSTSINITDDFTRLTLDTIALCTMNFRLNSFYNNETMHPFVKSMLYVLRESDIQSMLPGIANCIRVKARSRMSKHIQLMRNMARGIIQERRDQAEPVDDLLNTLLNGRDPVTGEGMSDDLIINNVITFLIAGHETTSGLLSFTFYYLLQNPHILERAQNEVDEVTGGERITVQHLGRLTYIDAILKESLRLMPTAPAFTVTPKKPEVLGGAWAIDAGQAVNVLLPVCLRDRSVFGPDADEFRPERMLEENFSKLPPNSWKPFGNGERSCIGRAFAWQEAQLVVAMVLQTFDLVPDDPSYKLRIKETLTIKPDGFRVRATLRDGLNPTTLERRLQGLGQGDALSTAPANNGAGPGSADATGAPLTVLYGSNSGTCEALARRIATDAPAHGFHAATVDCMDSAKGKLPTDQAVVIITASYEGQPPDNAGHFVAWLEGSDKTTRPFKDVSYAVFGCGHKDWAQTFHRVPRLVDDSLQELGASRLVEPGLSDVSTGAVFSDFESWEDEKLWPALASRYQVSSKASAAENGAEGSAVKVHVSNPRISTLRQDVKEARVIEAKALTSGPESTTDGGRQFKRHLEIELPEGMEYSAGDYLAVLPVNPIDTVLRTLHRFGLARDAHVTIEMSSSQSTALPTNTSVPAFELLSSYVELSQPATKRNLSALQDAASDAETKSQLDKLSGDDFVDEVGAKRVSVLDLLERFPTIDLSIGSFLSMMPPMRVRQYSISSSPLVNPSRATLSFSVLDAPSLSGQGRHIGVASNYLSSLTAGDSLQVSVRPSHAAFSLPSDPERTPLICAAAGTGLAPFRGFAQERAAMISAGRKLAPALLYFGCRAPDLDDLYRDEFDAWERLGAVEVRRTYSRRPEASDGCRHVQDRILADADRLLGLWRDGAKLYVCGSRGVSQSVMDAMTRIKMDKDRELHGKETTAEEARQYFDDLRNVRYATDVFD

SEQ ID NO: 37 - protein, 1067 aa, synthetic construct
MAIKETEQIPGPRPLPVVGNLFDMDLEHGLECLIRLADDFGPLFQITINGEKQIFATSQALVDELCDESRFHKAVMGGLEKLRMLASDGLFTAYHGERGWGIAHRILVPAFGPLRIRNMFEEMNDVAQQLCLKWARQGSSTSINITDDFTRLTLDTIALCTMNFRLNSFYNNETMHPFVKSMLYVLRESDIQSMLPGIANCIRVKARSRMSKHIQLMRNMARGIIQERRDQAEPVDDLLNTLLNGRDPVTGEGMSDDLIINNVITFLIAGHETTSGLLSFTFYYLLQNPHILERAQNEVDEVTGGERITVQHLGRLTYIDAILKESLRLMPTAPAFTVTPKKPEVLGGAWAIDAGQAVNVLLPVCLRDRSVFGPDADEFRPERMLEENFSKLPPNSWKPFGNGERSCIGRAFAWQEAQLVVAMVLQTFDLVPDDPSYKLRIKQTLTIKPDGFRVRATLRHGMTPTELEHVLAGNGATSSSTHNIKAAANSDTKAGGSGKPMAIFYGSNSGTCEALANRLASDAPGHGFSATTVGPLDQAKQNLPEDRPVVIVTASYEGQPPSNAAHFIKWMEDLNGNEMEKVSYAVFACGHHDWVETFHRIPKLVDSTLEKRGGTRLVPMGSADAATSDMFSDFEAWEDTVLWPGLKEKYKISDEESGGQKGLLVEVSTPRKTSLRQDVEEALVVAEKTLTKSGPAKKHIEIQLPSAMTYKAGDYLAILPLNPKSTVARVFRRFSLAWDSFLKIQSEGPTTLPTNVAISAFDVFSAYVELSQPATKRNILALAEATEDKATIQELERLAGDAYQAEISPKRVSVLDLLEKFPAVALPISSYLAMLPPMRVRQYSISSSPFADPSKLTLTYSLLDAPSLSGQGRHVGVATNFLSHLTAGDKLHVSVRASSEAFHLPSDAEKTPIICVAAGTGLAPFRGFIQERAAMLAAGRTLAPALLFFGCRNPEIDDLYAEEFERWEKMGAVDVRRAYSRATDKSEGCKYVQDRVYHDRADVFKVWDQGAKVFICGSREIGKAVEDVCVRLAIEKAQQNGRDVTEEMARAWFERSRNERFATDVFD

SEQ ID NO: 38 - protein, 1067 aa, synthetic construct
MAIKETEQIPGPRPLPVVGNLFDMDLEHGLECLIRLADDFGPLFQITINGEKRIFATSQALVDELCDESRFHKAVMGGLEKLRMLASDGLFTAYHGERGWGIAHRILVPAFGPLRIRNMFEEMNDVAQQLCLKWARQGSSTSINITDDFTRLTLDTIALCTMNFRLNSFYNNETMHPFVKSMLYVLRESDIQSMLPGIANCIRVKARSRMSKHIQLMRNMARGIIQERRDQAEPVDDLLNTLLNGRDPVTGEGMSDDLIINNVITFLIAGHETTSGLLSFTFYYLLQNPHILERAQNEVDEVTGGERITVQHLGRLTYIDAILKESLRLMPTAPAFTVTPKKPEVLGGAWAIDAGQAVNVLLPVCLRDRSVFGPDADEFRPERMLEENFSKLPPNSWKPFGNGERSCIGRAFAWQEAQLVVAMVLQTFDLVPDDPSYKLRIKQTLTIKPDGFRVRATLRHGMTPTELEHVLAGNGATSSSTHNIKAAANSDTKAGGSGKPMAIFYGSNSGTCEALANRLASDAPGHGFSATTVGPLDQAKQNLPEDRPVVIVTASYEGQPPSNAAHFIKWMEDLNGNEMEKVSYAVFACGHHDWVETFHRIPKLVDSTLEKRGGTRLVPMGSADAATSDMFSDFEAWEDTVLWPGLKEKYKISDEESGGQKGLLVEVSTPRKTSLRQDVEEALVVAEKTLTKSGPAKKHIEIQLPSAMTYKAGDYLAILPLNPKSTVARVFRRFSLAWDSFLKIQSEGPTTLPTNVAISAFDVFSAYVELSQPATKRNILALAEATEDKATIQELERLAGDAYQAEISPKRVSVLDLLEKFPAVALPISSYLAMLPPMRVRQYSISSSPFADPSKLTLTYSLLDAPSLSGQGRHVGVATNFLSHLTAGDKLHVSVRASSEAFHLPSDAEKTPIICVAAGTGLAPFRGFIQERAAMLAAGRTLAPALLFFGCRNPEIDDLYAEEFERWEKMGAVDVRRAYSRATDKSEGCKYVQDRVYHDRADVFKVWDQGAKVFICGSREIGKAVEDVCVRLAIEKAQQNGRDVTEEMARAWFERSRNERFATDVFD

SEQ ID NO: 39 - protein, 1067 aa, synthetic construct
MAIKETEQIPGPRPLPVVGNLFDMDLEHGLECLIRLADDFGPLFQITINGEKRIFATSQALVDELCDESRFHKAVMGGLEKLRMLASDGLFTAYHGERGWGIAHRILVPAFGPLRIRNMFEEMNDVAQQLCLKWARQGSSTSINITDDFTRLTLDTIALCTMNFRLNSFYNNETMHPFVKSMLYVLRESDIQSMLPGIANCIRVKARSRMSKHIQLMRNMARGIIQERRDQAEPVDDLLNTLLNGRDPVTGEGMSDDLIINNVITFLIAGHETTSGLLSFTFYYLLQNPHILERAQNEVDEVTGGERITVQHLGRLTYIDAILKESLRLMPTAPAFTVTPKKPEVLGGAWAIDAGQAVNVLLAVCLRDRSVFGPDADEFRPERMLEENFSKLPPNSWKPFGNGERSCIGRAFAWQEAQLVVAMVLQTFDLVPDDPSYKLRIKQTLTIKPDGFRVRATLRHGMTPTELEHVLAGNGATSSSTHNIKAAANSDTKAGGSGKPMAIFYGSNSGTCEALANRLASDAPGHGFSATTVGPLDQAKQNLPEDRPVVIVTASYEGQPPSNAAHFIKWMEDLNGNEMEKVSYAVFACGHHDWVETFHRIPKLVDSTLEKRGGTRLVPMGSADAATSDMFSDFEAWEDTVLWPGLKEKYKISDEESGGQKGLLVEVSTPRKTSLRQDVEEALVVAEKTLTKSGPAKKHIEIQLPSAMTYKAGDYLAILPLNPKSTVARVFRRFSLAWDSFLKIQSEGPTTLPTNVAISAFDVFSAYVELSQPATKRNILALAEATEDKATIQELERLAGDAYQAEISPKRVSVLDLLEKFPAVALPISSYLAMLPPMRVRQYSISSSPFADPSKLTLTYSLLDAPSLSGQGRHVGVATNFLSHLTAGDKLHVSVRASSEAFHLPSDAEKTPIICVAAGTGLAPFRGFIQERAAMLAAGRTLAPALLFFGCRNPEIDDLYAEEFERWEKMGAVDVRRAYSRATDKSEGCKYVQDRVYHDRADVFKVWDQGAKVFICGSREIGKAVEDVCVRLAIEKAQQNGRDVTEEMARAWFERSRNERFATDVFD

SEQ ID NO: 40 - protein, 1067 aa, synthetic construct
MAIKETEQIPGPRPLPVVGNLFDMDLEHGLECLIRLADDFGPLFQITINGEKRIFATSQALVDELCDESRFHKAVMGGLEKLRMLASDGLFTAYHGERGWGIAHRILVPAFGPLRIRNMFEEMNDVAQQLCLKWARQGSSTSINITDDFTRLTLDTIALCTMNFRLNSFYNNETMHPFVKSMLYVLRESDIQSMLPGIANCIRVKARSRMSKHIQLMRNMARGIIQERRDQAEPVDDLLNTLLNGRDPVTGEGMSDDLIINNVITFLIAGHETTSGLLSFTFYYLLQNPHILERAQNEVDEVTGGERITVQHLGRLTYIDAILKESLRLMPSAPAFTVTPKKPEVLGGAWAIDAGQAVNVLLAVCLRDRSVFGPDADEFRPERMLEENFSKLPPNSWKPFGNGERSCIGRAFAWQEAQLVVAMVLQTFDLVPDDPSYKLRIKQTLTIKPDGFRVRATLRHGMTPTELEHVLAGNGATSSSTHNIKAAANSDTKAGGSGKPMAIFYGSNSGTCEALANRLASDAPGHGFSATTVGPLDQAKQNLPEDRPVVIVTASYEGQPPSNAAHFIKWMEDLNGNEMEKVSYAVFACGHHDWVETFHRIPKLVDSTLEKRGGTRLVPMGSADAATSDMFSDFEAWEDTVLWPGLKEKYKISDEESGGQKGLLVEVSTPRKTSLRQDVEEALVVAEKTLTKSGPAKKHIEIQLPSAMTYKAGDYLAILPLNPKSTVARVFRRFSLAWDSFLKIQSEGPTTLPTNVAISAFDVFSAYVELSQPATKRNILALAEATEDKATIQELERLAGDAYQAEISPKRVSVLDLLEKFPAVALPISSYLAMLPPMRVRQYSISSSPFADPSKLTLTYSLLDAPSLSGQGRHVGVATNFLSHLTAGDKLHVSVRASSEAFHLPSDAEKTPIICVAAGTGLAPFRGFIQERAAMLAAGRTLAPALLFFGCRNPEIDDLYAEEFERWEKMGAVDVRRAYSRATDKSEGCKYVQDRVYHDRADVFKVWDQGAKVFICGSREIGKAVEDVCVRLAIEKAQQNGRDVTEEMARAWFERSRNERFATDVFD

SEQ ID NO: 41 - protein, 617 aa, synthetic construct
RGGNATDFQSELQSLGTQAKGVRSDSAVSMSSAQADKGDKKPLTILYGSNSGTCEALAYRLASDAALHGFYARKIAPMNAACNDLPKSEPVIILAASYDGSPSDNAVEFFEWLNKAEPESLKGVSYTVFGCGHRDWVATFQRIPILIDDLLEKAGADRFADRGLADAAVMDLFVELEKWTTDVVWPAISQNEGGRDDDGETRLINSLLKLEVSQPRQLRQYSQLVQATVTESRSLTTEAALGKKMHLDMKLPPGVSYHPGDHLLVLPVNPPRDVKRVLSRFRLAWDTVVRASGEQYAHIPVETSMTAHELLSSYFELSQPATPRDIRVLAAAATDQKTKHLLSNLATASYAEGIHENRVSLLDLLEEFADIPLAFGTFIALLPSLRLRTYSISSSPSWEPNHASLTFSILDEPPTPPRTKSFLGVASNYLANLTPGDLIHVATRPVKNIFQMPADVSKVPIIMVAAGAGVAPFRGFIQERASQQQNGTELAPAALFFGCRSSDDDLYHHELDYFEDSGVVQVFRAYSREEDAGSQPSTRKGYVQESLLAEKQAFLRLWNAGAKIYVCGGVRMASGVKEAVMKLVYMAEQDATSKSFTPQEWFKRFEKARYAAEIFAA

SEQ ID NO: 42 - protein, 614 aa, synthetic construct
RGGNATDLQSELQSSGTKTKGVRTDSAVSITSAQADKKNKKPLTILFGSNSGTCEALAYRLASDAALHGFYARRIAPMNAACNNLPKSEPVIILAASYDGSPSDNAVEFFEWLNKVEPDSLKGVSYTVFGCGHRDWVATFQRIPILIDDLLEKAGADRFADRGLADAAVMDLFVELEKWTTDVVWPAISQTEGGGDGGESRLINSLLKVEVSQPRQLRQYSQLVQATVTETRSLTTESAIGKKMHLGIQLPPGVSYHPGDHLLVLPVNPPRDVKRVLSRFHLAWDTVVRASGDESVHIPVETSMTAHELLSSYFELSQPATPRDIRVLAAAATEQKTRHLLSSLATASYAEGIHENRVSLLDLLEEFPDIPLAFGTFIALLPSLRLRTYSISSSPSWEPNHASLTFSVLDEPPTPPRSKSFLGVASNYLANSTPGDLIHVATRPVKSIFQMPTDASKVPVIMVAAGAGLAPFMGFVQERASQQRNGTELAPAALFFGCRSSHDDLYHHELDCFEDSGAVQVFRAYSREDAGSKPNTRKGYIQDALLAEKQAFLRLWNAGAKIYVCGGVRMASGVKEAVMKLVYMAEQDAASKSFTPQEWFKRFEKTRYAAEIFT

SEQ ID NO: 43 - protein, 618 aa, synthetic construct
RGGTATDFQRELHSVGSTHDVRPSLTPSTNTLSQAGEGNKKPLTIFFGSNSGTCEALAYRLASDATLHGYYARTIAPLNAACGNLPKSEPVLILAASYDGLPSDNAEEFFDWLNKAEKDSLNGVSYAVFGCGHRDWVATFQRIPILIDDLLQRAGAERFAARGLADSAVMDLFVELEKWTADSVWPAITQTDGGGGERDDDGEGRLITSLLKVDFSQPRQLKQYSQLVEASVTESRTLTTASAIGKKVHLDIQLPSDQSYSPGDHLLVLPVNPMQTVKRVLSRFHLTWDTIVRASGNDSVRIPADTSMTAYELLSSYFELSQPATPRDIRVLAAAAEDEATKQLLTDLATSSYVEGIQEKRTSLLDLLERFPEIPMALGTYLTLLPSLRLRTYSISSSPSWNPGHATLTLSVLEEPATADCGSHFLGVASNHLAGLSRGDLIHVATRPVKGIFRMPTDLSKVPIIMIAAGSGLAPFRGFIQERAIQQRNGTELAPAALFFGCRSSDDDLYRSELDEFESAGVVRVFRAYSREDVGSRPNTRKGYVQDSMTAEKDAFAQLWNAGAKIYVCGSVRMASQVKDVVTKLVYAAETLGASKQFTPQEWFKRFEKTRYAAEIFT

SEQ ID NO: 44 - protein, 612 aa, synthetic construct
SGRTPRELCGFSNPISTNIQIKNGTIAPANDGQVGNSTPVTILYGSNSGTCEALAHRLARDAPSYGYSVTTVATLDSVIGILPRAKDELVVIITCSYDGLPADNAVRFCNWLKTLDEDALGGMPFAVFACGHHDWAKTFYKVPIMIDELLARAGAHRVAQMGKANSAVSDMFSDLENWEDEHLWSSSSAAGNETVDNVARTEVKQDITITNPRARALHHNAVECIVSETHKLSESGSALKYHVEIQLPANMKYAPGDHLSVLPINPRQNVRRALARFHLAGDSVLSVARIGHMGTLGQETLSAFDVFASYVELSQPATRRNIATLLSVTPEGEGRYELAELGGAAFESQIRDMRVSVLDLLERYKAIKLCVGAFIDMLPPLRVRTYSISSSALWKPSHASLTISVLAQPALSGQGSFLGVASNFLADLVPGDAVHFTIRPCKKQFHLPDDASAHPIIMVAAGSGIAPFRGFIQDRALQRRNGVQLQPAILFFGCRGSKQDDLYRQELDEFEAEGVVSVRRAFSGEETVSISESRMYVQDRMWADRAEVIQLWNLGAKIYVCGGINMADGVREIFNEIVGPTEVDGKRGAESLNKLSEETMSARYVAEIFSQT

SEQ ID NO: 45 - protein, 608 aa, synthetic construct
EHKTTAGMVPESVQSLRQAQKSGKPGNSKSSANESMVGQGNGRSVSIFYGSNSGSCESLANLLASDCAKYGFSVQTTDALDAARENFTSNQIVLIVASTYDGKPADNATEFVNWLKSLTGEPLKGVSYAVFGCGHHDWATTFYKIPILIDELLEQRGAQRVAPRGAANAAVSDLFSDLEKWEESILWPALGLTPALLESQGDTQPRLTISFQRPYTQRKEFLEATVTSASELTTSASPSRKCHMELQLPQDMTYNTGDYLAVLPLNPLSNVQRALSRFHLAWDSVLVIEATGPTQLPTATPISVADLFGAYVELSQPATPRNIRALAGAASDEPTKQALLKLAEDDFATQVRDKRLSVLDLLGQYESVALPVEAFIEMLLPLRPRTYSISSAPQWNPSHASITWSIIDTVSWSGHGRFLGVASNHLYDLSPGAVVRVSVRRSNPAFHPPQDPNSYPIIMIASGSGLAPFRGFIQERALQQKAGMTLAPALLFFGCRGRDDDLHRAEMDDFEKSGVVRVMRAYSKAPNEPDAFGCAYIQERVWSERKEFRELWNRGATVFVCGGTKMSEAIKDVFIKIAYGNAGQDDNKSSRDWFASLDPHRYVAEVFN

SEQ ID NO: 46 - protein, 606 aa, synthetic construct
NSTPPSKLVPKTAYSSSKADKSGDARSHPNMSIAGPGNNRSVSIFYGSNSGTCEALSNLLAGDFAKIGFTVQPIRTLDWAPHYFTTNELIVIVTATYDGRPTDDATEFVDWLESLQGEPLKGVSYTVFGCGNHDWATTLYKIPKFIDSTLEKHGAKRLAPLGTANVALRDPLSELEKWEEENLWPALDLALMPSNSPASLESGLKVSFQQPYTQRKEFTEATVTECYELTTSMSLTRKCHMELRLPEGVTYTAGDYLAVLPLNPMSNVQRALSRFHMAWDSVLVIESTGPTLLPTATPISVADLFSTYVELAQPATSRNLKMLASAATEQSSKQALLNLANDDSATTPSTKLPSVLDLLEAYPSLPLPIETFLEMLPPLRPRTYSISSSPSFHPSHATLTWSVVHAPSWSGHGSFYGVASNHLSDLSPGAVVRVSVRRSNPAFHPPTDPKAHPIIMIASGSGLAPFRGFIQERAWQKKAGMTLAPAMLFVGCRGKDDDLYRSAMDEFVREGVVRVFRAYSRMPEQVDAGGCMYVQERVWAERREFAELWSRGAKVFVCGGSQMSEAIKEVFANIGFAAEGEKSGGVSPDEWFRGLDPGRYATEVFN

SEQ ID NO: 47 - protein, 605 aa, synthetic construct
QSRAPLTFLDGSELSTKQAHTPQEGRKSIMKTPTVGQGKYKSVSIFYGSNSGTCEALSNVFANDCAKAGFLVQPIRALDSAKEDFTSNELVVIITATYDGRPTDNATEFVDWLNSLTGKPLEGVSYAVFGCGHQDWPATLYKIPNFIDEVLEQRGAKRIAPRGAVNVAISDPFSVFVNWEEETLWPAVGLSLTPSESPISLESQLKVSFQQPYTQRKEFKEATVTKLLELSSPASLTRKCHIELRLPQGMSYTTGDYLAVLPLNPASNVQRALARLNLAWDSVLIIESAGPTQLPTATPISVADLFGAYVELSSPASSRSIKRLAAAAIDESTKDSLLRLASNDSSQLGRKQYSVLDLLEDYESIPLSVEMFLEMLPPLRPRIYSISSAPDWKPSHTTLTWSVVNAPSWSGHGSFLGVASNHLYNLSPGAVVRVSVRRSNPAFRLPQDPGAYPIILIASGSGLAPFRAFLQQRALQHKTGIVLAPALLFFGCRSKDDDLYRTELDEFESSGIVRVRRAYSKTPKEIDACGCTYVQDRIWAEKEEFRTLWDQGAVVFVCGGSKMSEAVKDVFIKIARKSSSDNCLTSFTEWFKALDSRRYVAEIFN

SEQ ID NO: 48 - protein, 605 aa, synthetic construct
QSRTPHTFLNGSELTPKQTHTTQEGRKAIMKNSVLGQGKYKSVSIFYGSNSGTCEALSNVFANDCAKAGLVVQPIRALDCAKEDFTSNELVVIITATYDGRPTDNATEFVDWLTSLTGKPLGGVSYAVFGCGHQDWPATLYKIPNFIDEVLEQRGAKRIAPRGAVNVATSDPFSMFEEWEEETLWPALGLSLAPPESSTSLESGLKVSFQQPYTQRKEFKEATVTKHFELSSPASLTRKCHIELRLPQGMVYTTGDYLAVLPLNPTFNVQRALARFHLAWDSVLMIESAGPTQLPTAIPISAADLFGAYVELSSPASSRNIKRLAAAAIDESTKDSLLKLASKDSSRLGSQQRSVLDLLEEYGSIPLSVEMFLEMLPPLRPRTYSISSAPDWKPSHATLTWSVVNAPSWNGHGSFLGVASNHLYNLSPGAVVRVSVRHSNPAFRLPQDPGAYPIIMIASGSGLAPFRAFLQERALQKRAGKALAPALLFFGCRSKDDDLYRDELDEFESLGIVRVSRAYSKTPKETEARGCAYVQERIQAEEEEFRKLWDQGASIFVCGGSKMSEAVKDTFINIARKGAGNDGFRSPTEWFKALDTRRYVAEIFN

SEQ ID NO: 49 - protein, 605 aa, synthetic construct
QSRAPLAFLNGSELSPKQAHNTQEGRKSIIEKSILGQGKYKSVSIFYGSNSGTCEALSNMFANDCAKAGFIVQPIRALNSAKEDFTSNELVVIITATYDGRPTDNATEFVDWLNSLTGKPLEGVSYAVFGCGHQDWPATLYKIPIFVDEVLEQRGAKRIAPRGAVNVATSEPFSVFEDWEEETLWPALGLSLTPSESSTSLESGLKVSFQQPYTQRKGFKEATVTKHLELSSPASLTRKCHIELRLPQGMSYTTGDYLAVLPMNPTANVQRALARFHLAWDSVLIIESAGTTQLPTATPISVADLFGAYVELSSPASSRNIKRLAAAAIDEPTKHSLLKLANNESSRLRGKQQSVLDLLEDYESIPLSVEMFLEMLLPLRPRTYSISSAPDWKPSHATLTWSVLDAPSWSGHGSFLGVASNHLYNLSPGAVVRVSVQRSNPAFRLPQDPGAYPIIMIASGSGLAPFRAFLEERALKHRTGKALAPALLFFGCRSKDDDLYRNELDEFESSGIVRVRRAYSKTPKETDACGCTYVQDRIRAEKEEFRKLWEQGASIFVCGGSKMSEAVKDLFINIARKSAGNDGFRSSAEWFKALDSRRYVAEIFN

SEQ ID NO: 50 - protein, 609 aa, synthetic construct
EGWTARKVEQHLNRSSDLETSTAKAHPTPTAPTTQDSGHPITILYGSNSGTCEALAHTLAADARGHGFLPTTVDTLDSAKQNLPADQPIVIITASYEGQPTDNAGHFYNWLQNVTELKTSYAVFGCGHSDWRQSFHRIPASIDSLLEKAGSTRICKMGSADAAKGDMISDFNGWEDETLWPALQKQYTCTDSETSSPSHGQSLSVEVFSKRASHLRSDVSEARVVSAKVLSAPGVAPKRHIELQLPSDMTYRAGDYLAVLPLNPAETVHRVLARFSLPWDAMLSITSKDHTTLPTEHPVSAQDLFSAYLELSQPATKRNVTMLIEVSHEEAIKEELKKLSDDFDAEITAKRVSLLDLVERFPTIALPLSAFISSLISMRVRQYSISSSPLSDQNRATLTYAVLDEPSTSGQGRHIGVASNYLSQLKPGDIVHVAVKPSHASFHLPSSPETTPIIMIAAGAGLAPFRGYIQERASQIQSGRTLAPAHLYFGCRYPDKDDLYRDEIDSWEQIGAVVAHRCFSQAPEQSGGHKHVDGLILQDKELLSELWEGGAKVYVCGSRGLGDSVKKAFIKLAKEKAVADGNKNAEDDAKLEKWFESLRNERFATDVFT

SEQ ID NO: 51 - protein, 617 aa, synthetic construct
EGLTPSMLEARLAGSIVKTGGAKAKAQEAKDKTSGATAASRGRKLAVFYGSNAGTCEFMAQRLASDAQAHGFSATVDSLDAARESLPTDGPVVIVTSSYEGQPPHNAALFVDWLTNLKGNELKNVSYAVYGCGHADWVKTHQRVPKLVDSSLEERGAQRIIPLGTTDAKDRDMFSDFEAWEDEQLWPALTKQFGGQEKGDAENGVPDAGLSVSFSTPRTSTLRQDVRDALVLNARVLAPGKLGPVKRHIEVQLPTNMRYSAGDYMAILPHNPKDTVARVMRRFHLTWDSHVTIQASGPTTLPTDTSIPVSDVLSSYVELCQTATKRNISGLAQLAKDDKIRSQLQHLATDGYDSEIKAKRLSVLDIAERFVSLSIPFNHFLLMLPPMRVRQYSISSSPLVDPGVATLTYGVLDGPAISGQGRHIGVTSSYLASLTAGDKLQISIRAAQGGFKLPVDMDKTPLLCVAAGTGLAPFRAFFQERATLLANGRKLAPAILFFGCRDPAEDDLYRDEFDAWEAAGAVKVHRAYSRKTEASDGCRYVQDRIWREREALFDLWDRGARVYVCGSGQVAEGVKEVVMKAAVEKSEKDDGKAMTKEELEEWFNGIRNERYATDVFD

SEQ ID NO: 52 - protein, 614 aa, synthetic construct
DNLTPTTLERRLAGLAEPEKHAEQAKANGAAAAAAEGETGMPMTILYGSNSGTCEALAQRVASDAASHGFRATKIDCLDSANGSLPTDQPVVIITASYEGEPPDNAGHFVAWIEGQNKAKTPLKDVSYAVFGCGHKDWVQTFHRVPKLVDTTLAQLGATRLADLGLTDVSSGEVFTNFESWEDDVLWPSLANKYKTTSADDSKTKGVGVVISNPRTSTLRQDVKEATVTKTATLTKGSDPKSIKKHIEIKLPEGMTYSAGDYLAVLPINPKETVHRAMRRFHIARDAHLSIKTDGPTSLPVDTSVPANDLLSSYVELSQPATRRNLLALAEHAKDESTKVELNRLSGDAYADEISGKRASVLDLLERHPSIDLPIGVFLSMMPPMRVRQYSISSSPMAYPNNVTLTFSVLNEPSLSGQGPYIGVASSYLDSLAEGDSLHVSIRPSHAAFSLPSDAEHTPMVCVAAGTGLAPFRGFIQERATMLAAGRTLAPALLFFGCRSPEQDDLYRDEFDKWEEIGAVSVRRAYSRDSEKSEGCKHVQDRMWRDREELVSLWKKGAKGYVCGSRGVAEAAKETIIKIKVEMEKAKGEATDEESVKEWFEAQRNIRYVTDVFD

SEQ ID NO: 53 - protein, 611 aa, synthetic construct
DGLTPTSLSARLAGTPNTGQEDNSAQNGGVEESASGPGQPISVFYGSNSGTCETLARRIAADAPRHGFKVTAVDCMDVANGNIPKDQPVVFLTASYEGEPPDNARHFVSYLENLEDKKALEGVNYAVFGCGHHDWTSTFYRIPKLVDSALNENGATRLAESGFADAGGADVFVAFETWEDSTFWPALKEKYGIETANGANDEQLSLTVAVTNPRSEVLHTDVQLAQVIETRILTADGEPIKKHIEIKLPSDSSYRSGDYLAVLPINPRETVARAMRRFELPWDSHLEIEGGGHVDLPTEKSLPAFDIFGSYVELAQPATKRNILALAASTEDEADKAMLKDLADASYEKEILEKRMSVLDLLETYPSAKLSLGSFLAMLPRMRIRQYSISSSPLWKPNHVTLTFSLLNEPSKSGHGSYVGVATSYLASLQAGDKLPVSIKQSHASFHLPQDQGSTPIICVAAGTGLAPFRGFIQERAALIEAGRKVAPLVLYYGCREPGRDDLYADELAKWEKLGAVKVKHTYSRTPDKSDGNKYVQEAIWADRREVGELWENGAKVFVCGSKRLSTGVQDVAIRMTRAKGKLVGKEVSEEEAFKWWQELRNVRYATDVFE

SEQ ID NO: 54 - protein, 610 aa, synthetic construct
NGISATSLERALAYATIKDLPPATGAQQTRRGTIAKGKPIAIYYGSNTGTCEALANRLASDAANHGFRADVVDTLDSVRENLPTDKPIVIVTASYEGQPTDNAAHFVNWIGTLKEKELEKVTYAVFGCGHHDWAKTFHRIPKYLDATLEERGAIRLVGMGSADAAHGDIFTDFESWEDQVLWPTLRQKYGSSEAEGEGSLEATLDVEISTPRSTILRQDVREARVEEVAVLSASGVAEKRHIEISLPSDMSYSAGDYLAILPLNPKENIRRATKYFGLSWDSMLTISSAGPTTLPTDTPISAIDVFGAYIELAQPASKRNLLALADATRDDKTKNELTRLAEETFAEEITAKRVSVIDLLERFPSVQLPLGVFLKMQPPMRVRQYSISSSPLWNLNSVTLTYSVVNQPALSGQGRYVGVASNYLSNLVPGDKLHVSVRSSHQAFHLPKDAKNVPVIMMAAGTGLAPFRGFIQERAAQLAAGRSLAPALLFYGCRSPESDLMYPELLKRWEQLGAVTLRFAFSCGPQQSEGCKYVQDRVYYDRSEIVELFDAGAKLFVCGSREVGEGIQETLIKIAKERRKEVDGKEVDDTKAREWFEGLRNERFATDVFA

SEQ ID NO: 55 - protein, 608 aa, synthetic construct
NHMSATALERALASATISEPTGKTETGGAQKKAAQGKPLAIYYGSNSGTCEALANRLAGGAANHGFRVETIDILDSVKENLPADKPVAIVTASYEGQPADNAAHFVTWLDSLKASELNNVSYAVFGCGHRDWASTFLKVPKYIDAAIEERGGTRLVGMGSADASHGDIFTEFEAWEDKVFWPALKERYGTSDAVDGGPFEASLDVDVSTPRSSMLRQDVREAKVEAVEVLSAPGAPEKRHIEISLPSDMNYSAGDYLAILPLNPKENVARAMRYFGLAWDSVLTINSSGPTTLPVDTPCPASDVLGAYVELAQPATKRDIAALIEATPKEEEKKELTRLVEDVFATEITEKRVSVLDLLERFPSVQLPLGPFLKMLPPMRVRQYSISSSPLWNPDNVTLTYAVVDQPAFSGQGRFVGVASSYLSSLAPGDKLHVSVRSSHQAFHLPKDAKNVPVIMVAAGTGLAPFRGFIQERAAQLAAGRPLAPALLFYGCHSPDTDLLYSDLLKKWEGMGAVDIRPAYSRAADQSEDCKHVQDRLYHDRKDVVELWDKGAKVFVCGSREVGKGVEDACLKIAVEERRAKGKEGDEEKVGKWWEGLRNERFATDVFA

SEQ ID NO: 56 - protein, 606 aa, synthetic construct
DGMTPTELEQRLAGKGVVEEHHADKKAASAASSKGPQNPLAVYYGSNSGTCEALAQRVATDAASHGFKVTDLAPLDSANQNLPKDRPVIIVTASYEGLAPSNAAHFVSWIENLKGNELEGVSYAVFGCGHHDWVQTFHRIPKLVDSTLAEHGATRLIPLATTDAADRDMFSDFETWEDESLWPALKEKYGATAASTDGSAGGLIVDVSLPRKSTLRQDVEEALVTSTKTLTTSGSTKNHIEVRLPTGMTYRSGDYLAVLPFNPKDTVARVFRRFQLSWDAVLTIHSDQPTALPTDTPVSANDLLRAYVELSQPATKRNISVMAEATQDEELKAQIEKLAGDDYQQEISAKRVSVLDLLERFPSLEFPLDTYLAMLPPMRVRQYSISSSPLADPSKLTLTYSVLEQPALSGQGSHFGVATHFLASLAPGERLHVAVRQSKAFHLPTDAENTPLIFVGTGSGLAPFRGFVQERAAMIAAGRKLAPALLFYGCRSPDMDDLYAEEFERWEKLGAVEMRKAYSRASDKSFGCKYVQHRMSHDREDLFKLWDQGAKVLVCGSRDVGKAVEAVCVELVQEKAKADGKEVDEEKAQAWWDKQRNERYVTDVFD

SEQ ID NO: 57 - protein, 608 aa, synthetic construct
HGMTPTELEHVLAGNGATSSSTHNIKAAANSDTKAGGSGKPMAIFYGSNSGTCEALANRLASDAPGHGFSATTVGPLDQAKQNLPEDRPVVIVTASYEGQPPSNAAHFIKWMEDLNGNEMEKVSYAVFACGHHDWVETFHRIPKLVDSTLEKRGGTRLVPMGSADAATSDMFSDFEAWEDTVLWPGLKEKYKISDEESGGQKGLLVEVSTPRKTSLRQDVEEALVVAEKTLTKSGPAKKHIEIQLPSAMTYKAGDYLAILPLNPKSTVARVFRRFSLAWDSFLKIQSEGPTTLPTNVAISAFDVFSAYVELSQPATKRNILALAEATEDKATIQELERLAGDAYQAEISPKRVSVLDLLEKFPAVALPISSYLAMLPPMRVRQYSISSSPFADPSKLTLTYSLLDAPSLSGQGRHVGVATNFLSHLTAGDKLHVSVRASSEAFHLPSDAEKTPIICVAAGTGLAPFRGFIQERAAMLAAGRTLAPALLFFGCRNPEIDDLYAEEFERWEKMGAVDVRRAYSRATDKSEGCKYVQDRVYHDRADVFKVWDQGAKVFICGSREIGKAVEDVCVRLAIEKAQQNGRDVTEEMARAWFERSRNERFATDVFD

SEQ ID NO: 58 - protein, 622 aa, synthetic construct
HGMSPTELEHRLMGRGLNKTRLPSPLSAQTNGHQTNGSKESNVGGIPISIYYGSNSGTCEALAQRLATDATTHGFHATTVEPLDTARENLPKDRPMVIITASYEGQPPENAAHFVAWLESLKAQELSNVPYAVFGCGHHDWVQTFHRIPKLVNTIMEERGANRVAPMGLTNAADRDMFSDFEAWEDKVLWPALTEKYDVTEARKANGSDSGLVVQISYPRTLALRQDVEEAVVVEEKTLTAPGAAQKKHMDIQLPPGMTFRAGDYLAVLPLNPREHVERVFHRFQLARDTCLTIFGDKRTALPVNQTISAYDILSAYVELAQPATKRNVLSLLEATKDAETIEKLERLASAEYQAEITQKRVTILDLLERFPSITLPIGSFLAMLPPMRVRQYSISSSPLRNPSQATLTYSILGGPSLSGQGRYLGVATSYLASLKAGDKLHVAVRPSHVAFHLPHQPEKTPLILVAAGSGIAPFRGFIQERAAMIAAGRTVAPALLFYGCREPGIDDLYRQELDAWEGMGAVTVRRAYSRAAEQSEGCRYVQHRLYHDKKDVAGLWAKGAKLFVCGTRDVGKAVEEACVRVILESAKEGGGQPELRDLDYEGAKKWFEGIRNERYATDVFD

SEQ ID NO: 59 - protein, 617 aa, synthetic construct
HGLTPTQLEHRLNGTTAPTTPIENAASGLSLGGDQKANAAKGKPMAIYYGSNSGTCEALARRLATDAPSHGFNATVVDSLDAAHQNIPTDKPVVVVTASYEGEPPDNAALFCNWLQALKDKELENVNYAVFGCGHHDWSQTFHKIPKMVDATIEKHGGTRIAALGVADAAEGDMFTKFETWEDELLWPALREKYGVEDSVQDGESLQGGLSVEISTPRSSTLRQDVKEAVVIETRDLTSPEAPTKRHIEIQLPSDMTYTAGDYLAILPINPKAVVHRALRRFRLAWDAHVTINSNSTTTLPTGYPVPASDVLGAYVELGQPATKRGILALAEATKDEDAKAALQRLAGDDYVTEVNAKRRSVLDLLEQFPAVELPFATFLALLPPMRVRQYSISSSPLWHQNHLTLTYAVLDAPALSGTGQRYLGVATSYLSSLAEGDKLHVAIRPSHASFHLPVDTEKTPIMCIAAGSGLAPFRGFIQERAAMLDAGRTLAPALLFFGCRHPDQDDLYREELDRWQAMGAVDVRRAYSRDPASSEGCAHVQDRLWNDRAEVQALWDRGAKVFVCGSREVGDAVREVAIRMYVDGAKVNHGRDLSVEEATEKFDAMRNERYASDVFD

SEQ ID NO: 60 - protein, 612 aa, synthetic construct
NHMTATTLERALASTTISAAEAAKSKGQTVSKGPKAQGKPMSILYGSNTGTCEALANRLASDAANHGFHAQVVDTLDSAKEKLPTDRPVVIVTASYEGNPADNAAHFVNWIGGLKSNELNDVSYAVFGCGHRDWVKTFHRIPKYLDSVIEKRGGTRIAPIGTTDAAAGDIFTDFETWEDQVFWPAMREKYGTAEGSTDGSLETSMDVEISSPRSSTLRQDVREARVEEVRTLSAPGSPEKRHIEISLPSDMTYSAGDYLAILPINPKESIARAMRYFGLPWDSILTLSASAGPTTLPTGQPIAAADMFGAYVELAQPASKRNILALIEASTDEKTKEALTNLSDSAFTTEITQKRISVLDLLERFPSVQLPLGAFLKMMPPMRVRQYSISSSPLWNSNSVTLTYSVVDQPAMSGQGRFVGVASNYLSSLAPGDKLHVSVRQSHQSFHLPKDSKNVPVIMIAAGSGLAPFRGFIQERAAQIGAGRPLAPALLFYGCHSSDTDSLYADEIARWEKMGAVSARYTFSRAPEQSEGCKYVQDRLYHDRKDVVELFDRGAKLFVCGSRAVGEGVQNTCVRIVKEQSEEVHGCDVSEKKARAWFDGLRNERFATDVFA

SEQ ID NO: 61 - protein, 612 aa, synthetic construct
NHMTPTTLERALSSTSIGESAAANTGKPKPQTANNSVTQGQPIRIYYGSNTGTCEALAQRLASDASAHGFTADLVDTLDSAKESLPTDKPVIIVTASYEGQPTDNAAHYVNWLGGLKGSELEKVPFAVFGCGHRDWAKTFLKIPKFVDEIMEQRGGTRLVPMGSADAAGGDIFTEFETWEDQVLWPALREKYSSADTSGGSSYDTSLQVEISTPRSSMLRQDVKEARVEEVEILSKPGVPEKRHIEISLPSDMTYSAGDYLAILPINPKNNVTRAMRHFGLAWDSVLTISSVGPTTLPTNAPISAADVFGAYVELGQPASKRNLQALIEATEDEGAKKDLTELSDISFATEITGKRVSVLDLLERFPSVELPLGHFLKMLPPMRVRQYSISSSPLWNPNHVTLTYAVVDQPALSGQGRFVGVASSYLSSLAAGDKLHVSVRTSHQAFHLPKDTKNVPVIMIAAGTGLAPFRGFIQERAAQIAAGRSLAPALLFYGCRSPDSDALYSSLLQRWETLSAVSVRYSYSRAADQSEGCKYVQDRLYHDRKEVVELFEKGAKIFICGGREVGEGVQNICMKIMREETESMGKDVSEQRAKEWFEGLRNERFATDVFA

SEQ ID NO: 62 - protein, 613 aa, synthetic construct
TGMSATTLERALGSASINAPSAGTQSAKSHAPTKTSGKKGKPLAIYYGSNTGTCEALAQRLAGDAANHGFSAETVDILDTAKEKLPTDKPVVIITASYEGQPADNAAHFVKWLEALEGSELENVSYSVFGCGHHDWSTTFHKIPKLIDNGLEKRGAKRIAPIGTADAATGDIFSDFETWEDRVFWPAVSEQYGVEADDSASTYESSINVEVSSPRSSTLRQDVREARVEETKVLTAPGAPEKRHIEISLPSDMTYNAGDYLAILPLNPKENVNRAMRYFGLAWDSMLTIQSTGPSTLPMDAPIPASDLFGAYIELAQPASRRNINSLIEATQDEETKAELTRLAGDAFPEEIISKRVSVLDLLERFPSIQLPLGPFLQMQPPMRVRQYSISSSPLWNPSHVTLTYAVLDQPAYSGHGRFVGVASNYLSTLSPGDVLHVSVRSSHKAFHLPKDGQNVPIICIAAGSGIAPFRGFIQERAAQLAAGRTLAPALLFFGCHSPTTDSLYSAELSRWESLGAVSVRHAFSRAPDQSLGCKYVQDRLWHDRKEVVELFERGAKIFTCGSKKVGEGVAETCIRIAKESVKESEGREADDRKAREWFEGLRNERFATDVFA

SEQ ID NO: 63 - protein, 603 aa, synthetic construct
HGMTPTELEQHLAGKLSSEHYHAMSRKTSSNISASAGKPLAVFYGSNSGTCEALAQRIAADAPRHGFRASTVAPLDDANQKIPKDRPVVIVTASYEGQPPSNAALFVAWMESLKGKEMENVSYAVFGCGHRDWVQTFHRIPKLVDSKLGELGGQRIVPLATTDAAERDMFSDFEAWEDESLWPALQKMYGTEDSGDSNGDGINVEVSLPRKSTLRQDVEEALVVSSKTLTDSGSVKKHIEIQLPTGMTYRAGDYLAVLPFNPKKTVSRVFRRFKLSWDATLKISSDRPTTLPTDTAISAADVLAAYVELSQPATKRNIQALADATQDQAVVQKLQKLAGEDYHENITVKRISILDLLEQFPTIALPFGSYLGMLPPMRVRQYSISSSPLADPAKVTLTYSVLEQPALSGQGAHVGVATNFLAGLVPGERLHVAVRPSKSFHLPSDAENTPIVCVAAGSGMAPFRGFMQERAAMIGAGRKVAPALLFFGCRNPDKDDLYADELARWEELGAVEVRRAYSRVPEKSFGCKYVQHRLSHDREEVYKLWDRGAKIYVCGSRDVGKAVEAVCIDMVQESGSSKDVTEDKAKAWFEKQRNERYVTDVFD

SEQ ID NO: 64 - protein, 626 aa, synthetic construct
DGLNPTTLERRLQGLGQGDALSTAPANNGAGPGSADATGAPLTVLYGSNSGTCEALARRIATDAPAHGFHAATVDCMDSAKGKLPTDQAVVIITASYEGQPPDNAGHFVAWLEGSDKTTRPFKDVSYAVFGCGHKDWAQTFHRVPRLVDDSLQELGASRLVEPGLSDVSTGAVFSDFESWEDEKLWPALASRYQVSSKASAAENGAEGSAVKVHVSNPRISTLRQDVKEARVIEAKALTSGPESTTDGGRQFKRHLEIELPEGMEYSAGDYLAVLPVNPIDTVLRTLHRFGLARDAHVTIEMSSSQSTALPTNTSVPAFELLSSYVELSQPATKRNLSALQDAASDAETKSQLDKLSGDDFVDEVGAKRVSVLDLLERFPTIDLSIGSFLSMMPPMRVRQYSISSSPLVNPSRATLSFSVLDAPSLSGQGRHIGVASNYLSSLTAGDSLQVSVRPSHAAFSLPSDPERTPLICAAAGTGLAPFRGFAQERAAMISAGRKLAPALLYFGCRAPDLDDLYRDEFDAWERLGAVEVRRTYSRRPEASDGCRHVQDRILADADRLLGLWRDGAKLYVCGSRGVSQSVMDAMTRIKMDKDRELHGKETTAEEARQYFDDLRNVRYATDVFD

(19) United States
(12) Patent Application Publication
(10) Pub. No.: US 2026/0193683 A1
(43) Pub. Date: Jul. 9, 2026

(54) ENZYMES, CELLS, AND METHODS FOR PRODUCING LACTONES

(71) Applicant: Manus Bio Inc., Waltham, MA (US)

(72) Inventors: Arthur J. SHAW, Waltham, MA (US); Stephen SARRIA, Waltham, MA (US); Liwei LI, Waltham, MA (US); Christine Nicole S. SANTOS, Waltham, MA (US); Ajikumar PARAYIL KUMARAN, Waltham, MA (US)

(21) Appl. No.: 18/722,982

(22) PCT Filed: Dec. 22, 2022
(86) PCT No.: PCT/US2022/053772

§ 371 Date: Jun. 21, 2024

Related U.S. Application Data
(60) Provisional application No. 63/293,357, filed on Dec. 23, 2021.

(51) Int. Cl. C12P 17/06; C12N 9/02; C12N 15/52; C12N 15/81

(52) U.S. Cl. CPC C12P 17/06; C12N 9/0071; C12N 15/52; C12N 15/815; C12Y 114/14001; C12N 2800/102

(57) ABSTRACT

The present disclosure relates to microbial hosts, enzymes, and methods for biosynthesis of lactones.

### CROSS-REFERENCE TO RELATED APPLICATIONS

This application claims priority to, and the benefit of, U.S. Provisional Application No. 63/293,357 filed Dec. 23, 2021, the disclosure of which is hereby incorporated by reference in its entirety.

### DESCRIPTION OF THE TEXT FILE SUBMITTED ELECTRONICALLY

The instant application contains a sequence listing, which has been submitted in XML format via EFS-Web. The contents of the XML copy named “MAN-034US 107590-5033 Sequence Listing,” which was created on Jan. 28, 2025 and is 111,983 bytes in size, the contents of which are incorporated herein by reference in their entirety.

### BACKGROUND

Lactones are cyclic carboxylic esters. Lactones are formed by intramolecular esterification of the corresponding hydroxycarboxylic acids, which takes place spontaneously when the ring that is formed is five-membered (γ-lactones) or six-membered (δ-lactones). Naturally occurring lactones are mainly saturated and unsaturated γ- and δ-lactones. The γ- and δ-lactones are intramolecular esters of the corresponding hydroxy fatty acids, and they contribute to the aroma of fruits, butter, cheese, and other foods.

For example, delta-dodecalactone (δ-dodecalactone) has fruity, creamy/buttery or milk odor notes, and is widely used as a flavor or fragrance. δ-dodecalactone also has anti-fungal properties. Yang et al., Purification and characterization of antifungal δ-dodecalactone from Lactobacillus plantarum AF1 isolated from kimchi, Food Chem. 368:130736 (2021). δ-dodecalactone naturally occurs in trace amounts in peaches and milk products. It is extracted from natural sources or chemically synthesized.

Therefore, sustainable and scalable processes for synthesizing δ-dodecalactone, as well as other valuable lactones, are needed.

### SUMMARY OF THE DISCLOSURE

The present invention, in various aspects and embodiments, provides enzymes (including engineered enzymes), engineered microbial strains, and methods for making valuable lactones using recombinant microbial processes. In other aspects, the invention provides methods for making products, including foods, beverages, and fragrances (among others), by incorporating the lactones produced according to the present disclosure.

In various aspects, the present disclosure provides microbial cells for producing one or more lactones. In some embodiments, the microbial cell expresses an enzyme having fatty acid hydroxylase activity. In some embodiments, the microbial cell is engineered relative to a parent strain. In some embodiments, the microbial cell is engineered relative to a parent strain to have a decrease in expression and/or activity of one or more enzymes involved in fatty acid activation and degradation. Additionally or alternatively, the microbial cell is engineered relative to a parent strain to have a decrease in expression and/or activity of one or more β-oxidation and/or peroxisome enzymes. Additionally or alternatively, the microbial cell is engineered relative to a parent strain to have a modification in expression and/or activity of one or more lipases. Additionally or alternatively, the microbial cell is engineered relative to a parent strain to have a decrease in expression and/or activity of ω-oxidation enzymes.

In some embodiments, the microbial cell is engineered relative to a parent strain to have a decrease in expression and/or activity of one or more acyl-CoA synthetase enzymes. In some embodiments, the microbial cell is engineered relative to a parent strain to have an increase in metabolic NADPH supply. In some embodiments, the microbial cell is engineered relative to a parent strain to have a decrease in expression and/or activity of one or more neutral lipid biosynthesis enzymes. In some embodiments, the microbial cell is engineered relative to a parent strain to have a decrease in expression and/or activity of one or more of citric acid cytoplasmic exporter, and one or more NADPH dependent aldehyde reductases.

In some embodiments, the enzyme having fatty acid hydroxylase activity expressed by the microbial cell is a cytochrome P450 enzyme selected from CYP505, CYP116, CYP703, and CYP102, or a derivative thereof. In some embodiments, the P450 enzyme has a ω-7 hydroxylase activity on C12 fatty acid substrate. In some embodiments, the P450 enzyme comprises an amino acid sequence that is at least 50% identical to an amino acid sequence selected from SEQ ID NOs: 1, 3, 4, 5, 8, and 11. In some embodiments, the P450 enzyme comprises an amino acid sequence that is at least 60% identical, or at least 70% identical, or at least 80% identical, or at least 90% identical, or at least 95% identical, or at least 97% identical, or at least 98% identical, or at least 99% identical to an amino acid sequence selected from SEQ ID NOs: 1, 3, 4, 5, 8, and 11. In some embodiments, the P450 enzyme comprises an amino acid sequence that is at least 50% identical, or at least 60% identical, or at least 70% identical, or at least 80% identical, or at least 90% identical, or at least 95% identical, or at least 97% identical, or at least 98% identical, or at least 99% identical to the amino acid sequence selected of SEQ ID NO: 1.

In some embodiments, the P450 enzyme comprises an amino acid sequence that has at least 80%, or at least 90%, or at least 95%, or at least 97%, or at least 98%, or 100% sequence identity to amino acids 1 to 461 of SEQ ID NO: 1. In some embodiments, the P450 enzyme comprises a CPR domain having at least 70% sequence identity, or at least 80% sequence identity, or at least 90% sequence identity, or at least 95% sequence identity, or at least 97% sequence identity, or at least 98% sequence identity or 100% sequence identity to a CPR domain from selected from SEQ ID NOS: 37 to 60.

In some embodiments, the P450 enzyme comprises an amino acid sequence that is at least 70%, or at least 75%, or at least 80%, or at least 85%, or at least 90%, or at least 95%, or at least 98%, or at least 99% identical to an amino acid sequence selected from SEQ ID NOs: 17 to 36. In some embodiments, the P450 enzyme comprises an amino acid sequence that is at least 70%, or at least 75%, or at least 80%, or at least 85%, or at least 90%, or at least 95%, or at least 98%, or at least 99% identical to an amino acid sequence selected from SEQ ID NOs: 29, 33, and 34.

In some embodiments, the P450 enzyme comprises an amino acid sequence that is at least 80%, or at least 85%, at least 90%, or at least 95%, or at least 97%, or at least 98%, or at least 99% identical to the amino acid sequence of SEQ ID NO: 29, and comprising one or more mutations or one or more sets of mutations that increase the desired fatty acid hydroxylase activity and/or stereoselectivity. In some embodiments, such mutations or sets of mutations include one or more (e.g., 2, 3, 4, 5, or more) listed in Tables 3, 4, 5, and 6. In some embodiments, the P450 enzyme comprises a substitution of E443 with respect to SEQ ID NO: 29. In some embodiments, the P450 enzyme comprises a substitution of E443 with respect to SEQ ID NO: 29 with Gln or Asn. In some embodiments, the P450 enzyme comprises a substitution of Q53 with respect to SEQ ID NO: 29. In some embodiments, the P450 enzyme comprises a substitution of Q53 with respect to SEQ ID NO: 29 with an amino acid selected from Arg, His, and Lys. In some embodiments, the P450 enzyme comprises a substitution of P363 with respect to SEQ ID NO: 29. In some embodiments, the P450 enzyme comprises a substitution of P363 with respect to SEQ ID NO: 29 with an amino acid selected from Ala, Leu, Ile, and Val. In some embodiments, the P450 enzyme comprises a substitution of T332 with respect to SEQ ID NO: 29. In some embodiments, the P450 enzyme comprises a substitution of T332 with respect to SEQ ID NO: 29 with an amino acid selected from Ala, Leu, Ile, and Val.

In some embodiments, the P450 enzyme comprises a E443Q substitution with respect to SEQ ID NO: 29. In some embodiments, the P450 enzyme comprises a Q53R substitution with respect to SEQ ID NO: 29. In some embodiments, the P450 enzyme comprises a P363A substitution with respect to SEQ ID NO: 29. In some embodiments, the P450 enzyme comprises a T332S substitution with respect to SEQ ID NO: 29. In some embodiments, the P450 enzyme comprises a E443Q and/or Q53R substitution with respect to SEQ ID NO: 29. In some embodiments, the P450 enzyme comprises E443Q and Q53R substitutions with respect to SEQ ID NO: 29. In some embodiments, the P450 enzyme comprises one or more substitutions selected from E443Q, P363A, and Q53R with respect to SEQ ID NO: 29. In some embodiments, the P450 enzyme comprises E443Q, P363A, T332S and Q53R substitutions with respect to SEQ ID NO: 29. In some embodiments, the P450 enzyme comprises one or more substitutions selected from E443Q, T332, P363A, and Q53R with respect to SEQ ID NO: 29. In some embodiments, the P450 enzyme comprises E443Q, T332S, P363A, and Q53R substitutions with respect to SEQ ID NO: 29.

In some embodiments, the microbial cell is engineered relative to a parent strain to limit fatty acid activation and degradation. In some embodiments, the modifications comprise a reduction in the amount or activity of a long chain fatty acyl-CoA synthetase. In some embodiments, the modification that reduces the amount or activity of a long chain fatty acyl-CoA synthetase encoded by FAA1 gene, or a homolog, or an ortholog thereof. In some embodiments, the microbial cell comprises a partial or complete deletion of FAA1 gene, or a homolog, or an ortholog thereof.

In some embodiments, the microbial cell is engineered relative to a parent strain to cause a reduction in the amount or activity of a very long chain fatty acyl-CoA synthetase and fatty acid transporter. In some embodiments, the modification that reduces the amount or activity of a very long chain fatty acyl-CoA synthetase and fatty acid transporter encoded by FAT1 gene, or a homolog, or an ortholog thereof. In some embodiments, the microbial cell comprises a partial or complete deletion of FAT1 gene, or a homolog, or an ortholog thereof.

In some embodiments, the microbial cell is engineered relative to a parent strain to cause a reduction in the amount or activity of one or more fatty acyl-CoA synthetases. In some embodiments, the modification that reduces the amount or activity of one or more fatty acyl-CoA synthetases encoded by FAA2, FAA3, and FAA4 genes, or a homolog, or an ortholog thereof. In some embodiments, the microbial cell comprises a partial or complete deletion of FAA2, FAA3, and/or FAA4 genes, or a homolog, or an ortholog thereof.

In some embodiments, the microbial cell is engineered relative to a parent strain to cause a downregulation of β-oxidation and peroxisome metabolism. In some embodiments, the downregulation of β-oxidation and peroxisome metabolism is caused by a reduction in the amount or activity of a multifunctional β-oxidation enzyme. In some embodiments, the microbial cell comprises a modification that reduces the amount or activity of a multifunctional β-oxidation enzyme encoded by MFE1 gene, or a homolog, or an ortholog thereof. In some embodiments, the microbial cell comprises a partial or complete deletion of MFE1 gene, or a homolog, or an ortholog thereof.

In some embodiments, the microbial cell is engineered relative to a parent strain to cause a reduction in the amount or activity of one or more peroxisomal acyl-CoA oxidase. In some embodiments, the microbial cell comprises a modification that reduces the amount or activity of a peroxisomal acyl-CoA oxidase enzymes encoded by ANT1 gene, or a homolog, or an ortholog thereof. In some embodiments, the microbial cell comprises a partial or complete deletion of ANT1 gene, or a homolog, or an ortholog thereof. In some embodiments, the microbial cell comprises a modification that reduces the amount or activity of one or more peroxisomal acyl-CoA oxidase enzymes encoded by one or more of POX1, POX2, POX3, POX4, POX5, and/or POX6 genes, or homologs, or orthologs thereof. In some embodiments, the microbial cell comprises a partial or complete deletion of one or more of POX1, POX2, POX3, POX4, POX5, and/or POX6 genes, or homologs, or orthologs thereof.

In some embodiments, the microbial cell is engineered relative to a parent strain to cause a decreased expression or activity of lipases encoded by LIP2, LIP7, and/or LIP8 genes. In some embodiments, the microbial cell comprises a modification that reduces the amount or activity of the lipase encoded by LIP2 gene, or a homolog, or an ortholog thereof. In some embodiments, the microbial cell comprises a partial or complete deletion of LIP2 gene, or a homolog, or an ortholog thereof.

In various aspects, the present disclosure provides a microbial cell for producing one or more lactones. In some embodiments, the microbial cell expresses a P450 enzyme having fatty acid hydroxylase activity. In some embodiments, the P450 enzyme comprises an amino acid sequence that has at least 80%, or at least 90%, or at least 95%, or at least 97%, or at least 98%, or 100% sequence identity to amino acids 1 to 461 of SEQ ID NO: 1. In some embodiments, the P450 enzyme comprises a CPR domain having at least 70% sequence identity, or at least 80% sequence identity, or at least 90% sequence identity, or at least 95% sequence identity, or at least 97% sequence identity, or at least 98% sequence identity or 100% sequence identity to a CPR domain selected from SEQ ID NOS: 37 to 60.

In some embodiments, the P450 enzyme comprises a CPR domain having at least 70%, or at least 75%, or at least 80%, or at least 85%, or at least 90%, or at least 95%, or at least 98% sequence identity to an amino acid sequence selected from SEQ ID NOs: 53, 57, and 58. In some embodiments, the P450 enzyme comprises an amino acid sequence that is at least 70%, or at least 75%, or at least 80%, or at least 85%, or at least 90%, or at least 95%, or at least 98%, or at least 99% identical to an amino acid sequence selected from SEQ ID NOs: 17 to 36. In some embodiments, the P450 enzyme comprises an amino acid sequence that is at least 70%, or at least 75%, or at least 80%, or at least 85%, or at least 90%, or at least 95%, or at least 98%, or at least 99% identical to an amino acid sequence selected from SEQ ID NOs: 29, 33, and 34.

In various aspects, the present disclosure provides a microbial cell for producing one or more lactones. In some embodiments, the microbial cell expresses a P450 enzyme having fatty acid hydroxylase activity. In some embodiments, the P450 enzyme comprises an amino acid sequence that has at least 80%, or at least 90%, or at least 95%, or at least 97%, or at least 98%, or 100% sequence identity to amino acids 1 to 461 of SEQ ID NO: 1. In some embodiments, the P450 enzyme comprises a CPR domain having at least 70% sequence identity, or at least 80% sequence identity, or at least 90% sequence identity, or at least 95% sequence identity, or at least 97% sequence identity, or at least 98% sequence identity or 100% sequence identity to a CPR domain selected from SEQ ID NOS: 61 to 64.

In some embodiments, the P450 enzyme comprises a CPR domain having at least 70%, or at least 75%, or at least 80%, or at least 85%, or at least 90%, or at least 95%, or at least 98% sequence identity to an amino acid sequence selected from SEQ ID NOs: 53, 57, and 58. In some embodiments, the P450 enzyme comprises an amino acid sequence that is at least 70%, or at least 75%, or at least 80%, or at least 85%, or at least 90%, or at least 95%, or at least 98%, or at least 99% identical to an amino acid sequence selected from SEQ ID NOs: 17 to 36. In some embodiments, the P450 enzyme comprises an amino acid sequence that is at least 70%, or at least 75%, or at least 80%, or at least 85%, or at least 90%, or at least 95%, or at least 98%, or at least 99% identical to an amino acid sequence selected from SEQ ID NOs: 61, 62, 63 and 64.

In various aspects, the present disclosure provides a method for making one or more lactones, comprising: culturing a microbial cell of any of embodiments disclosed herein in the presence of a fatty acid substrate or an ester or glyceride thereof, and recovering the one or more lactones from the culture. In some embodiments, the fatty acid substrate or ester or glyceride thereof is added to the culture. In some embodiments, the fatty acid substrate or ester or glyceride thereof is synthesized by the microbial cell.

In some embodiments, the lactone is δ-dodecalactone (C₁₂H₂₂O₂), and the substrate is dodecanoic acid or an alkyl ester thereof. In some embodiments, is the substrate is a methyl ester dodecanoic acid. In some embodiments, the δ-dodecalactone comprises at least 50%, or at least 60%, or at least 75%, or at least 80% or at least 90% R-δ-dodecalactone. δ-dodecalactone can be recovered from the microbial culture. For example, δ-dodecalactone may be recovered from microbial cells, or in some embodiments, is predominately available in the extracellular media, where they may be recovered or sequestered.

In various aspects, the present disclosure provides a method for making a product, comprising, incorporating one or more lactones made according to the method of any of the embodiments disclosed herein into said product. In some embodiments, the product is selected from a perfume, a cosmetic, a food product, a beverage, a flavor, a food additive, a fragrance, a detergent fragrance, a green solvent, an antimicrobial ingredient, a polymer, a nylon precursor, and a fuel precursor.

In various aspects, the present disclosure provides a P450 enzyme having fatty acid hydroxylase activity, the P450 enzyme comprising an amino acid sequence that has at least 80% sequence identity to amino acids 1 to 461 of SEQ ID NO: 1, and comprises a CPR domain having at least 70% sequence identity to a CPR domain selected from SEQ ID NOS: 37 to 60. In some embodiments, the P450 enzyme comprises an amino acid sequence that has at least 90% sequence identity to amino acids 1 to 461 of SEQ ID NO: 1. In some embodiments, the P450 enzyme comprises an amino acid sequence that has at least 95% sequence identity, or at least 97% sequence identity, or at least 98% sequence identity to amino acids 1 to 461 to SEQ ID NO: 1. In some embodiments, the P450 enzyme comprises a CPR domain that has at least 80%, or at least 85%, or at least 90%, or at least 95%, or at least 97%, or at least 98% sequence identity to a CPR domain selected from SEQ ID NOS: 37 to 60. In some embodiments, the P450 enzyme comprises a CPR domain that has at least 80%, or at least 85%, or at least 90%, or at least 95%, or at least 97%, or at least 98% sequence identity to SEQ ID NO: 53, SEQ ID NO: 57, or SEQ ID NO: 58. In some embodiments, the P450 enzyme comprises an amino acid sequence that has at least 70% sequence identity, or at least 80% sequence identity, or at least 90% sequence identity, or at least 95% sequence identity, or at least 97% sequence identity, or at least 98% sequence identity to any of SEQ ID NOS: 17 to 36. In some embodiments, the P450 enzyme comprises an amino acid sequence that has at least 70% sequence identity, or at least 80% sequence identity, or at least 90% sequence identity, or at least 95% sequence identity, or at least 97% sequence identity, or at least 98% sequence identity to any of SEQ ID NOS: 29, 33, 34, 61, 62, 63 and 64.

In various aspects, the present disclosure provides a P450 enzyme having fatty acid hydroxylase activity. In some embodiments, the P450 enzyme comprises an amino acid sequence that has at least 80% sequence identity to amino acids 1 to 461 of SEQ ID NO: 1. In some embodiments, the P450 enzyme comprises a CPR domain having at least 70% sequence identity to a CPR domain selected from SEQ ID NOs: 61 to 64. In some embodiments, the P450 enzyme comprises a substitution at one or more positions selected from Q53, T332, P363 and E443 with respect to SEQ ID NO: 29. In some embodiments, Q53 is substituted with an amino acid selected from Arg, Ile, Leu, His, Lys, Gly, Ala, and Val. In some embodiments, T332 is substituted with Ser, Gly, Ala, Pro, Cys, Ile, Leu, and Val. In some embodiments, P363 is substituted with an amino acid selected from Ala, Leu, Ile, and Val. In some embodiments, E443 is substituted with Gln or Asn. In some embodiments, the P450 enzyme comprises one or more substitutions selected from E443Q, T332, P363A, and Q53R with respect to SEQ ID NO: 29. In some embodiments, the P450 enzyme comprises E443Q, T332S, P363A, and Q53R substitutions with respect to SEQ ID NO: 29.

In various aspects, the present disclosure provides a polynucleotide encoding the enzyme of any of the embodiments disclosed herein. In some embodiments, the polynucleotide further comprises a promoter.

In various aspects, the present disclosure provides a host cell expressing the polynucleotide of any of the embodiments disclosed herein. In some embodiments, the cell is a bacterium or yeast.

Other aspects and embodiments of the invention will be apparent from the following detailed disclosure.

### BRIEF DESCRIPTION OF THE DRAWINGS

FIG. 1 illustrates a biosynthetic scheme for δ-dodecalactone production using CYP505E3.

FIG. 2A and FIG. 2B show the effect of the expression in Yarrowia lipolytica of a cytochrome P450 CYP505-1 (SEQ ID NO: 1) having ω-7 hydroxylase activity. FIG. 2A shows the GC-MS profile of an isooctane extract of whole cultures (cells and broth). FIG. 2B shows magnification of the area of the GC profile indicated in FIG. 2A, and shows detectable production of other lactone products.

FIG. 3A and FIG. 3B show the activity of CYP enzymes for the biosynthesis of δ-dodecalactone. Yarrowia lipolytica strains expressing one of the CYP enzymes of SEQ ID NOs: 1-16 were generated and screened for biosynthesis of δ-dodecalactone. FIG. 3A shows the relative δ-dodecalactone titer produced by the Yarrowia lipolytica strain expressing the indicated CYP enzyme. FIG. 3B shows the fractions of R-δ-dodecalactone and S-δ-dodecalactone produced by the Yarrowia lipolytica strains expressing the indicated CYP enzyme.

FIG. 4 demonstrates that the deletion of FAA1Δ strain produces greater amounts of δ-dodecalactone compared to a wild type strain.

FIG. 5 demonstrates the production of δ-dodecalactone by CYP505E3 chimeras having a CPR domain swap.

FIG. 6 demonstrates the effect of various deletions of genes encoding lipases, and/or genes involved in the β-oxidation, and peroxisome metabolism, fatty acid activation and degradation, and ω-oxidation on the production of C12 free fatty acid species.

### DETAILED DESCRIPTION

In various aspects, the present disclosure provides methods for making one or more lactones, and provides enzymes and host cells for use in these methods. The present disclosure provides engineered host cells for producing lactone products by microbial fermentation or bioconversion. The invention further provides methods of making products containing lactones, including fragrances, cosmetics, food products, beverages, flavors, food additives, among others. Such lactone-containing products can be made at reduced cost and more sustainable fashion by virtue of this disclosure.

Accordingly, in one aspect, the present disclosure relates to a microbial cell for producing one or more lactones, including γ and δ lactones. In various embodiments, the microbial cell recombinantly expresses an enzyme having fatty acid hydroxylase activity, such that expression of the enzyme in the presence of the fatty acid substrate (e.g., either added exogenously to the culture and/or produced by the cell) will lead to production of the desired lactone(s). In various embodiments, the microbial cell is engineered relative to a parent strain to have one or more metabolic or genetic modifications to improve lactone production or yield. Such modifications can be independently selected from: (a) a decrease in expression and/or activity of one or more acyl-CoA synthetase enzymes; (b) an increase in metabolic NADPH supply; (c) a decrease in expression and/or activity of one or more neutral lipid biosynthesis enzymes; (d) a decrease in expression and/or activity of one or more β-oxidation and/or peroxisome enzymes; (e) a modification in expression and/or activity of one or more lipases; (f) a decrease in expression and/or activity of ω-oxidation enzymes; and (g) a decrease in expression and/or activity of one or more of citric acid cytoplasmic exporter, and one or more NADPH dependent aldehyde reductases.

In various aspects, the present disclosure provides microbial cells for producing one or more lactones. In some embodiments, the microbial cell expresses an enzyme having fatty acid hydroxylase activity. In some embodiments, the microbial cell is engineered relative to a parent strain. In some embodiments, the microbial cell is engineered relative to a parent strain to have a decrease in expression and/or activity of one or more enzymes involved in fatty acid activation and degradation. Additionally or alternatively, decrease in expression and/or activity of one or more β-oxidation and/or peroxisome enzymes. Additionally or alternatively, the microbial cell is engineered relative to a parent strain to have a modification in expression and/or activity of one or more lipases. Additionally or alternatively, the microbial cell is engineered relative to a parent strain to have a decrease in expression and/or activity of ω-oxidation enzymes.

In some embodiments, the microbial cell is engineered relative to a parent strain to have a decrease in expression and/or activity of one or more acyl-CoA synthetase enzymes. In some embodiments, the microbial cell is engineered relative to a parent strain to have an increase in metabolic NADPH supply. In some embodiments, the microbial cell is engineered relative to a parent strain to have a decrease in expression and/or activity of one or more neutral lipid biosynthesis enzymes. In some embodiments, the microbial cell is engineered relative to a parent strain to have a decrease in expression and/or activity of one or more of citric acid cytoplasmic exporter, and one or more NADPH dependent aldehyde reductases.

In some embodiments, the enzyme has ω-7 hydroxylase activity on C12 fatty acid substrate, and therefore is useful for biosynthesis of δ-dodecalactone from C12 fatty acid or ester or glyceride thereof. For example, in some embodiments, the enzyme is a cytochrome P450 enzyme selected from CYP505, CYP116, CYP703, and CYP102 enzyme. In some embodiments, the enzyme having fatty acid hydroxylase activity is a CYP505 enzyme. In some embodiments, the enzyme having fatty acid hydroxylase activity comprises an amino acid sequence that is at least 60% identical to the amino acid sequence selected from SEQ ID NOs: 1, 3, 4, 5, 8, and 11. In some embodiments, the enzyme having fatty acid hydroxylase activity comprises an amino acid sequence that is at least 70% identical, or at least 80% identical, or at least 90% identical, or at least 95% identical, or at least 97% identical, or at least 98% identical, or at least 99% identical to an amino acid sequence selected from SEQ ID NOs: 1, 3, 4, 5, 8, and 11.

In some embodiments, the enzyme having fatty acid hydroxylase activity comprises an amino acid sequence that is at least 50% identical, 60% identical, or at least 70% identical to the amino acid sequence of SEQ ID NO: 1. In some embodiments, the enzyme having fatty acid hydroxylase activity comprises an amino acid sequence that is at least 90% identical, or at least 95% identical, or at least 97% identical, or at least 98% identical, or at least 99% identical to an amino acid sequence of SEQ ID NO: 1.

In various embodiments, the fatty acid substrate is a C5 to C16 fatty acid. Exemplary fatty acid substrates include pentanoic acid, hexanoic acid, heptanoic acid, octanoic acid, cinnamic acid, nonaoic acid, decanoic acid, undecanoic acid, or dodecanoic acid, myristic acid, and palmitic acid, or esters (e.g., alkyl esters such as methyl esters) or glycerides thereof.

In certain embodiments, the enzymes described herein are engineered for hydroxylase activity at the desired position of a desired fatty acid substrate, to allow for production of the desired lactone.

CYP505E3 from Aspergillus terreus (SEQ ID NO: 1) is a self-sufficient P450 enzyme comprising a reductase domain, and which can catalyze in-chain hydroxylation of alkanes, fatty alcohols, and fatty acids, including at the ω-7 position. In some embodiments, the P450 enzyme comprises a domain having fatty acid hydroxylase activity, wherein the domain having fatty acid hydroxylase activity comprises an amino acid sequence that has at least 80%, or at least 90%, or at least 95%, or at least 97%, or at least 98%, or 100% sequence identity to amino acids 1 to 461 of SEQ ID NO: 1. In some embodiments, the P450 enzyme also comprises a CPR domain, which in some embodiments has at least 70% sequence identity, or at least 80% sequence identity, or at least 90% sequence identity, or at least 95% sequence identity, or at least 97% sequence identity, or at least 98% sequence identity or 100% sequence identity to a CPR domain of SEQ ID NOS: 37 to 60. In some embodiments, the CPR domain has at least 70% sequence identity, or at least 80% sequence identity, or at least 90% sequence identity, or at least 95% sequence identity, or at least 97% sequence identity, or at least 98% sequence identity or 100% sequence identity to SEQ ID NO: 53, SEQ ID NO: 57, or SEQ ID NO: 58.

In some embodiments, the P450 enzyme comprises an amino acid sequence that is at least 70%, or at least 75%, or at least 80%, or at least 85%, or at least 90%, or at least 95%, or at least 98%, or at least 99% identical to an amino acid sequence selected from SEQ ID NOs: 17 to 36. In some embodiments, the P450 enzyme comprises an amino acid sequence that is at least 70%, or at least 75%, or at least 80%, or at least 85%, or at least 90%, or at least 95%, or at least 98%, or at least 99% identical to an amino acid sequence selected from SEQ ID NOs: 29, 33, and 34.

In some embodiments, the microbial cells disclosed herein are genetically engineered to channel metabolism for maximizing the production of the one or more lactones. In some embodiments, the genetic modifications increase the availability in the microbial cells of NADPH, which is the cofactor of cytochrome P450 enzymes. In some embodiments, the genetic modifications that increase the availability of NADPH are selected from the genetic modifications that increase glycolytic flux through the oxidative pentose phosphate pathway, express an alternative route for NADPH production, and produce NADPH via tricarboxylic acid intermediates.

In some embodiments, the P450 enzyme comprises an amino acid sequence that has at least 80%, or at least 90%, or at least 95%, or at least 97%, or at least 98%, or 100% sequence identity to amino acids 1 to 461 of SEQ ID NO: 1. In some embodiments, the P450 enzyme comprises a CPR domain having at least 70% sequence identity, or at least 80% sequence identity, or at least 90% sequence identity, or at least 95% sequence identity, or at least 97% sequence identity, or at least 98% sequence identity or 100% sequence identity to a CPR domain from selected from SEQ ID NOS: 37 to 60.

In some embodiments, the P450 enzyme comprises an amino acid sequence that is at least 70%, or at least 75%, or at least 80%, or at least 85%, or at least 90%, or at least 95%, or at least 98%, or at least 99% identical to an amino acid sequence selected from SEQ ID NOs: 17 to 36. In some embodiments, the P450 enzyme comprises an amino acid sequence that is at least 70%, or at least 75%, or at least 80%, or at least 85%, or at least 90%, or at least 95%, or at least 98%, or at least 99% identical to an amino acid sequence selected from SEQ ID NOs: 29, 33, and 34.

In some embodiments, the P450 enzyme comprises an amino acid sequence that is at least 80%, or at least 85%, at least 90%, or at least 95%, or at least 97%, or at least 98%, or at least 99% identical to the amino acid sequence of SEQ ID NO: 29, and comprising one or more mutations or one or more sets of mutations listed in Table 3, 4, 5, and 6. In some embodiments, the P450 enzyme comprises a substitution of E443 with respect to SEQ ID NO: 29. In some embodiments, the P450 enzyme comprises a substitution of E443 with respect to SEQ ID NO: 29 with Gln or Asn. In some embodiments, the P450 enzyme comprises a substitution of Q53 with respect to SEQ ID NO: 29. In some embodiments, the P450 enzyme comprises a substitution of Q53 with respect to SEQ ID NO: 29 with an amino acid selected from Arg, His, and Lys. In some embodiments, the P450 enzyme comprises a substitution of P363 with respect to SEQ ID NO: 29. In some embodiments, the P450 enzyme comprises a substitution of P363 with respect to SEQ ID NO: 29 with an amino acid selected from Ala, Leu, Ile, and Val. In some embodiments, the P450 enzyme comprises a substitution of T332 with respect to SEQ ID NO: 29. In some embodiments, the P450 enzyme comprises a substitution of T332 with respect to SEQ ID NO: 29 with an amino acid selected from Ala, Leu, Ile, and Val.

In some embodiments, the P450 enzyme comprises a E443Q substitution with respect to SEQ ID NO: 29. In some embodiments, the P450 enzyme comprises a Q53R substitution with respect to SEQ ID NO: 29. In some embodiments, the P450 enzyme comprises a P363A substitution with respect to SEQ ID NO: 29. In some embodiments, the P450 enzyme comprises a T332S substitution with respect to SEQ ID NO: 29. In some embodiments, the P450 enzyme comprises a E443Q and/or Q53R substitution with respect to SEQ ID NO: 29. In some embodiments, the P450 enzyme comprises E443Q and Q53R substitutions with respect to SEQ ID NO: 29. In some embodiments, the P450 enzyme comprises one or more substitutions selected from E443Q, P363A, and Q53R with respect to SEQ ID NO: 29. In some embodiments, the P450 enzyme comprises E443Q, P363A, T332S and Q53R substitutions with respect to SEQ ID NO: 29. In some embodiments, the P450 enzyme comprises one or more substitutions selected from E443Q, T332, P363A, and Q53R with respect to SEQ ID NO: 29. In some embodiments, the P450 enzyme comprises E443Q, T332S, P363A, and Q53R substitutions with respect to SEQ ID NO: 29.

In some embodiments, the genetic modifications that maximize the production of the one or more lactones modify (increase or decrease) the amount or activity of enzymes that convert the substrates (without limitation, e.g., methyl ester of a fatty acid or triglycerides) to free fatty acids. In some embodiments, the genetic modifications that maximize the production of the one or more lactones reduce nonproductive cellular metabolism of the substrate. In some embodiments, the genetic modifications that reduce nonproductive cellular metabolism of the substrate are selected from those that decrease the amount or activity of enzymes that cause fatty acid activation and/or degradation, neutral lipid biosynthesis, β-oxidation, peroxisome function, ω-oxidation, and produce by-products.

As discussed in more details below, the microbial cell may be a yeast or fungal cell, or in some embodiments, a bacterial cell. Accordingly, the genes disrupted or inactivated according to this disclosure will depend on the species of the host. For ease of understanding, unless stated otherwise, the gene names in this disclosure are Yarrowia lipolytica genes. A person of ordinary skill will understand how to identify homologs, orthologs or paralogs for a different host species. Further, strains may be engineered for increased expression of certain genes (such as by gene complementation) or for decreased activity of certain genes (such as through expression of derivatives having loss-of-function mutation(s)), and such complementing enzymes and derivatives may generally comprise an amino acid sequence that is at least 70%, or at least 80%, or at least 90%, or at least 95%, or at least 98% identical to the reference amino acid sequence.

In some embodiments, the microbial cell is engineered relative to a parent strain to limit fatty acid activation and degradation. In some embodiments, the modifications comprise a reduction in the amount or activity of a long chain fatty acyl-CoA synthetase. In some embodiments, the modification that reduces the amount or activity of a long chain fatty acyl-CoA synthetase encoded by FAA1 gene, or a homolog, or an ortholog thereof. In some embodiments, the microbial cell comprises a partial or complete deletion of FAA1 gene, or a homolog, or an ortholog thereof.

In some embodiments, the microbial cell is engineered relative to a parent strain to cause a reduction in the amount or activity of a very long chain fatty acyl-CoA synthetase and fatty acid transporter. In some embodiments, the modification that reduces the amount or activity of a very long chain fatty acyl-CoA synthetase and fatty acid transporter encoded by FAT1 gene, or a homolog, or an ortholog thereof. In some embodiments, the microbial cell comprises a partial or complete deletion of FAT1 gene, or a homolog, or an ortholog thereof.

In some embodiments, the microbial cell is engineered relative to a parent strain to cause a reduction in the amount or activity of one or more fatty acyl-CoA synthetases. In some embodiments, the modification that reduces the amount or activity of one or more fatty acyl-CoA synthetases encoded by FAA2, FAA3, and FAA4 genes, or a homolog, or an ortholog thereof. In some embodiments, the microbial cell comprises a partial or complete deletion of FAA2, FAA3, and/or FAA4 genes, or a homolog, or an ortholog thereof.

In some embodiments, the microbial cell is engineered relative to a parent strain to cause a downregulation of β-oxidation and peroxisome metabolism. In some embodiments, the downregulation of β-oxidation and peroxisome metabolism is caused by a reduction in the amount or activity of a multifunctional β-oxidation enzyme. In some embodiments, the microbial cell comprises a modification that reduces the amount or activity of a multifunctional β-oxidation enzyme encoded by MFE1 gene, or a homolog, or an ortholog thereof. In some embodiments, the microbial cell comprises a partial or complete deletion of MFE1 gene, or a homolog, or an ortholog thereof.

In some embodiments, the microbial cell is engineered relative to a parent strain to cause a reduction in the amount or activity of one or more peroxisomal acyl-CoA oxidase. In some embodiments, the microbial cell comprises a modification that reduces the amount or activity of a peroxisomal acyl-CoA oxidase enzymes encoded by ANT1 gene, or a homolog, or an ortholog thereof. In some embodiments, the microbial cell comprises a partial or complete deletion of ANT1 gene, or a homolog, or an ortholog thereof. In some embodiments, the microbial cell comprises a modification that reduces the amount or activity of one or more peroxisomal acyl-CoA oxidase enzymes encoded by one or more of POX1, POX2, POX3, POX4, POX5, and/or POX6 genes, or homologs, or orthologs thereof. In some embodiments, the microbial cell comprises a partial or complete deletion of one or more of POX1, POX2, POX3, POX4, POX5, and/or POX6 genes, or homologs, or orthologs thereof.

In some embodiments, the microbial cell is engineered relative to a parent strain to cause a reduction in the amount or activity of a peroxisomal membrane E3 ubiquitin ligase. In some embodiments, the microbial cell is engineered relative to a parent strain to cause a reduction in the amount or activity of a peroxisomal membrane protein. In some embodiments, the microbial cell is engineered relative to a parent strain to cause a reduction in the amount or activity of a peroxisomal adenine nucleotide transporter.

In some embodiments, the microbial cell is engineered relative to a parent strain to cause a decreased expression or activity of lipases encoded by LIP2, LIP7, and/or LIP8 genes. In some embodiments, the microbial cell comprises a modification that reduces the amount or activity of the lipase encoded by LIP2 gene, or a homolog, or an ortholog thereof. In some embodiments, the microbial cell comprises a partial or complete deletion of LIP2 gene, or a homolog, or an ortholog thereof.

In alternative embodiments, the microbial cell is engineered relative to a parent strain to cause an increased expression or activity of lipases encoded by LIP2, LIP7, and/or LIP8 genes.

In some embodiments, the genetic modifications increase the metabolic supply in the microbial cells of NADPH, which is the cofactor of the fatty acid hydroxylase. In some embodiments, the microbial cell has one or more modifications that increase metabolic NADPH supply. In some embodiments, the modification(s) that increase metabolic NADPH supply (i) increase glycolytic flux through the oxidative pentose phosphate pathway; (ii) express an alternative or exogenous NADPH biosynthesis route; and/or (iii) increase production of NADPH via tricarboxylic acid intermediates.

In some embodiments, the modifications that increase the metabolic supply of NADPH increase the glycolytic flux through the oxidative pentose phosphate pathway. For example, such modifications can comprise a deletion or reduced amount or activity of: (A) glucose-6-phosphate isomerase; and/or (B) phosphofructokinase. Alternatively or in addition, the modifications that result in increased glycolytic flux through the oxidative pentose phosphate pathway may comprise an increase in the amount or activity of: (A) glucose-6-phosphate dehydrogenase; and/or (B) 6-phosphogluconate dehydrogenase. In some embodiments, the modifications that result in increased glycolytic flux through the oxidative pentose phosphate pathway comprise one or more of an overexpression of glucose-6-phosphate dehydrogenase gene (e.g., ZWF1 gene, or a homolog, or an ortholog thereof) and/or 6-phosphogluconate dehydrogenase gene (e.g., GND1 gene, or a homolog, or an ortholog thereof). In some embodiments, the modifications that increase the metabolic supply of NADPH reduce the expression or activity of glucose-6-phosphate isomerase gene (e.g., PGI1 gene, or an ortholog thereof) and/or phosphofructokinase gene (e.g., PFK1 gene, or an ortholog thereof). In some embodiments, the microbial cell belongs to the species Yarrowia lipolytica and harbors a deletion or inactivation of PGI1 and/or PFK1 gene, and/or harbors an overexpression of ZWF1 and/or GND1 (e.g., a gene complementation), or an ortholog or derivative thereof.

In some embodiments, the microbial cell is engineered to increase the metabolic supply of NADP by expressing or overexpressing an alternative or exogenous NADPH biosynthesis route. In some embodiments, the alternative or exogenous NADPH biosynthesis route comprises bacterial transhydrogenase expression, and/or a NADP-dependent glyceraldehyde-3-phosphate dehydrogenase expression. In some embodiments, the microbial cell expresses a bacterial pntAB and/or bacterial or plant gapN, or a homolog, or an ortholog thereof, or a variant thereof. In some embodiments, the microbial cell expresses a hyperactive variant of bacterial pntAB and/or bacterial or plant gapN, or a homolog, or an ortholog thereof. In some embodiments, the microbial cell belongs to the species Yarrowia lipolytica and it overexpresses bacterial pntAB and/or bacterial or plant gapN gene.

In these or other embodiments, the microbial cell has a genetic modification that results in increased production of NADPH via tricarboxylic acid intermediates. For example, the microbial cell may have an increased expression or activity of a cytosolic NADP (+)-dependent isocitrate dehydrogenase (e.g., IDH or ortholog thereof).

In some embodiments, the modifications result in reduction of nonproductive cellular metabolism of the substrates to pathways leading to, e.g., lipid storage, lipid degradation and/or formation of complex lipids. In some embodiments, the modifications limit fatty acid activation and degradation. In some embodiments, the modifications comprise a reduction in the amount or activity of: (i) long chain fatty acyl-CoA synthetase; (ii) very long chain fatty acyl-CoA synthetase and fatty acid transporter; and/or (iii) one or more fatty acyl-CoA synthetases. In some embodiments, the modifications comprise a reduction in the amount or activity of a long chain fatty acyl-CoA synthetase (e.g., the one encoded by FAA1 gene, or a homolog, or an ortholog thereof). In some embodiments, the modifications comprise a reduction in the amount or activity of a very long chain fatty acyl-CoA synthetase and fatty acid transporter (encoded by FAT1 gene, or a homolog, or an ortholog thereof). In some embodiments, the modifications comprise a reduction in the amount or activity of a fatty acyl-CoA synthetase (e.g., one or more of enzymes encoded by FAA2, FAA3 and/or FAA4 genes, or homologs, or orthologs thereof). In some embodiments, the microbial cell belongs to the species Yarrowia lipolytica and harbors a deletion or inactivation of one or more of (e.g., 2, 3, 4, or 5 of) FAA1, FAT1, FAA2, FAA3, and FAA4 genes. In some embodiments, the microbial cell belongs to the species Yarrowia lipolytica and harbors a FAA1 deletion or inactivation (encoding a long chain fatty acyl-CoA synthetase).

In some embodiments, the modifications reduce neutral lipid biosynthesis. In some embodiments, the reduction of neutral lipid biosynthesis is caused by a reduction in the amount or activity (e.g., by deletion, inactivation, or decreased expression) of: (i) diacylglycerol acyltransferase enzyme; and/or (ii) acyl-CoA:sterol acyltransferase. In some embodiments, the reduction of neutral lipid biosynthesis is caused by a reduction in the amount or activity (e.g., by deletion, inactivation, or decreased expression) of DGA1, DGA2, and/or LRO1 genes, or homologs, or orthologs thereof. In some embodiments, the reduction of neutral lipid biosynthesis is caused by a reduction in the amount or activity (e.g., by deletion, inactivation, or decreased expression) of acyl-CoA:sterol acyltransferase (e.g., encoded by ARE1 gene, or a homolog, or an ortholog thereof). In some embodiments, the reduction of neutral lipid biosynthesis is caused by a downregulation of one or more of (e.g., 2, 3, or 4 of) DGA1, DGA2, LRO1, and/or ARE1 gene, or a homolog, or an ortholog thereof. In some embodiments, the microbial cell belongs to the species Yarrowia lipolytica and harbors a deletion of one or more of DGA1, DGA2, LRO1, and ARE1 gene.

In some embodiments, the modifications downregulate β-oxidation and peroxisome metabolism. In some embodiments, the downregulation of β-oxidation and peroxisome metabolism is caused by a reduction in the amount or activity of: (i) multifunctional β-oxidation enzyme; (ii) peroxisomal membrane E3 ubiquitin ligase; (iii) peroxisomal membrane protein; (iv) one or more peroxisomal acyl-CoA oxidase; and/or (v) peroxisomal adenine nucleotide transporter. In some embodiments, the downregulation β-oxidation and peroxisome is caused by a reduction in the amount or activity of a multifunctional β-oxidation enzyme (encoded by MFE1 gene, or a homolog, or an ortholog thereof). In some embodiments, the downregulation β-oxidation and peroxisome is caused by a reduction in the amount or activity of a peroxisomal membrane E3 ubiquitin ligase (e.g., encoded by PEX10 gene, or a homolog, or an ortholog thereof). In some embodiments, the downregulation β-oxidation and peroxisome is caused by a reduction in the amount or activity of a peroxisomal membrane protein (e.g., encoded by PEXI1 gene, or a homolog, or an ortholog thereof). In some embodiments, the downregulation β-oxidation and peroxisome is caused by a reduction in the amount or activity of one or more peroxisomal acyl-CoA oxidase enzymes (e.g., encoded by one or more of POX1, POX2, POX3, POX4, POX5, and/or POX6, genes, or homologs, or orthologs thereof). In some embodiments, the downregulation β-oxidation and peroxisome is caused by a reduction in the amount or activity of a peroxisomal adenine nucleotide transporter (e.g., encoded by ANT1 gene, or a homolog, or an ortholog thereof). In some embodiments, the reduction of β-oxidation and peroxisome metabolism is caused by a deletion, inactivation, or downregulation of gene expression of one or more of (e.g., at least 2, 3, or 4 of MFE1, PEX10, PEX11, POX1, POX2, POX3, POX4, POX5, POX6, and/or ANT1 genes, or homologs, or orthologs thereof). In some embodiments, the reduction of β-oxidation and peroxisome metabolism is caused by a hypomorphic mutation or a null mutation (e.g., a deletion) in one or more of (e.g., at least 2, 3, 4 of MFE1, PEX10, PEX11, POX1, POX2, POX3, POX4, POX5, POX6, and/or ANT1 genes, or homologs, or orthologs thereof). In some embodiments, the microbial cell belongs to the species Yarrowia lipolytica and harbors a deletion or inactivation of one or more of MFE1, PEX10, PEX11, POX1, POX2, POX3, POX4, POX5, POX6, and/or ANT1 genes.

In some embodiments, the microbial cell is engineered to downregulate ω-oxidation. In some embodiments, the downregulation ω-oxidation is by a reduction in the amount or activity of one or more dodecanoic acid ω-terminal hydroxylase enzymes. In some embodiments, the downregulation of ω-oxidation is caused by a reduction in the amount or activity of dodecanoic acid ω-terminal hydroxylase encoded by one or more of ALK3, ALK4, ALK5, ALK6, and/or ALK7 genes, or homologs, or orthologs thereof. In some embodiments, the reduction of ω-oxidation is caused by a downregulation of one or more of ALK3, ALK4, ALK5, ALK6, and/or ALK7 genes, or homologs, or orthologs thereof. In some embodiments, the reduction of ω-oxidation is caused by a hypomorphic mutation, inactivation, or a null mutation (e.g., a deletion) in one or more of ALK3, ALK4, ALK5, ALK6, and/or ALK7 genes, or homologs, or orthologs thereof. In some embodiments, the microbial cell belongs to the species Yarrowia lipolytica and harbors a deletion of one or more of (e.g., 1, 2, or 3 of) ALK3, ALK4, ALK5, ALK6, and/or ALK7 genes.

In some embodiments, the microbial cell is engineered to reduce the amount or activity of enzymes producing by-products.

In Yarrowia lipolytica, citrate is exported from the cell under certain conditions. If this export is blocked, the citrate is converted to acetyl-CoA by the enzyme ATP-citrate lyase for the subsequent production of lipids. Therefore, in some embodiment, the microbial cell is engineered to reduce the amount or activity of citric acid cytoplasmic exporter (e.g., encoded by CEX1 gene, or a homolog, or and ortholog thereof). In some embodiments, the reduction in the amount or activity of citric acid cytoplasmic exporter is caused by a downregulation of one or more of CEX1 gene, or a homolog, or and ortholog thereof. In some embodiments, the reduction in the amount or activity of citric acid cytoplasmic exporter is caused by a hypomorphic mutation, inactivation, or a null mutation (e.g., a deletion) in CEX1 gene, or a homolog, or ortholog thereof. In some embodiments, the microbial cell belongs to the species Yarrowia lipolytica and harbors a deletion or inactivation of CEX1 gene.

In some embodiments, the microbial cell is engineered to reduce the amount or activity of one or more non-essential NADPH dependent aldehyde reductases. This modification is designed to decrease the consumption of NADPH, which is the cofactor for the fatty acid hydroxylase. For example, the genome of Yarrowia lipolytica contains the following putative NADPH-dependent reductases: ALR1-12 (encoded by YALI0D07634g, YALI0C13508g, YALI0F18590g, YALI0F09075g, YALI0F09097g, YALI0A15906g, YALI0C20251g, YALI0C06171g, YALI0C02805g, YALI0B15268g, YALI0B01298g, and/or YALI0B07117g See Cheng et al., Identification, characterization of two NADPH-dependent erythrose reductases in the yeast Yarrowia lipolytica and improvement of erythritol productivity using metabolic engineering, Microb Cell Fact. 17:133 (2018). In some embodiments, the reduction in the amount or activity of one or more non-essential NADPH dependent aldehyde reductases is caused by a hypomorphic mutation, inactivation, or a null mutation (e.g., a deletion) in one or more of YALI0D07634g, YALI0C13508g, YALI0F18590g, YALI0F09075g, YALI0F09097g, YALI0A15906g, YALI0C20251g, YALI0C06171g, YALI0C02805g, YALI0B15268g, YALI0B01298g, and/or YALI0B07117g genes. In some embodiments, the microbial cell belongs to the species Yarrowia lipolytica and harbors a deletion or inactivation of one or more of (e.g., 2, 3, 4, or more of) YALI0D07634g, YALI0C13508g, YALI0F18590g, YALI0F09075g, YALI0F09097g, YALI0A15906g, YALI0C20251g, YALI0C06171g, YALI0C02805g, YALI0B15268g, YALI0B01298g, and/or YALI0B07117g genes.

In some embodiments, the genetic modifications modulate the amount or activity of one or more lipases (e.g., secretory lipases). In some embodiments, the modifications comprise altered expression or activity of lipases encoded by LIP2, LIP7, and/or LIP8 genes. In some embodiments, the increased or decreased expression or activity of lipases is caused by an upregulation or downregulation of one or more of LIP2, LIP7, and/or LIP8 genes, or homologs, or orthologs thereof. In some embodiments, the increased or decreased activity of lipases is caused by a hypermorphic or hypermorphic mutation in one or more of LIP2, LIP7, and/or LIP8 genes, or homologs, or orthologs thereof. In some embodiments, the genetic modifications that increase the amount or activity of lipases comprises expression of an exogenous lipase (e.g., gene complementation). In some embodiments, one or more of LIP2, LIP7, and/or LIP8 is deleted or inactivated. In some embodiments, the microbial cell belongs to the species Yarrowia lipolytica and overexpresses one or more of LIP2, LIP7, and/or LIP8 genes or an exogenous lipase.

In other aspects, the present disclosure provides microbial cells for producing one or more lactones, where the microbial cell expresses a P450 enzyme having fatty acid hydroxylase activity. In various embodiments, the P450 enzyme comprises an amino acid sequence that has at least 80%, or at least 90%, or at least 95%, or at least 97%, or at least 98%, or 100% sequence identity to amino acids 1 to 461 of SEQ ID NO: 1, which has fatty acid hydroxylase activity (e.g., ω-7 hydroxylase activity). In various embodiments, the P450 enzyme further comprises a CPR domain having at least 70% sequence identity, or at least 80% sequence identity, or at least 90% sequence identity, or at least 95% sequence identity, or at least 97% sequence identity, or at least 98% sequence identity or 100% sequence identity to a CPR domain selected from SEQ ID NOS: 37 to 60 (e.g., SEQ ID NO: 53, SEQ ID NO: 57, or SEQ ID NO: 58).

In various embodiments, the P450 enzyme comprises an amino acid sequence that is at least 70%, or at least 75%, or at least 80%, or at least 85%, or at least 90%, or at least 95%, or at least 98%, or at least 99% identical to an amino acid sequence selected from SEQ ID NOs: 17 to 36. For example, the P450 enzyme can comprise an amino acid sequence that is at least 70%, or at least 75%, or at least 80%, or at least 85%, or at least 90%, or at least 95%, or at least 98%, or at least 99% identical to an amino acid sequence selected from SEQ ID NOs: 29, 33, and 34.

In some embodiments, the microbial cell is engineered relative to a parent strain to have one or more metabolic changes (including embodiments described above) selected from:
(a) a decrease in expression and/or activity of one or more enzymes involved in fatty acid activation and degradation,
(b) a decrease in expression and/or activity of one or more β-oxidation and/or peroxisome enzymes;
(c) a modification in expression and/or activity of one or more lipases;
(d) a decrease in expression and/or activity of ω-oxidation enzymes; and
(e) a decrease in expression and/or activity of one or more acyl-CoA synthetase enzymes;
(f) an increase in metabolic NADPH supply;
(g) a decrease in expression and/or activity of one or more neutral lipid biosynthesis enzymes; and/or
(h) a decrease in expression and/or activity of one or more of citric acid cytoplasmic exporter, and one or more NADPH dependent aldehyde reductases.

The similarity or identity of nucleotide and amino acid sequences, i.e. the percentage of sequence identity, can be determined via sequence alignments. Such alignments can be carried out with several art-known algorithms, such as with the mathematical algorithm of Karlin and Altschul (Karlin & Altschul (1993) Proc. Natl. Acad. Sci. USA 90:5873-5877), with hmmalign (HMMER package, http://hmmer.wustl.edu/) or with the CLUSTAL algorithm (Thompson, J. D., Higgins, D. G. & Gibson, T. J. (1994) Nucleic Acids Res. 22, 4673-80). The grade of sequence identity (sequence matching) may be calculated using e.g. BLAST, BLAT or BlastZ (or BlastX). A similar algorithm is incorporated into the BLASTN and BLASTP programs of Altschul et al (1990) J. Mol. Biol. 215:403-410. BLAST polynucleotide searches can be performed with the BLASTN program, score=100, word length=12.

BLAST protein searches may be performed with the BLASTP program, score=50, word length=3. To obtain gapped alignments for comparative purposes, Gapped BLAST is utilized as described in Altschul et al (1997) Nucleic Acids Res. 25:3389-3402. When utilizing BLAST and Gapped BLAST programs, the default parameters of the respective programs are used. Sequence matching analysis may be supplemented by established homology mapping techniques like Shuffle-LAGAN (Brudno M., Bioinformatics 2003b, 19 Suppl 1:154-162) or Markov random fields.

Expression of enzymes can be tuned for optimal activity, using, for example, gene modules (e.g., operons) or independent expression of the enzymes. For example, expression of the genes can be regulated through selection of promoters, such as inducible or constitutive promoters, with different strengths (e.g., strong, intermediate, or weak). Additionally, expression of genes can be regulated through manipulation of the copy number of the gene in the cell. In some embodiments, expression of genes can be regulated through manipulating the order of the genes within a module, where the genes transcribed first in an operon are generally expressed at a higher level. In some embodiments, expression of genes is regulated through integration of one or more genes into the chromosome.

Optimization of expression can also be achieved through selection of appropriate promoters and ribosomal binding sites. In some embodiments, this may include the selection of high-copy number plasmids, or single-, low- or medium-copy number plasmids. The step of transcription termination can also be targeted for regulation of gene expression, through the introduction or elimination of structures such as stem-loops.

Expression vectors containing all the necessary elements for expression are commercially available and known to those skilled in the art. See, e.g., Sambrook et al., Molecular Cloning: A Laboratory Manual, Second Edition, Cold Spring Harbor Laboratory Press, 1989. Cells are genetically engineered by the introduction into the cells of heterologous DNA. The heterologous DNA is placed under operable control of transcriptional elements to permit the expression of the heterologous DNA in the host cell.

In some embodiments, endogenous genes are edited, as opposed to gene complementation. Editing can modify endogenous promoters, ribosomal binding sequences, or other expression control sequences, and/or in some embodiments modifies trans-acting and/or cis-acting factors in gene regulation. Genome editing can take place using CRISPR/Cas genome editing techniques, or similar techniques employing zinc finger nucleases and TALENs. In some embodiments, the endogenous genes are replaced by homologous recombination.

In some embodiments, genes are overexpressed at least in part by controlling gene copy number. While gene copy number can be conveniently controlled using plasmids with varying copy number, gene duplication and chromosomal integration can also be employed. For example, a process for genetically stable tandem gene duplication is described in US 2011/0236927, which is hereby incorporated by reference in its entirety.

In accordance with this disclosure, where genes are deleted, genes can be deleted in whole or in part (i.e., inactivated), which can include deletion of coding sequences and/or expression control sequences.

In some embodiments, the lactone produced according to this disclosure is derivable from a C₅ to C₁₆ aliphatic fatty acid or C₆ to C₁₆ aromatic carboxylic acid or an alkyl (e.g., methyl) ester or glyceride thereof. In various embodiments, the lactone is a γ-lactone or a δ-lactone. In some embodiments, the lactone is selected from γ-pentalactone (C₅H₈O₂), γ-hexalactone (C₆H₁₀O₂), γ-heptalactone (C₇H₁₂O₂), γ-octalactone (C₈H₁₄O₂), δ-octalactone (C₈H₁₄O₂), coumarin (C₉H₆O₂), γ-nonalactone (C₉H₁₆O₂), δ-nonalactone (C₉H₁₆O₂), γ-decalactone (C₁₀H₁₈O₂), δ-decalactone (C₁₀H₁₈O₂), γ-undecalactone (C₁₁H₂₀O₂), δ-undecalactone (C₁₁H₂₀O₂), γ-dodecalactone (C₁₂H₂₂O₂), and δ-dodecalactone (C₁₂H₂₂O₂).

In some embodiments, the microbial cell is a yeast or fungal cell. In some embodiments, the microbial cell is an oleaginous yeast (without limitation, e.g., Yarrowia lipolytica). In some embodiments, the yeast or fungal cell belongs to a genus selected from Ashbya, Aspergillus, Aurantiochytrium, Bastobotyrs, Candida, Claviceps, Cryptococcus, Cunninghamella, Geotrichum, Hansenula, Issatchenkia, Kluyveromyces, Kodamaea, Leucosporidiella, Linderna, Lipomyces, Mortierella, Myxozyma, Mucor, Occultifur, Ogataea, Penicillium, Phaffia, Pichia, Prototheca, Rhizopus, Rhodosporidium, Rhodotorula, Saccharomyces, Scheffersomyces, Schizosaccharomyces, Sporidiobolus, Sporobolomyces, Starmerella, Tremella, Trichosporon, Wickerhamomyces, Waltomyces, and Yarrowia. In some embodiments, the yeast or fungal cell belongs to a species selected from Yarrowia lipolytica, Yarrowia phangngensis, Pichia kudriavzevii, Saccharomyces cerevisiae, Pichia pastoris, Kluyveromyces marxianus, Rhodosporidium toruloides, Sporidiobolus ruinenii, Sporidiobolus salmonicolor, Aspergillus oryzae, Mortierella isabellina, Waltomyces lipofer, Candida tropicalis, Candida boidinii, Scheffersomyces stipitis, Mucor circinelloides, Ashbya gossypii, Trichoderma harzianum, Pichia guilliermondii, Kodamaea ohmeri, Rhodotorula aurantiaca, Lindnera saturnus, Penicillium roqueforti, Lipomyces starkeyi, and Bastobotyrs adeninivorans. In some embodiments, the yeast or fungal cell is Yarrowia lipolytica.

In some embodiments, the microbial cell is a bacterial cell. In some embodiments, the microbial cell is a bacterium that accumulates significant quantities of triacylglycerols (TAGs); without limitation, Rhodococcus opacus, Acinetobacter calcoaceticus, Streptomyces coelicolor, Rhodococcus jostii, and Acinetobacter baylyi). In some embodiments, the bacterial cell belongs to a genus selected from Acidovorax, Acinetobacter, Actinomyces, Alcanivorax, Arthrobacter, Brevibacterium, Bacillus, Clostridium, Corynebacterium, Dietzia, Escherichia, Gordonia, Marinobacter, Mycobacterium, Micrococcus, Micromonospora, Moraxella, Nocardia, Pseudomonas, Psychrobacter, Rhodococcus, Salmonella, Streptomyces, Thalassolituus, and Thermomonospora. In some embodiments, the bacterial cell belongs to a species selected from Rhodococcus opacus, Acinetobacter calcoaceticus, Streptomyces coelicolor, Rhodococcus jostii, and Acinetobacter baylyi.

In some embodiments, the host cell is Yarrowia lipolytica having one or more genetic modifications increasing the availability of NADPH described herein. To improve availability of NADPH for hydroxylation of fatty acid, a series of gene knock-outs and gene insertions can be introduced to increase the availability of NADPH. For example, genetic modifications can increase glycolytic flux through the oxidative pentose phosphate pathway, express an alternative or exogenous NADPH biosynthesis route; and/or increase production of NADPH via tricarboxylic acid intermediates. Another series of knock-outs can reduce the utilization of NADPH in other non-essential pathways.

The host cells and methods are further suitable for commercial production of lactone, that is, the cells and methods can be productive at commercial scale. In some embodiments, the size of the culture is at least about 100 L, at least about 200 L, at least about 500 L, at least about 1,000 L, or at least about 10,000 L. In an embodiment, the culturing may be conducted in batch culture, continuous culture, or semi-continuous culture.

In one aspect, the present disclosure provides a method for making a lactone. The method comprises culturing a microbial cell of any of the embodiments disclosed herein with a fatty acid substrate or an ester thereof (e.g., alkyl ester such as a methyl ester or ethyl ester) or glyceride (e.g., mono-, di-, or triglyceride) thereof, and recovering the lactone from the culture. In other embodiments, the fatty acid substrate is produced by the cell, which can comprise expression of a biosynthetic pathway comprising one or more heterologous enzymes. In some embodiments, the one or more lactones produced according to this disclosure are selected from γ-pentalactone (C₅H₈O₂), γ-hexalactone (C₆H₁₀O₂), γ-heptalactone (C₇H₁₂O₂), γ-octalactone (C₈H₁₄O₂), δ-octalactone (C₈H₁₄O₂), coumarin (C₉H₆O₂), γ-nonalactone (C₉H₁₆O₂), δ-nonalactone (C₉H₁₆O₂), γ-decalactone (C₁₀H₁₈O₂), δ-decalactone (C₁₀H₁₈O₂), γ-undecalactone (C₁₁H₂₀O₂), δ-undecalactone (C₁₁H₂₀O₂), γ-dodecalactone (C₁₂H₂₂O₂), and δ-dodecalactone (C₁₂H₂₂O₂). In some embodiments, the fatty acid substrate is C₅ to C₁₆ aliphatic fatty acid or C₆ to C₁₆ aromatic carboxylic acid or an alkyl ester or glyceride thereof.

In some embodiments, (i) the lactone is γ-pentalactone (C₅H₈O₂) and the substrate is pentanoic acid, or an ester or glyceride thereof; (ii) the lactone is γ-hexalactone (C₆H₁₀O₂) and the substrate is hexanoic acid, or an ester or glyceride thereof; (iii) the lactone is γ-heptalactone (C₇H₁₂O₂) and the substrate is heptanoic acid, or an ester or glyceride thereof; (iv) the lactone is γ-octalactone (C₈H₁₄O₂) or δ-octalactone (C₈H₁₄O₂), and the substrate is octanoic acid, or an ester or glyceride thereof; (v) the lactone is coumarin (C₉H₆O₂) and the substrate is cinnamic acid, or an ester or glyceride thereof; (vi) the lactone is γ-nonalactone (C₉H₁₆O₂) or δ-nonalactone (C₉H₁₆O₂), and the substrate is nonaoic acid, or an ester or glyceride thereof; the lactone is γ-decalactone (C₁₀H₁₈O₂) or δ-decalactone (C₁₀H₁₈O₂), and the substrate is decanoic acid, or an ester or glyceride thereof; the lactone is γ-undecalactone (C₁₁H₂₀O₂) or δ-undecalactone (C₁₁H₂₀O₂), and the substrate is undecanoic acid, or an ester or glyceride thereof; or the lactone is γ-dodecalactone (C₁₂H₂₂O₂) or δ-dodecalactone (C₁₂H₂₂O₂), and the substrate is dodecanoic acid, or an ester or glyceride thereof.

In some embodiments, the lactone is δ-dodecalactone (C₁₂H₂₂O₂), and the substrate is dodecanoic acid, or an ester (e.g., an alkyl ester such as methyl or ethyl ester) or glyceride thereof. In some embodiments, the δ-dodecalactone comprises at least 50%, or at least 60%, or at least 75%, or at least 80% or at least 90% R-δ-dodecalactone.

In some aspects, the invention provides methods for making a product comprising a lactone ingredient, which is δ-dodecalactone in some embodiments. The method comprises culturing a strain described herein that produces one or more lactones, recovering the lactone(s), and incorporating the lactone(s) into a product. Examples of products in which highly purified target lactone(s) may be used include, but are not limited to, perfumes, cosmetics, food products, beverages, flavors, food additives, fragrances, detergent fragrances, green solvents, antimicrobial ingredients, polymers, nylon precursors, and fuel precursors.

In one aspect, the present disclosure provides a method for making a product, comprising, incorporating a lactone made according to the method of any of the embodiments disclosed herein into said product. In some embodiments, the product is selected from a perfume, a cosmetic, a food product, a beverage, a flavor, a food additive, a fragrance, a detergent fragrance, a green solvent, an antimicrobial ingredient, a polymer, a nylon precursor, and a fuel precursor.

During the manufacturing of products such as foodstuffs, drinks, pharmaceuticals, and cosmetics, conventional methods such as mixing, kneading, dissolution, pickling, permeation, percolation, sprinkling, atomizing, infusing and other methods may be used.

In still other aspects, the present disclosure provides P450 enzymes having fatty acid hydroxylase activity. In some embodiments, the P450 enzyme comprises an amino acid sequence that has at least 80% sequence identity to amino acids 1 to 461 of SEQ ID NO: 1, and comprises a CPR domain having at least 70% sequence identity to a CPR domain selected from SEQ ID NOS: 37 to 60 (e.g., SEQ ID NO: 53, SEQ ID NO: 57, or SEQ ID NO: 58). In various embodiments, the P450 enzyme comprises an amino acid sequence that has at least 80% sequence identity, or at least 90% sequence identity, or at least 95% sequence identity, or at least 97% sequence identity, or at least 98% sequence identity to SEQ ID NO: 1. In some embodiments, the P450 enzyme comprises the amino acids sequence of 1 to 461 of SEQ ID NO: 1, and a CPR domain.

In some embodiments, the P450 enzyme comprises a CPR domain that has at least 80%, or at least 85%, or at least 90%, or at least 95%, or at least 97%, or at least 98% sequence identity to a CPR domain selected from SEQ ID NOS: 37 to 60. For example, the P450 enzyme may comprise a CPR domain that has at least 80%, or at least 85%, or at least 90%, or at least 95%, or at least 97%, or at least 98% sequence identity to a CPR domain from SEQ ID NO: 53, SEQ ID NO: 57, and SEQ ID NO: 58.

In exemplary embodiments, the P450 enzyme comprises an amino acid sequence that has at least 70% sequence identity, or at least 80% sequence identity, or at least 90% sequence identity, or at least 95% sequence identity, or at least 97% sequence identity, or at least 98% sequence identity to any one of SEQ ID NOS: 17 to 36. For example, the P450 enzyme may comprise an amino acid sequence that has at least 70% sequence identity, or at least 80% sequence identity, or at least 90% sequence identity, or at least 95% sequence identity, or at least 97% sequence identity, or at least 98% sequence identity to any one of SEQ ID NOS: 29, 33, and 34.

In still other aspects, the invention provides a polynucleotide encoding the P450 enzyme disclosed herein, and which may include expression control sequences, such as a promoter operably positioned to direct expression of the polynucleotide in a host cell. The invention therefore further provides host cells expressing the polynucleotide, which can include bacterium (e.g., E. coli) or yeast, including host cells genus and species described herein for lactone production.

Aspects and embodiments of the invention will now be described according to the following examples,

### EXAMPLES

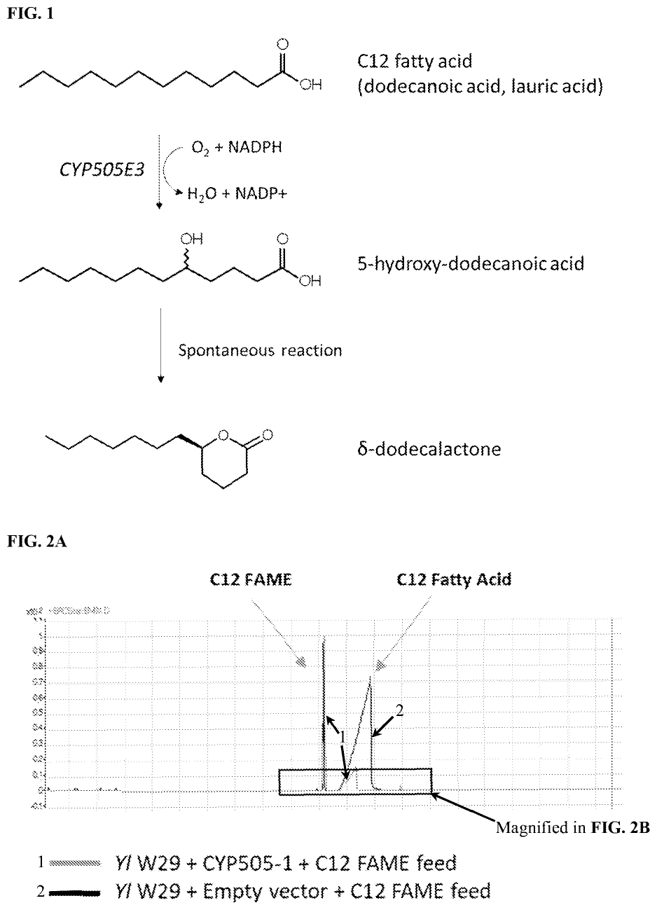

δ-dodecalactone (FIG. 1) is a derivative of the C12 fatty acid, lauric acid (dodecanoic acid), and is a flavoring/aromatic agent that imparts fatty, creamy, fruity flavor or fragrance to food, feed, and cosmetics. The biosynthesis of δ-dodecalactone according to the present disclosure involves hydroxylation of C12 fatty acid followed by a spontaneous cyclization reaction (FIG. 1).

### Example 1. Yarrowia lipolytica Strains Expressing a Cytochrome P450 Enzyme Fed with C-12 Fatty Acid Methyl Ester (FAME) Produce δ-Dodecalactone

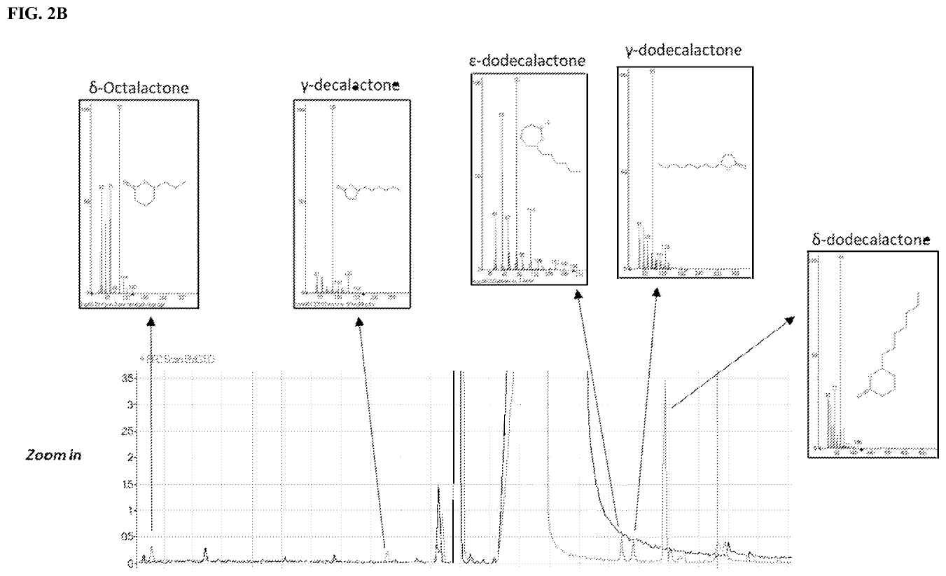

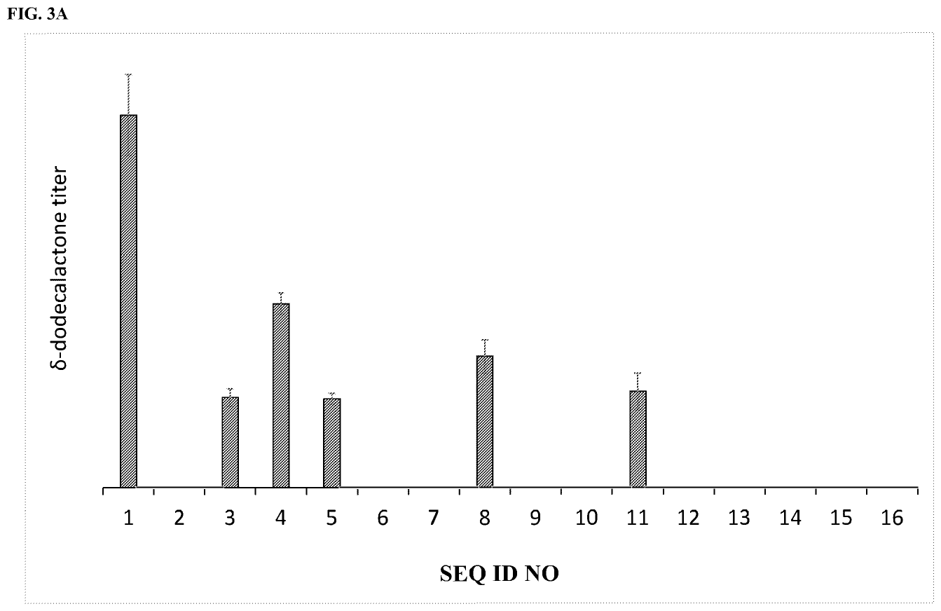

The Aspergillus terreus cytochrome P450 enzyme (CYP505-1; SEQ ID NO: 1) was expressed in Yarrowia lipolytica, and the fatty-acid subterminal (ω-7) hydroxylase activity of the enzyme was tested with whole cell extraction. A control Yarrowia lipolytica strain harboring an empty vector and a Yarrowia lipolytica strain overexpressing CYP505-1 (SEQ ID NO: 1) were grown in media containing glucose and C12 fatty acid methyl ester (FAME) for 72 hrs at 30° C. After the fermentation, whole cultures (cells and broth) were extracted with isooctane and analyzed by GC-MS. As shown in FIG. 2A, the GC-MS profile of the control strain exhibited mostly C12 FAME and C12 fatty acids. On the other hand, the GC-MS profile of CYP505-1 (SEQ ID NO: 1)-overexpressing strain showed additional products. To see the subterminal hydroxylation products clearly, the area of FIG. 2A bracketed with a rectangle was magnified. As shown in FIG. 2B, the CYP505-1 (SEQ ID NO: 1)-overexpressing strain produced δ-dodecalactone. Detectable amounts of δ-octalactone, γ-decalactone, ¿-dodecalactone, and γ-dodecalactone were also observed.

### Example 2: Screening Cytochrome P450 Enzymes for the Biosynthesis of δ-Dodecalactone

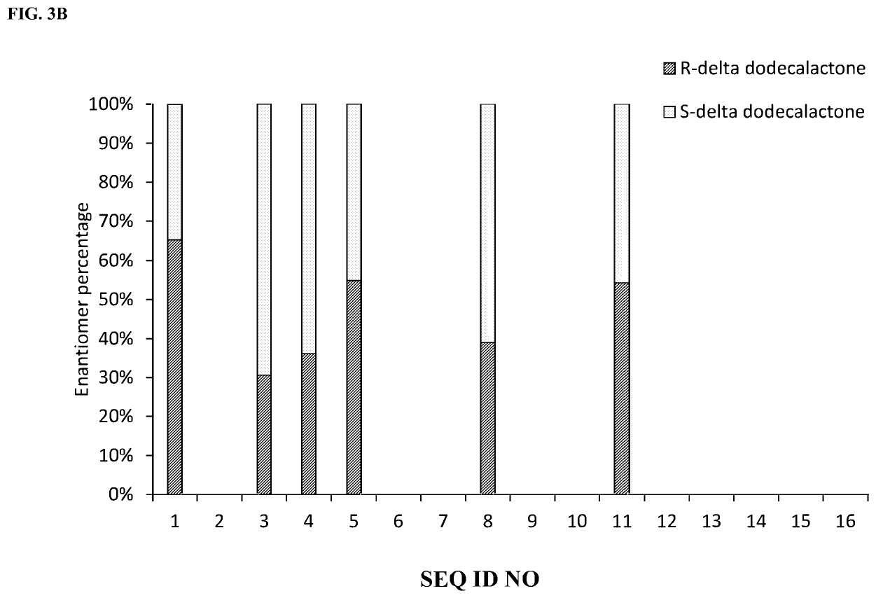

Cytochrome P450 (CYP) enzymes were screened for their ω-7 hydroxylase activity, which leads to the formation of δ-dodecalactone. The CYP enzymes shown in Table 1 below were expressed in Yarrowia lipolytica (FIG. 3A).

TABLE 1
Cytochrome P450 Enzymes

| Family | Organism | Gene | SEQ ID NO: |
|---|---|---|---|
| CYP505 | Aspergillus terreus | CYP505-1 | 1 |
| | Aspergillus niger | CYP505-2 | 2 |
| | Aspergillus luchuensis | CYP505-3 | 3 |
| | Penicillium expansum | CYP505-4 | 4 |
| | Aspergillus tubingensis | CYP505-5 | 5 |
| | Penicillium camemberti | CYP505-6 | 6 |
| | Penicillium freii | CYP505-7 | 7 |
| | Aspergillus brasiliensis | CYP505-8 | 8 |
| | Aspergillus aureoterreus | CYP505-9 | 9 |
| | Penicillium canescens | CYP505-10 | 10 |
| | Aspergillus alliaceus | CYP505-11 | 11 |
| CYP116 | Tepidiphilus thermophilus | CYP116-1 | 12 |
| | Albidovulum xiamenense | CYP116-2 | 13 |
| | Jhaorihella thermophile | CYP116-3 | 14 |
| CYP703 | Arabidopsis thaliana | CYP703-1 | 15 |
| CYP102 | Bacillus megaterium | CYP102-1 | 16 |

Yarrowia lipolytica expressing one of the above enzymes were grown in media containing glucose and C12 FAME for 72 hrs at 30° C. After the fermentation, whole cultures (cells and broth) were extracted with isooctane and analyzed by GC-MS, and relative titers of δ-dodecalactone were determined. As shown in FIG. 3A, Yarrowia lipolytica strain expressing CYP505 enzymes of SEQ ID NOs: 1, 3-5, 8 and 11 produced δ-dodecalactone. R-δ-dodecalactone and S-δ-dodecalactone enantiomers were separately quantified. As shown in FIG. 3B, Yarrowia lipolytica strain expressing CYP505 enzymes of SEQ ID NOs: 1, 3-5, 8 and 11 produced both R-δ-dodecalactone and S-δ-dodecalactone enantiomers to varying degrees. The enzyme of SEQ ID NO: 1 produced the highest amount of the R enantiomer.

### Example 3: Increased Production of Lactone by Yarrowia lipolytica FAA14 Strains

The yield of a lactone such as δ-dodecalactone may be increased by certain genetic modifications that limit consumption of the fatty acid substrate. One of these modifications is reduced expression or activity of the long chain fatty acyl-CoA synthetase FAA1. To test this strategy, Yarrowia lipolytica FAA1Δ strains were constructed, and subsequently transformed with an expression vector directing expression of CYP505-1 (SEQ ID NO: 1) (pY4-CYP505E3) or an empty control vector.

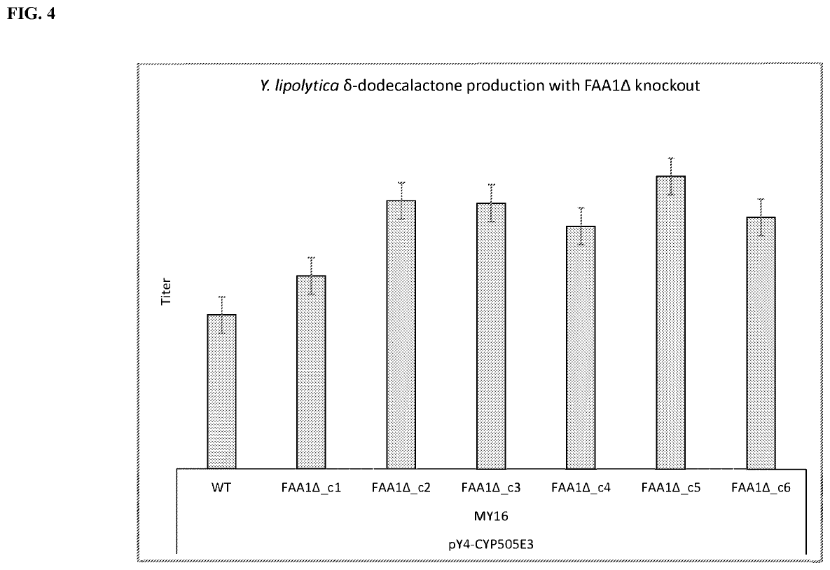

Wild type Yarrowia lipolytica or six colonies of the Yarrowia lipolytica FAA14 strain harboring the expression vector or the control vector were pre-cultured for 24 hours in medium containing 20 g/L glucose at 30° C. and 900 rpm in a 96 deep well plate. The pre-culture was used to inoculate a second deep well plate with medium containing 20 g/L glucose incubated in the same conditions. After 24 hours, methyl laurate (C12 FAME) was added at a concentration of 40 g/L C12 FAME. The fermentation continued for an additional 48 hours. Whole culture was extracted with isooctane and analysis of the organic phase by gas chromatography was performed to quantify the amount of δ-dodecalactone produced. As shown in FIG. 4, each of the six colonies of the Yarrowia lipolytica FAA14 strain expressing CYP505 enzyme produced more δ-dodecalactone compared to wild type Yarrowia lipolytica strain expressing CYP505 enzyme. Both the wild type Yarrowia lipolytica strain and all six colonies of the Yarrowia lipolytica FAA14 strain harboring the pY4 empty control vector produced only a background signal for δ-dodecalactone.

### Example 4: Engineering of CYP505 Enzymes

To engineer CYP 505, the reductase domain of the CYP505E3 enzyme (SEQ ID NO: 1) was swapped with other reductase domains to generate chimeric enzymes. Chimeric enzymes are listed in the following Table 2.

TABLE 2
Chimeric CYP505 Enzymes

| Gene | CPR domain origin | SEQ# |
|---|---|---|
| CYP505E3_1 | Native CPR | 1 |
| CYP505E3_XP_018663495.1 | XP_018663495.1 | 17 |
| CYP505E3_GFP54330.1 | GFP54330.1 | 18 |
| CYP505E3_PTB78938.1 | PTB78938.1 | 19 |
| CYP505E3_KIN03265.1 | KIN03265.1 | 20 |
| CYP505E3_PVH93311.1 | PVH93311.1 | 21 |
| CYP505E3_XP_007693841.1 | XP_007693841.1 | 22 |
| CYP505E3_XP_014076579.1 | XP_014076579.1 | 23 |
| CYP505E3_XP_007697425.1 | XP_007697425.1 | 24 |
| CYP505E3_XP_016765520.1 | XP_016765520.1 | 25 |
| CYP505E3_XP_035319610.1 | XP_035319610.1 | 26 |
| CYP505E3_KAF2023181.1 | KAF2023181.1 | 27 |
| CYP505E3_KAF3072637.1 | KAF3072637.1 | 28 |
| CYP505E3_EWZ46970.1 | EWZ46970.1 | 29 |
| CYP505E3_KDN62764.1 | KDN62764.1 | 30 |
| CYP505E3_ROW04469.1 | ROW04469.1 | 31 |
| CYP505E3_KAF2108562.1 | KAF2108562.1 | 32 |
| CYP505E3_ORY02372.1 | ORY02372.1 | 33 |
| CYP505E3_KAF2196115.1 | KAF2196115.1 | 34 |
| CYP505E3_XP_018183292.1 | XP_018183292.1 | 35 |
| CYP505E3_XP_029748982.1 | XP_029748982.1 | 36 |

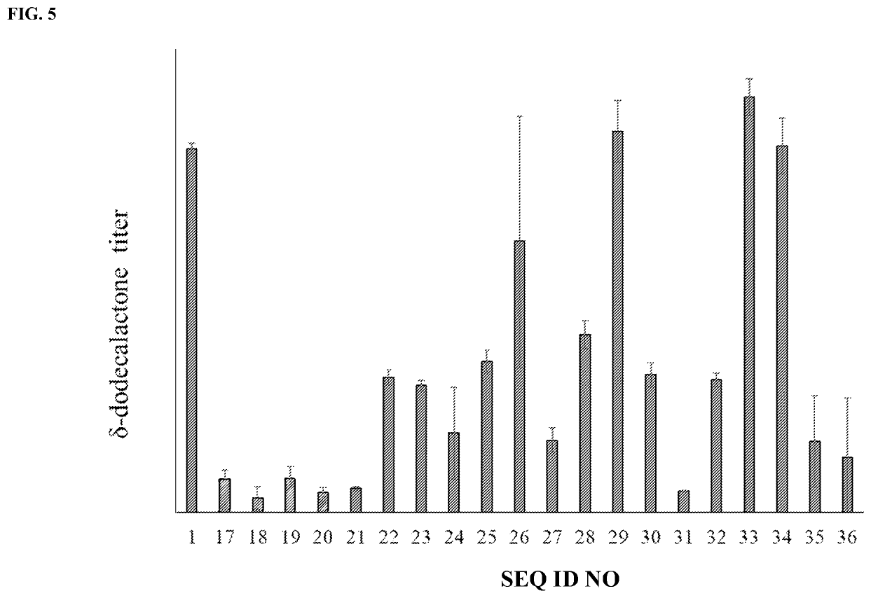

Chimeras were cloned into pY4 expression vector, and Yarrowia lipolytica USDA-2 strain was transformed with the expression vectors. Yarrowia lipolytica USDA-2 strain expressing CYP505-1 (SEQ ID NO: 1) (pY4-CYP505E3) was used as a control. These strains were grown in media containing glucose and C12 fatty acid methyl ester (FAME) for 72 hrs at 30° C. Whole culture was extracted with isooctane and analysis of the organic phase by gas chromatography was performed to quantify the amount of δ-dodecalactone produced. As shown in FIG. 5, each of the Yarrowia lipolytica strains expressing each of the chimeras produced detectable amounts of δ-dodecalactone. The Yarrowia lipolytica strains expressing SEQ ID NOs. 29, 33 and 34 produced δ-dodecalactone in amounts that were at least comparable to (and actually greater than in this study) the amount produced by Yarrowia lipolytica strain expressing CYP505-1 (SEQ ID NO: 1) (FIG. 5). The Yarrowia lipolytica strains expressing SEQ ID NOs. 22, 23, 24, 25, 26, 27, 28, 30 and 32 also produced significant amounts of δ-dodecalactone (FIG. 5)

These results demonstrate that the chimeras disclosed herein have fatty-acid hydroxylase activity, including subterminal (ω-7) hydroxylase activity. These chimeric enzymes provide opportunities to engineer cytochrome P450 enzymes having advantages in lactone biosynthesis, including advantages in product yield (including product stereochemistry), substrate selectivity, level of expression and stability in microbial hosts, among others.

### Example 5: Engineering of Cytochrome P450 for Laurate ω-7 Hydroxylase (LAH) Activity

The chimeric enzyme of SEQ ID NO: 29, which has the cytochrome P450 reductase (CPR) domain from the bifunctional P-450: NADPH-P450 reductase from Fusarium oxysporum (Fo47, referred herein as “LAH_0”) was further engineered. Numerous derivatives of LAH_0 (SEQ ID NO: 29) having various amino acid substitutions were constructed. Yarrowia lipolytica strains expressing each of the mutant derivatives were grown in media containing glucose and C12 fatty acid methyl ester (FAME) for 72 hrs at 30° C. Whole culture was extracted with isooctane and analysis of the organic phase by gas chromatography was performed to quantify the amount of δ-dodecalactone produced. Effects of the amino acid substitutions were evaluated by comparing the titers of δ-dodecalactone produced. Table 3 shows thirty-three amino acid substitutions that showed beneficial changes in the titers of δ-dodecalactone compared to LAH_0:

TABLE 3
Round 1 of LAH Engineering

| Mutant | Fold Improvement δ-dodecalactone |
|---|---|
| E443Q | 1.59 |
| Q128S | 1.37 |
| I46L | 1.34 |
| V126I | 1.31 |
| S139P | 1.30 |
| E121D | 1.27 |
| L43I | 1.27 |
| I202L | 1.26 |
| R98P | 1.25 |
| F40Y | 1.25 |
| I145V | 1.20 |
| A349K | 1.20 |
| T427N | 1.19 |
| L242M | 1.19 |
| A411P | 1.15 |
| T57S | 1.14 |
| M76T/S193A | 1.13 |
| N49D/G197R | 1.12 |
| V235K | 1.11 |
| Q287K | 1.10 |
| Q8P | 1.10 |
| V249K | 1.10 |
| A56V | 1.10 |
| P42E | 1.08 |
| R294K | 1.06 |
| V108M | 1.06 |
| G29P | 1.04 |
| R315K | 1.04 |
| I322V | 1.04 |
| D257E | 1.03 |
| A232P | 1.03 |
| G223E | 1.02 |

Based on the above results, SEQ ID NO: 29 derivative having E443Q substitution was chosen as lead laurate ω-7 hydroxylase enzyme, LAH_1 (SEQ ID NO: 61). Numerous derivatives of LAH_1 (SEQ ID NO: 61) having various amino acid substitutions were constructed. Yarrowia lipolytica strains expressing each of the mutant derivatives were grown in media containing glucose and C12 fatty acid methyl ester (FAME) for 72 hrs at 30° C. Whole culture was extracted with isooctane and analysis of the organic phase by gas chromatography was performed analyze the results. Effects of the amino acid substitutions were evaluated by comparing the titers of total amount of δ-dodecalactone produced and the fraction of % (R)-δ-dodecalactone with in the total δ-dodecalactone. Table 4 shows forty-six amino acid substitutions that showed beneficial changes in the titers of δ-dodecalactone and/or selectivity for the R-isomer compared to LAH_1:

TABLE 4
Round 2 of LAH Engineering

| | Fold Improvement | Fold Improvement |
|---|---|---|
| | total δ- | in % (R)-δ- |
| Mutant | dodecalactone | dodecalactone |
| F40Y/I46L/Q128S | 1.45 | 1.02 |
| L154F | 1.34 | 1.06 |
| F40Y/I145V/A349K/T427N | 1.33 | 1.06 |
| I46L | 1.30 | 1.03 |
| T427N | 1.24 | 1.01 |
| A349K | 1.24 | 1.01 |
| A349R | 1.23 | 1.01 |
| A349Q | 1.15 | 0.98 |
| A349K/T427N | 1.15 | 1.05 |
| L242I | 1.15 | 1.04 |
| L43I | 1.12 | 1.02 |
| I46L/I145V | 1.10 | 1.04 |
| I54L | 1.09 | 1.03 |
| Q137H | 1.09 | 1.07 |
| F40Y | 1.08 | 1.03 |
| N244Q | 1.05 | 0.97 |
| Q53I | 1.05 | 1.06 |
| Q128N | 1.05 | 1.03 |
| R98K | 1.04 | 1.01 |
| F282M | 1.04 | 0.97 |
| Q53L | 1.03 | 0.98 |
| G99N | 1.03 | 1.05 |
| L43I/V126I/S139P/I202L | 1.02 | 1.02 |
| Q128S | 1.02 | 1.03 |
| I145V | 1.02 | 1.04 |
| Q128T | 1.02 | 1.06 |
| Q137F | 1.02 | 1.07 |
| I260M | 1.01 | 1.03 |
| R98A | 1.01 | 0.98 |
| N244D | 1.01 | 0.95 |
| S255T | 1.00 | 0.98 |
| S139P | 1.00 | 0.99 |
| T146A | 0.98 | 1.04 |
| Q53R | 0.98 | 1.15 |
| G101A | 0.95 | 1.00 |
| G99S/Q426K | 0.92 | 1.13 |
| A351P | 0.91 | 1.01 |
| F44Y | 0.88 | 1.02 |
| L242A | 0.85 | 1.07 |
| L43I/E121D/Q128S/I202L | 0.85 | 1.04 |
| L107I | 0.84 | 1.08 |
| S87A | 0.83 | 1.04 |
| G99T | 0.77 | 1.03 |
| T153A | 0.73 | 1.00 |
| L242F | 0.67 | 1.00 |
| L107V | 0.66 | 1.10 |

Based on the above results, SEQ ID NO: 61 derivative having Q53R substitution was chosen as lead laurate ω-7 hydroxylase enzyme, LAH_2 (SEQ ID NO: 62). Numerous derivatives of LAH_2 (SEQ ID NO: 62) having various amino acid substitutions were constructed. Yarrowia lipolytica strains expressing each of the mutant derivatives were grown in media containing glucose and C12 fatty acid methyl ester (FAME) for 72 hrs at 30° C. Whole culture was extracted with isooctane and analysis of the organic phase by gas chromatography was performed analyze the results. Effects of the amino acid substitutions were evaluated by comparing the titers of total amount of δ-dodecalactone produced and the fraction of % (R)-δ-dodecalactone with in the total δ-dodecalactone. Table 4 shows fifty-five amino acid substitutions that showed beneficial changes in the titers of δ-dodecalactone and/or selectivity for the R-isomer compared to LAH_2:

TABLE 5
Round 3 of LAH Engineering

| | Fold Improvement | Fold Improvement in % |
|---|---|---|
| Mutant | total δ-dodecalactone | (R)-δ-dodecalactone |
| G78A | 1.45 | 0.80 |
| M84A | 1.22 | 1.00 |
| C32A | 1.20 | 1.05 |
| H28A | 1.16 | 1.02 |
| E80A | 1.15 | 0.97 |
| E31A | 1.15 | 1.04 |
| I34A | 1.15 | 0.99 |
| K81A | 1.14 | 0.91 |
| L30A | 1.14 | 1.08 |
| C365A | 1.14 | 1.02 |
| P340A | 1.14 | 0.92 |
| A335G | 1.14 | 1.06 |
| E27A | 1.12 | 1.05 |
| I268A | 1.11 | 0.85 |
| A417G | 1.10 | 0.93 |
| R222A | 1.08 | 1.04 |
| V364A | 1.07 | 0.98 |
| K341A | 1.05 | 1.05 |
| D257S | 1.05 | 1.06 |
| Q426K | 1.04 | 1.03 |
| Q45A | 1.04 | 1.08 |
| L216A | 1.03 | 1.00 |
| L154F | 1.02 | 1.06 |
| K442A | 1.02 | 0.97 |
| S193A | 1.02 | 0.96 |
| A357G | 1.01 | 0.98 |
| T57A/E64K | 1.01 | 1.04 |
| D147A | 1.00 | 1.04 |
| H213A | 0.99 | 0.90 |
| Q215A | 0.99 | 1.05 |
| P363A | 0.98 | 1.24 |
| G355A | 0.98 | 1.00 |
| K52A | 0.94 | 1.02 |
| T161A | 0.92 | 1.03 |
| S58A | 0.92 | 1.03 |
| D256A | 0.91 | 1.00 |
| V185A | 0.91 | 1.01 |
| V360A | 0.90 | 1.04 |
| Q443A | 0.90 | 1.02 |
| D25A | 0.86 | 1.04 |
| G99S | 0.86 | 1.11 |
| N219A | 0.86 | 1.01 |
| A221A | 0.85 | 1.01 |
| T281A | 0.78 | 1.01 |
| S255A | 0.77 | 1.01 |
| T156A | 0.76 | 1.05 |
| M210A | 0.71 | 1.01 |
| M254A | 0.68 | 1.02 |
| I48A | 0.65 | 1.00 |
| S275A | 0.65 | 1.00 |
| T332A | 0.64 | 1.07 |
| N261A | 0.55 | 1.01 |
| L366A | 0.33 | 1.00 |
| L445A | 0.21 | 1.43 |
| L186A | 0.05 | 1.20 |

Based on the above results, SEQ ID NO: 62 derivative having P363A substitution was chosen as the next lead laurate ω-7 hydroxylase enzyme, LAH_3 (SEQ ID NO: 63). Numerous derivatives of LAH_3 (SEQ ID NO: 63) having various amino acid substitutions were constructed. Yarrowia lipolytica strains expressing each of the mutant derivatives were grown in media containing glucose and C12 fatty acid methyl ester (FAME) for 72 hrs at 30° C. Whole culture was extracted with isooctane and analysis of the organic phase by gas chromatography was performed analyze the results. Effects of the amino acid substitutions were evaluated by comparing the titers of total amount of δ-dodecalactone produced and the fraction of % (R)-δ-dodecalactone with in the total δ-dodecalactone. Table 4 shows fifty-five amino acid substitutions that showed beneficial changes in the titers of δ-dodecalactone and/or selectivity for the R-isomer compared to LAH_3:

TABLE 6
Round 4 of LAH Engineering

| | Fold Improvement | Fold Improvement |
|---|---|---|
| | total δ- | in % (R)-δ- |
| Mutant | dodecalactone | dodecalactone |
| G78P | 1.67 | 0.96 |
| L79V | 1.49 | 0.79 |
| E27A/C32A/Q45A/L154F | 1.47 | 1.03 |
| A363G | 1.46 | 0.95 |
| A363H | 1.46 | 0.73 |
| A335M | 1.45 | 0.80 |
| E27A/C32A/L154F | 1.45 | 1.06 |
| C32S | 1.45 | 1.07 |
| N359T | 1.42 | 0.63 |
| L79I | 1.39 | 0.79 |
| C32T | 1.39 | 0.99 |
| L445I | 1.38 | 1.21 |
| A363Y | 1.34 | 0.79 |
| L30I | 1.32 | 0.83 |
| A363F | 1.30 | 0.89 |
| L30N | 1.29 | 0.91 |
| T444S/L445I | 1.28 | 1.05 |
| C32D | 1.24 | 1.05 |
| A363W | 1.23 | 0.94 |
| C32A/Q45A/L154F | 1.23 | 1.04 |
| Q45A | 1.22 | 0.88 |
| C32E | 1.20 | 1.02 |
| T444A/L445I | 1.19 | 0.89 |
| A335G | 1.18 | 0.94 |
| L154F | 1.17 | 1.06 |
| L30A | 1.17 | 0.99 |
| A335L | 1.17 | 0.59 |
| T444Q | 1.16 | 1.07 |
| I441V | 1.13 | 0.89 |
| E27G | 1.13 | 0.96 |
| L30V | 1.13 | 0.90 |
| A335V | 1.13 | 0.77 |
| T337S | 1.11 | 0.97 |
| E27Q | 1.10 | 0.96 |
| V179T | 1.09 | 1.00 |
| Q53Q/P363A | 1.08 | 0.88 |
| I441L | 1.08 | 0.83 |
| T444S | 1.06 | 0.93 |
| E27D | 1.05 | 0.96 |
| A363S | 1.04 | 0.85 |
| H72N | 1.04 | 1.07 |
| S87N | 1.04 | 0.94 |
| N359H | 1.03 | 0.83 |
| E27P | 1.03 | 0.75 |
| Q45S | 1.00 | 1.09 |
| I441M | 0.98 | 1.03 |
| S87A | 0.98 | 1.05 |
| A363T | 0.97 | 1.01 |
| E27A | 0.97 | 1.01 |
| G99T | 0.92 | 1.06 |
| S87Q | 0.89 | 1.03 |
| A363M | 0.86 | 1.07 |
| S87K | 0.86 | 1.06 |
| G99S | 0.85 | 1.07 |
| S87R | 0.84 | 1.01 |
| S87H | 0.84 | 1.08 |
| T444N | 0.84 | 1.14 |
| D67N | 0.83 | 1.02 |
| L445V | 0.82 | 1.01 |
| H72I | 0.81 | 1.03 |
| G77N | 0.80 | 1.06 |
| S87G | 0.77 | 1.03 |
| G99N | 0.77 | 1.00 |
| T332S | 0.76 | 1.28 |
| A363L | 0.76 | 1.00 |
| M217L | 0.75 | 1.04 |
| T337L | 0.73 | 1.06 |
| T444S/L445V | 0.71 | 1.06 |
| A363I | 0.71 | 1.00 |
| I447M | 0.68 | 1.00 |
| M217I | 0.67 | 1.01 |
| T332P | 0.67 | 1.20 |
| I447Q | 0.63 | 1.12 |
| I157L | 0.62 | 1.01 |
| H72Q | 0.59 | 1.00 |
| T444Y | 0.57 | 1.02 |
| N359Y | 0.56 | 1.15 |
| T332A | 0.55 | 1.16 |
| P334S | 0.55 | 1.03 |
| N359F | 0.55 | 1.10 |
| T332G | 0.51 | 1.22 |
| T444A/L445M | 0.50 | 1.04 |
| H72M | 0.40 | 1.04 |
| T444A/L445A | 0.31 | 1.19 |
| L79F | 0.02 | 1.01 |

Based on the above results, SEQ ID NO: 63 derivative having T332S substitution was chosen as the next lead laurate ω-7 hydroxylase enzyme, LAH_4 (SEQ ID NO: 64).

### Example 6: Engineering of Yarrowia lipolytica Strain for Improved Production of Free Fatty Acids (FFA)

The yield of a lactone such as δ-dodecalactone may be increased by certain genetic modifications that improve the production of free fatty acids (FFA) from the substrate C12 fatty acid methyl ester (FAME) for conversion of the FFA to δ-dodecalactone. Therefore, Yarrowia lipolytica derivatives having deletions of genes encoding lipases, and/or genes involved in β-oxidation and peroxisome metabolism, fatty acid activation and degradation, and ω-oxidation were constructed. Specifically the following strains were constructed.
1. Wild type,
2. MFE1Δ (affecting β-oxidation and peroxisome metabolism),
3. LIP2Δ (deleting a lipase gene),
4. FAA1Δ, FAT1Δ (affecting fatty acid activation and degradation),
5 FAA1Δ, ALK5Δ (affecting (a) fatty acid activation and degradation, and (b) ω-oxidation),
6. FAA1Δ, FAT1Δ, ANT1Δ (affecting (a) fatty acid activation and degradation, and (b) β-oxidation and peroxisome metabolism),
7. FAA1Δ, ALK5Δ, ANT1Δ (affecting (a) fatty acid activation and degradation, (b) ω-oxidation, and (c) β-oxidation and peroxisome metabolism),
8. FAA1Δ, ALK5Δ, MFE1Δ (affecting (a) fatty acid activation and degradation, (b) ω-oxidation, and (c) β-oxidation and peroxisome metabolism)
9. FAA1Δ, FAT1Δ, LIP2Δ (affecting (a) fatty acid activation and degradation and (b) a lipase activity),
10. FAA1Δ, ALK5Δ, POX6Δ, POX3Δ (affecting (a) fatty acid activation and degradation, (b) ω-oxidation, and (c) β-oxidation and peroxisome metabolism)
11. FAA1Δ, ALK5Δ, POX6Δ, POX3Δ, POX5Δ, POX4Δ (affecting (a) fatty acid activation and degradation, (b) ω-oxidation, and (c) β-oxidation and peroxisome metabolism),
12. FAA1Δ, ALK5Δ, POX6Δ, POX3Δ, POX5Δ, POX4Δ, POX1Δ (affecting (a) fatty acid activation and degradation, (b) ω-oxidation, and (c) β-oxidation and peroxisome metabolism),
13. FAA1Δ, ALK5Δ, POX6Δ, POX3Δ, POX5Δ, POX4Δ, POX1Δ, POX2Δ (affecting (a) fatty acid activation and degradation, (b) ω-oxidation, and (c) β-oxidation and peroxisome metabolism),
14. FAA1Δ, FAT1Δ, LIP2Δ, POX3Δ (affecting (a) fatty acid activation and degradation, (b) a lipase activity, and (c) β-oxidation and peroxisome metabolism), and
15. FAA1Δ, FAT1Δ, LIP2Δ, POX3Δ, POX2Δ (affecting (a) fatty acid activation and degradation, (b) a lipase activity, and (c) β-oxidation and peroxisome metabolism).

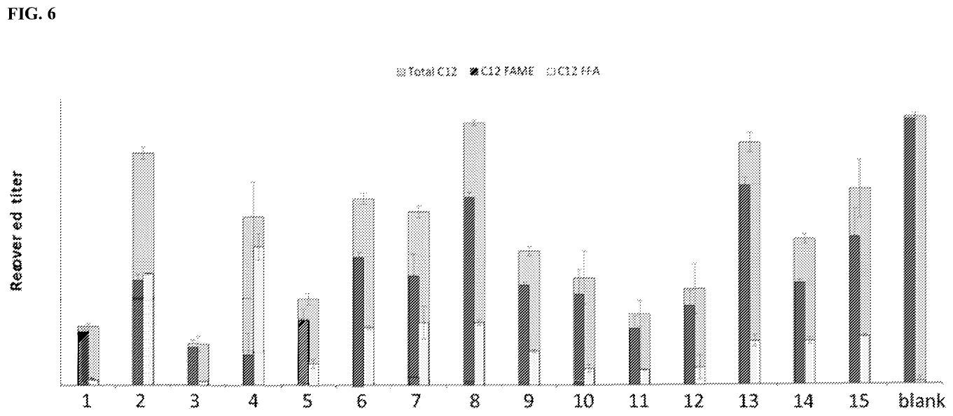

The above derivatives of Yarrowia lipolytica were analyzed for the production of free fatty acids. The strains pre-cultured for 24 hours in medium containing 20 g/L glucose at 30° C. and 900 rpm in a 96 deep well plate. The fermentations were carried out in a medium supplemented with 20 g/L C12 FAME for 48 hours in a 96 well plate. Blank medium was used as a control. After 48 hours the amount of FFA in each culture was analyzed. As shown in FIG. 6, the various mutant backgrounds improved the production of FAA. The medium alone (blank) had predominantly C12 FAME, as expected (FIG. 6). While wild type Yarrowia lipolytica metabolized FAME, as indicated by the total level of C12 fatty acids (C12 FAME+C12 FFA), very little FAA was detectable, indicating catabolism of the FFA generated by pathways involving lipases, β-oxidation and peroxisome metabolism, fatty acid activation and degradation, and ω-oxidation (FIG. 6). Deletions of genes involved in these pathways produced various effects. For example, deletions of some genes affecting fatty acid activation and degradation and β-oxidation and/or peroxisome metabolism produced increased FAA (see, example trains 2 and 4 in FIG. 6).

What is claimed is:

1. A microbial cell for producing one or more lactones, the microbial cell expressing an enzyme having fatty acid hydroxylase activity, wherein the microbial cell is engineered relative to a parent strain to have one or more modifications selected from:
(a) a decrease in expression and/or activity of one or more enzymes involved in fatty acid activation and degradation,
(b) a decrease in expression and/or activity of one or more β-oxidation and/or peroxisome enzymes;
(c) a modification in expression and/or activity of one or more lipases;
(d) a decrease in expression and/or activity of ω-oxidation enzymes; and
(e) a decrease in expression and/or activity of one or more acyl-CoA synthetase enzymes;
(f) an increase in metabolic NADPH supply;
(g) a decrease in expression and/or activity of one or more neutral lipid biosynthesis enzymes; and/or
(h) a decrease in expression and/or activity of one or more of citric acid cytoplasmic exporter, and one or more NADPH dependent aldehyde reductases.

2. The microbial cell of claim 2, wherein the enzyme is a cytochrome P450 enzyme selected from CYP505, CYP116, CYP703, and CYP102, or a derivative thereof.

3. The microbial cell of claim 1, wherein the P450 enzyme has a ω-7 hydroxylase activity on C12 fatty acid substrate.

4. The microbial cell of claim 2, wherein the P450 enzyme comprises an amino acid sequence that is at least 50% identical to the amino acid sequence selected from SEQ ID NOs: 1, 3, 4, 5, 8, and 11.

5.-10. (canceled)

11. The microbial cell of claim 2, wherein the P450 enzyme comprises an amino acid sequence that is at least 80%, or at least 85%, at least 90%, or at least 95%, or at least 97%, or at least 98%, or at least 99% identical to the amino acid sequence of SEQ ID NO: 29, and comprising one or more mutations or one or more sets of mutations that increase fatty acid hydroxylase activity or stereoselectivity as compared to the enzyme of SEQ ID NO: 29, wherein the one or more mutations or sets of mutations are selected from Tables 3, 4, 5, and 6.

12.-19. (canceled)

20. The microbial cell of claim 11, wherein the P450 enzyme comprises a substitution at one or more positions selected from Q53, T332, P363 and E443 with respect to SEQ ID NO: 29, wherein the substitutions are optionally selected from:
(i) Q53 is substituted with an amino acid selected from Arg, Ile, Leu, His, Lys, Gly, Ala, and Val;
(ii) T332 is substituted with Ser, Gly, Ala, Pro, Cys, Ile, Leu, and Val;
(iii) P363 is substituted with an amino acid selected from Ala, Leu, Ile, and Val; and
(iv) E443 is substituted with Gln or Asn.

21-22. (canceled)

23. The microbial cell of claim 1, wherein the modifications limit fatty acid activation and degradation.

24-26. (canceled)

27. The microbial cell of claim 1, wherein the modifications downregulate β-oxidation and peroxisome metabolism.

28-30. (canceled)

31. The microbial cell of claim 1, wherein the modifications comprise decreased expression or activity of lipases encoded by LIP2, LIP7, and/or LIP8 genes.

32-33. (canceled)

34. The microbial cell of claim 1, wherein the modifications comprise increased expression or activity of lipases encoded by LIP2, LIP7, and/or LIP8 genes.

35. The microbial cell of claim 1, wherein the microbial cell is engineered to downregulate ω-oxidation.

36-50. (canceled)

51. The microbial cell of claim 1, wherein the microbial cell is a yeast or fungal cell.

52-54. (canceled)

55. The microbial cell of claim 1, wherein the microbial cell is a bacterial cell belonging to a genus selected from Acidovorax, Acinetobacter, Actinomyces, Alcanivorax, Arthrobacter, Brevibacterium, Bacillus, Clostridium, Corynebacterium, Dietzia, Escherichia, Gordonia, Marinobacter, Mycobacterium, Micrococcus, Micromonospora, Moraxella, Nocardia, Pseudomonas, Psychrobacter, Rhodococcus, Salmonella, Streptomyces, Thalassolituus, and Thermomonospora.

56. (canceled)

57. A microbial cell for producing one or more lactones, the microbial cell expressing a P450 enzyme having fatty acid hydroxylase activity,
wherein the P450 enzyme comprises an amino acid sequence that has at least 80%, or at least 90%, or at least 95%, or at least 97%, or at least 98%, or 100% sequence identity to amino acids 1 to 461 of SEQ ID NO: 1, and comprises a CPR domain having at least 70% sequence identity, or at least 80% sequence identity, or at least 90% sequence identity, or at least 95% sequence identity, or at least 97% sequence identity, or at least 98% sequence identity or 100% sequence identity to a CPR domain selected from SEQ ID NOS: 37 to 60.

58-61. (canceled)

62. A method for making one or more lactones, comprising: culturing a microbial cell of claim 1 in the presence of a fatty acid substrate or an ester or glyceride thereof, and recovering the one or more lactones from the culture.

63-67. (canceled)

68. A method for making a product, comprising, incorporating one or more lactones made according to the method of claim 62 into said product.

69. (canceled)

70. A P450 enzyme having fatty acid hydroxylase activity, the P450 enzyme comprising an amino acid sequence that has at least 80% sequence identity to amino acids 1 to 461 of SEQ ID NO: 1, and comprises a CPR domain having at least 70% sequence identity to a CPR domain selected from SEQ ID NOS: 37 to 60.

71-76. (canceled)

77. A P450 enzyme having fatty acid hydroxylase activity, the P450 enzyme comprising an amino acid sequence that has at least 80% sequence identity to amino acids 1 to 461 of SEQ ID NO: 1, and comprises a CPR domain having at least 70% sequence identity to a CPR domain selected from SEQ ID NOs: 61 to 64, wherein the P450 enzyme comprises a substitution at one or more positions selected from Q53, T332, P363 and E443 with respect to SEQ ID NO: 29, wherein the substitutions are optionally selected from:
(i) Q53 is substituted with an amino acid selected from Arg, Ile, Leu, His, Lys, Gly, Ala, and Val;
(ii) T332 is substituted with Ser, Gly, Ala, Pro, Cys, Ile, Leu, and Val;
(iii) P363 is substituted with an amino acid selected from Ala, Leu, Ile, and Val; and
(iv) E443 is substituted with Gln or Asn.

78-79. (canceled)

80. A polynucleotide encoding the enzyme of claim 70.

81. (canceled)

82. A host cell expressing the polynucleotide of claim 80.

83. (canceled)
